SEQUENCE LISTING (16 sequences)

SEQ ID NO: 1 - DNA, 588 bp, Encephalomyocarditis virus
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 2 - DNA, 4528 bp, Artificial Sequence
  Mouse MAB2, promoter-LC-IRES-HC-WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggagacagatacactgctgctgtgggtgctgctcctctgggtgccaggatctacaggcgacatcgtgatgacccagagccacaagttcatgagcaccagcgtgggcgacagagtgtccatcacatgcaaggccagccaggacgtgaacacagccgtggcttggtatcagcagaagcccggccattctcctaagctgctgatctacagcgccagcttcagatacaccggcgtgcccgatagattcaccggcaacagaagcggcaccgacttcaccttcaccatcagctctgtgcaggccgaggatctggccgtgtactactgtcagcagcactacaccacacctccaaccttcggcggaggcaccaaggtggaaatcaagagagctgacgccgctcctaccgtgtctatcttcccacctagcagcgagcagctgacatctggcggagcctctgtcgtgtgcttcctgaacaacttctaccccaaggacatcaacgtgaagtggaagatcgacggcagcgagagacagaacggcgtgctgaactcttggaccgaccaggacagcaaggactccacctacagcatgagcagcaccctgacactgaccaaggacgagtacgagagacacaacagctacacatgcgaggctacccacaagaccagcacaagccccatcgtgaagtccttcaacagaaacgagtgctgaacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggagacagatacactgctgctgtgggtgctgctcctctgggtgccaggatctacaggccaggttcagctgcagcagtctggacctgagctggttaagcctggcgcctctctgaagctgagctgtaccgcttccggcttcaacatcaaggacacctacatccactgggtcaagcagaggcctgagcagggactcgagtggatcggcagaatctaccccaccaacggctacaccagatacgaccccaagttccaggacaaggccaccatcacagccgacaccagcagcaacacagcctatctccaggtgtccaggctgaccagcgaggacacagccgtgtactactgctctagatggggaggcgacggcttctacgccatggattattggggacagggcgccagcgtgacagtgtctagtgccaagacaacagcccctagcgtgtaccctctggctcctgtgtgtggcgacacaacaggcagctctgtgacactgggctgtctggtcaagggctacttccccgaaccagtgacactgacctggaacagcggctctctgtctagcggcgtgcacacatttccagccgtgctgcagagcgacctgtacacactgtcctctagcgtgaccgtgaccagctctacatggcccagccagagcatcacctgtaacgtggcccatcctgccagcagcaccaaggtggacaagaagatcgagcctagaggccctaccatcaagccctgtcctccatgcaagtgccccgctcctaatctgctcggaggcccaagcgtgttcatcttcccacctaagatcaaggacgtgctgatgatctctctgagccccatcgtgacctgcgtggtggtggatgtgtctgaggacgaccctgacgtgcagatcagttggttcgtgaacaacgtggaagtgcacacagcccagacacagacccacagagaggactacaacagcaccctgagagtggtgtctgccctgcctatccagcaccaggattggatgagcggcaaagaattcaagtgcaaagtgaacaacaaggacctgcctgctcctatcgagagaaccatcagcaagcccaagggctctgtcagggctcctcaggtgtacgttctgccacctcctgaggaagagatgaccaagaaacaagtgaccctcacctgtatggtcaccgacttcatgcccgaggacatctacgtggaatggaccaacaacggcaagaccgagctgaactacaagaacaccgagcctgtgctggacagcgacggcagctacttcatgtacagcaagctgcgcgtcgagaagaagaactgggtcgagagaaacagctacagctgctccgtggtgcacgagggactgcacaaccaccacaccaccaagagcttcagcagaacccctggcaagtgacaactttattatacatagttggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 3 - DNA, 4153 bp, Artificial Sequence
  mouse MAB2 - 2 promoters IgG, consisting of promoter 1 (CMV)-LC-polyA-promoter 2 (CMV)-HC-WPRE-poly A
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggagacagatacactgctgctgtgggtgctgctcctctgggtgccaggatctacaggcgacatcgtgatgacccagagccacaagttcatgagcaccagcgtgggcgacagagtgtccatcacatgcaaggccagccaggacgtgaacacagccgtggcttggtatcagcagaagcccggccattctcctaagctgctgatctacagcgccagcttcagatacaccggcgtgcccgatagattcaccggcaacagaagcggcaccgacttcaccttcaccatcagctctgtgcaggccgaggatctggccgtgtactactgtcagcagcactacaccacacctccaaccttcggcggaggcaccaaggtggaaatcaagagagctgacgccgctcctaccgtgtctatcttcccacctagcagcgagcagctgacatctggcggagcctctgtcgtgtgcttcctgaacaacttctaccccaaggacatcaacgtgaagtggaagatcgacggcagcgagagacagaacggcgtgctgaactcttggaccgaccaggacagcaaggactccacctacagcatgagcagcaccctgacactgaccaaggacgagtacgagagacacaacagctacacatgcgaggctacccacaagaccagcacaagccccatcgtgaagtccttcaacagaaacgagtgctgacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcacccagctttcttgtacaaagtgggccaccatggagacagatacactgctgctgtgggtgctgctcctctgggtgccaggatctacaggccaggttcagctgcagcagtctggacctgagctggttaagcctggcgcctctctgaagctgagctgtaccgcttccggcttcaacatcaaggacacctacatccactgggtcaagcagaggcctgagcagggactcgagtggatcggcagaatctaccccaccaacggctacaccagatacgaccccaagttccaggacaaggccaccatcacagccgacaccagcagcaacacagcctatctccaggtgtccaggctgaccagcgaggacacagccgtgtactactgctctagatggggaggcgacggcttctacgccatggattattggggacagggcgccagcgtgacagtgtctagtgccaagacaacagcccctagcgtgtaccctctggctcctgtgtgtggcgacacaacaggcagctctgtgacactgggctgtctggtcaagggctacttccccgaaccagtgacactgacctggaacagcggctctctgtctagcggcgtgcacacatttccagccgtgctgcagagcgacctgtacacactgtcctctagcgtgaccgtgaccagctctacatggcccagccagagcatcacctgtaacgtggcccatcctgccagcagcaccaaggtggacaagaagatcgagcctagaggccctaccatcaagccctgtcctccatgcaagtgccccgctcctaatctgctcggaggcccaagcgtgttcatcttcccacctaagatcaaggacgtgctgatgatctctctgagccccatcgtgacctgcgtggtggtggatgtgtctgaggacgaccctgacgtgcagatcagttggttcgtgaacaacgtggaagtgcacacagcccagacacagacccacagagaggactacaacagcaccctgagagtggtgtctgccctgcctatccagcaccaggattggatgagcggcaaagaattcaagtgcaaagtgaacaacaaggacctgcctgctcctatcgagagaaccatcagcaagcccaagggctctgtcagggctcctcaggtgtacgttctgccacctcctgaggaagagatgaccaagaaacaagtgaccctcacctgtatggtcaccgacttcatgcccgaggacatctacgtggaatggaccaacaacggcaagaccgagctgaactacaagaacaccgagcctgtgctggacagcgacggcagctacttcatgtacagcaagctgcgcgtcgagaagaagaactgggtcgagagaaacagctacagctgctccgtggtgcacgagggactgcacaaccaccacaccaccaagagcttcagcagaacccctggcaagtgacaactttattatacatagttggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 4 - DNA, 4483 bp, Artificial Sequence
  Mouse MAB1, promoter-LC-IRES shorter-HC-WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggagacagatacactgctgctgtgggtgctgctcctctgggtgccaggatctacaggcgacgtggtcatgacacagacccctctgacactgagcgtgaccatcggacagcctgccagcatcagctgcaagagcagtcagagcctgctgcacagcgacggcaagacctacctgaactggctgctgcaaagacccggccagtctcctaagaggctgatctacctggtgtccaagctggacagcagaatccccgacagattcacaggcagcggctctggcacagacttcaccctgaagatcagcagagtggaagccgaggacctgggcgtgtactactgttggcagggcacacacttccctcacacattcggcgctggcacaaagctggaactgaagagagctgacgccgctcctaccgtgtctatcttcccacctagcagcgagcagctgacatctggcggagcctctgtcgtgtgcttcctgaacaacttctaccccaaggacatcaacgtgaagtggaagatcgacggcagcgagagacagaacggcgtgctgaactcttggaccgaccaggacagcaaggactccacctacagcatgagcagcaccctgacactgaccaaggacgagtacgagagacacaacagctacacatgcgaggctacccacaagaccagcacaagccccatcgtgaagtccttcaacagaaacgagtgctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggagacagatacactgctgctgtgggtgctgctcctctgggtgccaggatctacaggcgaggttcagctgcagcagtctggacctgagctggttaagcctggcgcctccgtgaagatcagctgcaagacaagcggcttcaccttcaccgagtacagcatgcactgggtcaagcagagccacggcaagagcctggaatggatcggcggcatcaaccctaacaacggcggcaccagctacaaccagaagttcaagggcaaagccacactgaccgtggacaagagcagcagcaccgcctacatggaactgagaagcctgaccagcgaggacagcgccgtgtactactgtgccagagagtcttggggccagggcacaaccctgacagtctcttctgccaagacaacagcccctagcgtgtaccctctggctcctgtgtgtggcgacacaacaggcagctctgtgacactgggctgtctggtcaagggctacttccccgaaccagtgacactgacctggaacagcggctctctgtctagcggcgtgcacacatttccagccgtgctgcagagcgacctgtacacactgtcctctagcgtgaccgtgaccagctctacatggcccagccagagcatcacctgtaacgtggcccatcctgccagcagcaccaaggtggacaagaagatcgagcctagaggccctaccatcaagccctgtcctccatgcaagtgccccgctcctaatctgctcggaggcccaagcgtgttcatcttcccacctaagatcaaggacgtgctgatgatctctctgagccccatcgtgacctgcgtggtggtggatgtgtctgaggacgaccctgacgtgcagatcagttggttcgtgaacaacgtggaagtgcacacagcccagacacagacccacagagaggactacaacagcaccctgagagtggtgtctgccctgcctatccagcaccaggattggatgagcggcaaagaattcaagtgcaaagtgaacaacaaggacctgcctgctcctatcgagagaaccatcagcaagcccaagggctctgtcagggctcctcaggtgtacgttctgccacctcctgaggaagagatgaccaagaaacaagtgaccctcacctgtatggtcaccgacttcatgcccgaggacatctacgtggaatggaccaacaacggcaagaccgagctgaactacaagaacaccgagcctgtgctggacagcgacggcagctacttcatgtacagcaagctgcgcgtcgagaagaagaactgggtcgagagaaacagctacagctgctccgtggtgcacgagggactgcacaaccaccacaccaccaagagcttcagcagaacccctggcaagtgaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 5 - DNA, 4516 bp, Artificial Sequence
  MAB1 promoter-LC-IRES-HC-WPRE-PolyA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggagacagatacactgctgctgtgggtgctgctcctctgggtgccaggatctacaggcgacgtggtcatgacacagacccctctgacactgtccgtgaccatcggacagcctgcctccatctcctgcaagtcctctcagtccctgctgcactctgacggcaagacctacctgaactggctgctgcagaggcctggccagagtcctaagagactgatctacctggtgtccaagctggactctcggatccctgacagattcaccggctctggctctggcaccgacttcaccctgaagatctccagagtggaagccgaggacctgggcgtgtactactgttggcagggcacccactttccacacacctttggcgctggcacaaagctggaactgaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgttgaacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggagacagatacactgctgctgtgggtgctgctcctctgggtgccaggatctacaggcgaggttcagctgcagcagtctggacctgagctggttaagcctggcgcctccgtgaagatctcctgcaagacctctggcttcaccttcaccgagtactccatgcactgggtcaagcagtcccacggcaagtccctggaatggatcggcggcatcaaccctaacaacggcggcacctcctacaaccagaagttcaagggcaaagctaccctgaccgtggacaagtcctcctccaccgcctacatggaactgcggtccctgacctctgaggactccgccgtgtactactgcgctagagagtcttggggccagggcaccacactgacagtctcttctgcttctaccaagggacccagcgtgttccctctggctccttccagcaagtctacctctggcggaacagctgctctgggctgcctggtcaaggactactttcctgagcctgtgaccgtgtcttggaactctggcgctctgacatccggcgtgcacacctttccagctgtgctgcaatccagcggcctgtactctctgtcctccgtcgtgaccgtgccttctagctctctgggcacacagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggaagagatgaccaagaaccaggtgtccctgacctgtctcgtgaagggcttctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgagccccggcaagtagcaactttattatacatagttggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 6 - DNA, 4141 bp, Artificial Sequence
  MAB1 - 2 promoters IgG, consisting of promoter 1 (CMV)-LC-polyA-promoter 2 (CMV)-HC-WPRE-poly A
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggagacagatacactgctgctgtgggtgctgctcctctgggtgccaggatctacaggcgacgtggtcatgacacagacccctctgacactgtccgtgaccatcggacagcctgcctccatctcctgcaagtcctctcagtccctgctgcactctgacggcaagacctacctgaactggctgctgcagaggcctggccagagtcctaagagactgatctacctggtgtccaagctggactctcggatccctgacagattcaccggctctggctctggcaccgacttcaccctgaagatctccagagtggaagccgaggacctgggcgtgtactactgttggcagggcacccactttccacacacctttggcgctggcacaaagctggaactgaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgttgacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcacccagctttcttgtacaaagtgggccaccatggagacagatacactgctgctgtgggtgctgctcctctgggtgccaggatctacaggcgaggttcagctgcagcagtctggacctgagctggttaagcctggcgcctccgtgaagatctcctgcaagacctctggcttcaccttcaccgagtactccatgcactgggtcaagcagtcccacggcaagtccctggaatggatcggcggcatcaaccctaacaacggcggcacctcctacaaccagaagttcaagggcaaagctaccctgaccgtggacaagtcctcctccaccgcctacatggaactgcggtccctgacctctgaggactccgccgtgtactactgcgctagagagtcttggggccagggcaccacactgacagtctcttctgcttctaccaagggacccagcgtgttccctctggctccttccagcaagtctacctctggcggaacagctgctctgggctgcctggtcaaggactactttcctgagcctgtgaccgtgtcttggaactctggcgctctgacatccggcgtgcacacctttccagctgtgctgcaatccagcggcctgtactctctgtcctccgtcgtgaccgtgccttctagctctctgggcacacagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggaagagatgaccaagaaccaggtgtccctgacctgtctcgtgaagggcttctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgagccccggcaagtagcaactttattatacatagttggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 7 - DNA, 3274 bp, Artificial Sequence
  MAB1, promoter-scFv-Fc-WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggagacagatacactgctgctgtgggtgctgctcctctgggtgccaggatctacaggcgaggttcagctgcagcagtctggacctgagctggttaagcctggcgcctccgtgaagatctcctgcaagacctctggcttcaccttcaccgagtactccatgcactgggtcaagcagtcccacggcaagtccctggaatggatcggcggcatcaaccctaacaacggcggcacctcctacaaccagaagttcaagggcaaagctaccctgaccgtggacaagtcctcctccaccgcctacatggaactgcggtccctgacctctgaggactccgccgtgtactactgcgctagagagtcttggggccagggcaccacactgacagtctcttctggaggcggaggatctggcggaggtggaagtggcggaggcggatctgacgtggtcatgacacagacccctctgacactgtccgtgaccatcggacagcctgcctccatctcctgcaagtcctctcagtccctgctgcactctgacggcaagacctacctgaactggctgctgcagaggcctggccagagtcctaagagactgatctacctggtgtccaagctggactctcggatccctgacagattcaccggctctggctctggcaccgacttcaccctgaagatctccagagtggaagccgaggacctgggcgtgtactactgttggcagggcacccactttccacacacctttggcgctggcacaaagctggaactgaagggaggcggaggatctgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggaagagatgaccaagaaccaggtgtccctgacctgtctcgtgaagggcttctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgagccccggcaagtagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 8 - DNA, 576 bp, Encephalomyocarditis virus
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataat

SEQ ID NO: 9 - DNA, 3859 bp, Artificial Sequence
  MAB1 FAB promoter-LC-IRES-HC-WPRE-PolyA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggagacagatacactgctgctgtgggtgctgctcctctgggtgccaggatctacaggcgacgtggtcatgacacagacccctctgacactgtccgtgaccatcggacagcctgcctccatctcctgcaagtcctctcagtccctgctgcactctgacggcaagacctacctgaactggctgctgcagaggcctggccagagtcctaagagactgatctacctggtgtccaagctggactctcggatccctgacagattcaccggctctggctctggcaccgacttcaccctgaagatctccagagtggaagccgaggacctgggcgtgtactactgttggcagggcacccactttccacacacctttggcgctggcacaaagctggaactgaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgttgaacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggagacagatacactgctgctgtgggtgctgctcctctgggtgccaggatctacaggcgaggttcagctgcagcagtctggacctgagctggttaagcctggcgcctccgtgaagatctcctgcaagacctctggcttcaccttcaccgagtactccatgcactgggtcaagcagtcccacggcaagtccctggaatggatcggcggcatcaaccctaacaacggcggcacctcctacaaccagaagttcaagggcaaagctaccctgaccgtggacaagtcctcctccaccgcctacatggaactgcggtccctgacctctgaggactccgccgtgtactactgcgctagagagtcttggggccagggcaccacactgacagtctcttctgcttctaccaagggacccagcgtgttccctctggctccttccagcaagtctacctctggcggaacagctgctctgggctgcctggtcaaggactactttcctgagcctgtgaccgtgtcttggaactctggcgctctgacatccggcgtgcacacctttccagctgtgctgcaatccagcggcctgtactctctgtcctccgtcgtgaccgtgccttctagctctctgggcacacagacctacatctgcaatgtgaaccacaagccttccaacaccaaggtggacaagaaggtggaacccaagtcctgcggctcccaccaccatcaccatcattagcaactttattatacatagttggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 10 - DNA, 4088 bp, Artificial Sequence
  MAB1 Fab - 2 promoters IgG, consisting of promoter 1 (CMV)-HC-polyA-promoter 2 (CMV)-LC-WPRE-poly A
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggagacagatacactgctgctgtgggtgctgctcctctgggtgccaggatctacaggcgaggttcagctgcagcagtctggacctgagctggttaagcctggcgcctccgtgaagatctcctgcaagacctctggcttcaccttcaccgagtactccatgcactgggtcaagcagtcccacggcaagtccctggaatggatcggcggcatcaaccctaacaacggcggcacctcctacaaccagaagttcaagggcaaagctaccctgaccgtggacaagtcctcctccaccgcctacatggaactgcggtccctgacctctgaggactccgccgtgtactactgcgctagagagtcttggggccagggcaccacactgacagtctcttctgcttctaccaagggacccagcgtgttccctctggctccttccagcaagtctacctctggcggaacagctgctctgggctgcctggtcaaggactactttcctgagcctgtgaccgtgtcttggaactctggcgctctgacatccggcgtgcacacctttccagctgtgctgcaatccagcggcctgtactctctgtcctccgtcgtgaccgtgccttctagctctctgggcacacagacctacatctgcaatgtgaaccacaagccttccaacaccaaggtggacaagaaggtggaacccaagtcctgcggctcccaccaccatcaccatcattagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcacccagctttcttgtacaaagtgggccaccatggagacagatacactgctgctgtgggtgctgctcctctgggtgccaggatctacaggcgacgtggtcatgacacagacccctctgacactgtccgtgaccatcggacagcctgcctccatctcctgcaagtcctctcagtccctgctgcactctgacggcaagacctacctgaactggctgctgcagaggcctggccagagtcctaagagactgatctacctggtgtccaagctggactctcggatccctgacagattcaccggctctggctctggcaccgacttcaccctgaagatctccagagtggaagccgaggacctgggcgtgtactactgttggcagggcacccactttccacacacctttggcgctggcacaaagctggaactgaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgttgacaactttattatacatagttggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 11 - protein, 24 aa, Artificial Sequence
  Expected LC C-terminal sequence
VYACEVTHQGLSSPVTKSFNRGEC

SEQ ID NO: 12 - protein, 64 aa, Artificial Sequence
  Analyzed LC C-terminal sequence
VYACEVTHQGLSSPVTKSFNRGECRKRRSGSGAPVKQTLNFDLLKLAGDVESNPGPMETDTLLL

SEQ ID NO: 13 - protein, 20 aa, Artificial Sequence
  Expected HC C-terminal sequence
MHEALHNHYTQKSLSLSPGK

SEQ ID NO: 14 - protein, 59 aa, Artificial Sequence
  Analyzed HC C-terminal sequence
MHEALHNHYTQKSLSLSPGKRKRRSGSGAPVKQTLNFDLLKLAGDVESNPGPMETDTLL

SEQ ID NO: 15 - protein, 7 aa, Artificial Sequence
  BBB targeting peptide
NRGTEWD

SEQ ID NO: 16 - protein, 8 aa, Artificial Sequence
  core sequence motif of self-cleavage peptides is the 2A peptide family
  misc_feature (2)..(2): Xaa can be any naturally occurring amino acid
  misc_feature (4)..(4): Xaa can be any naturally occurring amino acid
DXEXNPGP

(19) United States
(12) Patent Application Publication
(10) Pub. No.: US 2024/0100187 A1
(43) Pub. Date: Mar. 28, 2024

(54) Antibody Delivery

(71) Applicant: AC Immune SA, Lausanne (CH)

(72) Inventors: Gregorio Del Val, Lausanne (CH); Damien Névoltris, Lausanne (CH)

(21) Appl. No.: 18/039,840

(22) PCT Filed: Dec. 17, 2021
(86) PCT No.: PCT/EP2021/086645

§ 371 Date: Jun. 1, 2023

(30) Foreign Application Priority Data
Dec. 18, 2020 (EP) 20215823.4

(51) Int. Cl. A61K 48/00; C07K 16/18; C12N 15/86

(52) U.S. Cl. CPC A61K 48/0058; A61K 48/0066; C07K 16/18; C12N 15/86; C07K 2317/622; C12N 2750/14143

(57) ABSTRACT

A vector comprises a polynucleotide encoding an antibody or antibody fragment for use in a method of treatment of a disease or disorder of the central nervous system (CNS) in a subject, wherein the vector transduces or transfects cells of the blood brain barrier (BBB) and the transduced or transfected BBB cells express the antibody or antibody fragment resulting in delivery of the antibody or antibody fragment into the CNS, preferably into the brain parenchyma. Expression cassettes useful in such vectors may comprise from 5′ to 3′: at least one promoter operably linked to a first gene encoding a light chain of an antibody or antibody fragment and to a second gene encoding a heavy chain of the antibody or antibody fragment and further comprise an IRES after the first gene encoding the light chain of the antibody or antibody fragment and before the second gene encoding the heavy chain of the antibody or antibody fragment or a first promoter operably linked to a first gene encoding a light chain of an antibody or antibody fragment and a second promoter operably linked to a second gene encoding a heavy chain of the antibody or antibody fragment. The antibodies and antibody fragments thus produced may be of higher quality, displaying lower levels of aggregation and unwanted immunogenicity.

### FIELD OF THE INVENTION

The present invention relates to means and methods for effective delivery of genes coding for an antibody or antibody fragment via a vector, such as a viral vector amongst others, to cells from the blood-brain barrier (BBB) or the central nervous system (CNS), such as brain endothelial cells, to produce an antibody molecule(s) in the cells that is released to the CNS, preferably into the brain parenchyma. By delivering antibody genes into the BBB or CNS, in particular brain endothelial cells, the invention also relates to a method of increasing antibody concentration in the central nervous system (CNS). Delivery of a therapeutic antibody by this approach is useful to treat various diseases or disorders that originate in the CNS, such as neurodegenerative diseases or disorders, movement diseases or disorders, brain-related tumors, psychosis and CNS neuroinflammation amongst others. In one aspect, the invention stems from the unexpected discovery that transduction of brain endothelial cells in vitro by vectors such as adeno-associated virus (AAV) vectors leads to secretion of high quality antibodies in large quantity into the basolateral space. The invention also describes a surprisingly high antibody expression yield, and an improved protein quality by alternating the chain positions, using different secretion peptides, and customizing up-stream, intra- and down-stream regulatory elements. The present invention bypasses the difficulties associated with delivery of therapeutic antibodies to the brain through the need to cross the blood brain barrier in sufficient therapeutic closes. The present invention is applicable to any mammal, especially human subjects and aims to improve the delivery of therapeutic antibodies to the brain. The present invention may thus be employed for the treatment of diseases, disorders or conditions associated of a patient who is suffering from a CNS disease or disorder, including but not limited to diseases associated with amyloid-beta protein, TDP-43-proteinopathies, alpha-synucleinopathies, Tauopathies, trinucleotide repeat disorders including poly-glutamine disorders such as Huntington's disease, brain-related cancers and tumors, epilepsy, psychiatric diseases, neuroinflammatory diseases, neuromuscular diseases, viral-induced encephalitis and diseases characterized by microglial dysfunction.

### BACKGROUND OF THE INVENTION

The blood brain barrier (BBB) is a structural and functional barrier which protects the brain from blood borne pathogens and toxins but also maintains a tightly regulated microenvironment required for proper neuronal functioning into central nervous system (CNS) [1, 2]. The BBB is composed of three cells types: the endothelial cells (ECs) which form the physical barrier between the bloodstream and the brain, and two mural cells, pericytes and astrocytes, that sit on the abluminal surface of the EC layer [3]. Although brain endothelial cells contribute to the main functions of the BBB, these three cell types compose the entire blood-brain barrier of most vertebrates. Their interactions and communication is critical to maintain a tightly regulated CNS homeostasis.

Brain endothelial cells have unique properties compared with endothelial cells; they are held together by tight junctions (TJ), a multiprotein junctional network that limits the diffusion of compound thought the paracellular space. While small molecules like water, oxygen and small lipid soluble substances can easily cross from the blood into the brain, the tight junctions prevent diffusion of large molecules entering the brain, including antibodies. [4]

This tightly regulated entry to the brain is one of the main challenges for development of biologics, for diseases involving the central nervous system (CNS). Indeed, it has been reported that around 0.1%-0.3% of the injected antibody reaches the brain following peripheral administration [5] [6]. Even with a high injected close, it could be often challenging to achieve sufficient concentration of antibody in the brain to elicit a therapeutic response. This drawback has been described to be one of the possible causes of failure of passive immunotherapies during clinical trials. ([7])

During the past decades, alternative non-invasive approaches like receptor mediated transcytosis ([8]), nanoparticle delivery [9] intranasal delivery [11, 12] or cell-based technologies ([13, 14]) have been developed to increase macromolecule penetration in the brain. However, limited efficacy were reported, mainly linked to manufacturing challenges[15], in vivo target mediated drug disposition [16] [17] or suspected antigenicity of modified protein [18].

In a recent study [14], authors reported a cell targeted delivery system consisting of ex vivo transfected, autologous endothelial precursor cells (EPCs) capable of homing to the BBB and expressing therapeutic antibody. However, even though successful ex vivo transfection was observed, limited homing of engineered EPC after intravenous transplantation is expected [19], reducing the therapeutic potential of such an approach. Intra-arterial injection could be a suitable alternative but remains challenging as cell close, infusion speed, and cell types must be carefully evaluated and optimized.

According to gene therapies using viral vectors, a gene of interest may be delivered directly into the CNS based on the introduction of therapeutic genes into a specific organ, tissue, or cell type using viruses which have been modified to include a transgene of interest in their genome. Viral vectors which have been used for gene delivery therapy are based, but not limited to retroviruses, lentiviruses, adenoviruses and adeno-associated viruses.

Adeno-associated viruses (AAVs) present an efficient, and clinically safe platform for gene delivery as evidenced by recent approvals: Luxturna® (voretigene neparvovec-rzyl), the first AAV2-based therapy approved delivers a functional copy of the RPE65 gene to patients with inherited retinal disease due to mutations in both copies of the RPE65 gene and Zolgensma® (onasemnogene abeparvovec-xioi), an AAV9 vector delivering a functional copy of the SMN1 gene to motor neurons in spinal muscular atrophy (SMA) patients.

AAVs have also been used as vehicles for gene transfer to the nervous system enabling gene expression, knockdown, and gene editing [21]. However, most of these applications rely on invasive, local injection of AAV Vectors, (intraventricular, intrathecal or intracisternal administration), in order to 1) bypass the blood-brain barrier and 2) temporally and spatially restrict transgene expression.

Different wild type serotypes, including AAV2 can transduce the BBB, but do not cross it, thus invasive surgeries are required for their delivery to the brain [22, 23]. In contrast, AAV9 serotypes administered intravenously can overcome the BBB and enter the CNS, resulting in gene transfer to the brain and spinal cord [24, 25].

Recently reported engineered capsid vectors, such as but not limited to AAV-S, AAV-F or AAV-PHP.eB based on AAV9 ([27-29]), provide good brain transduction, where a majority of neurons and astrocytes across many regions of the adult mouse were transduced using an intravenous route of administration. Similarly, the AAV-BR1 (referred to interchangeably herein s AAV2-BR1) capsid [30], based on AAV2, or more recently, AAV9-PHP.V1 were reported to selectively transduce brain endothelial cells with long lasting transgene expression, with the potential to treat neurovascular diseases. AAV-mediated expression of either whole immunoglobulins (IgG) or antibody fragments devoid of Fc domains were demonstrated within the CNS for various indications [32-44] but inherent limitations for both formats were also reported Indeed, the packaging size of AAV expression cassettes imposes design constraints for whole IgG genes where all required elements for transcription and translation including both heavy chain and light chain genes need to be under 4.7 kb. To date, most constructs consist of a single-promoter and therefore require the use of self-processing sequences such as Furin-2A (F2A) between the antibody heavy chain gene (HC) and the light chain (LC) [47-49]. Researchers have a significant preference for this construct due to high expression titers, small F2A sequence size (only 60 to 80 base pairs) and equimolarity in antibody chains expression. However, and in most cases, the F2A peptide remains attached to either the heavy or light chains, potentially triggering unwanted immunogenicity of the expressed proteins or antibody expressing cells [45]. Internal ribosomal entry site (IRES) for bicistronic antibody expression has been described as an alternative to self-cleaving sequences but lower protein expression is typically obtained due to an imbalance in heavy chain and light chain expression. Whilst antibody fragments such as scFv or single domain antibodies present some advantages such as higher protein titers as compared to whole IgG molecule due to monocistronic expression, said fragments lack Fc effector functions and do not have FcRn binding capacity leading to shorter half-life in vivo. Fragments devoid of Fc domain are then not able to recruit effector cells to clear pathological complexes in brain parenchyma and present reduced efflux of bound antigen from the brain to the blood via reverse transcytosis [32, 50-52]. Due to the monovalent binding capacity of such molecules, a lower affinity is observed as compared to the IgG counterpart. As such, secreted whole IgG to target and clear extracellular proteinopathies may be the preferred option as compared to fragments, but there is a need to develop a method that circumvents current limitations, allowing high quality expression to increase antibody exposure, recruiting effector cells and lowering risk of unwanted immunogenicity of in situ produced IgG.

### DESCRIPTION OF THE INVENTION

Here we describe for the first time a delivery method that increases (therapeutic) antibody or antibody fragment concentration in the central nervous system (CNS), by delivering genes coding for an antibody or antibody fragments using a vector, such as a viral vector, or another vector such as liposomes, or nanoparticles, to cells from the blood-brain barrier (BBB), such as but not limited to brain endothelial cells, to locally produce the therapeutic antibody molecule into the CNS, preferably into the brain parenchyma. Antibodies produced by this novel approach may be used to treat CNS-related disorders. In such a scenario, cells of the BBB (brain endothelial cells and/or pericytes and/or astrocytes) provide for long term expression of high quality antibodies into the CNS, in particular the brain parenchyma.

This novel strategy bypasses the hindrance of conventional passive immunization strategies that need to cross the blood brain barrier to reach sufficient therapeutic doses of antibody into the CNS, preferably into the brain parenchyma.

The invention also relates to improved expression cassettes for production of antibodies and antibody fragments that produce an unexpected higher antibody expression yield, and an improved protein quality by alternating the chain positions, using different secretion peptides, and customizing intra- and down-stream regulatory elements with respect to the previously reported strategies in the prior art.

The invention is based in part on the discovery that vectors can be used to deliver polynucleotide(s) encoding an antibody or antibody fragment to cells of the blood brain barrier (BBB). Expression of the polynucleotide(s) encoding an antibody or antibody fragment by the cells of the BBB leads to delivery of the antibody or antibody fragment into the CNS, preferably into the abrain parenchyma. This discovery presents a novel strategy that bypasses the limitations of conventional strategies that rely upon therapeutic antibodies crossing the BBB in order to reach sufficient therapeutic doses of antibody into the CNS, preferably into the brain parenchyma, to treat diseases or disorders of the CNS.

, in one aspect, the invention provides a vector comprising polynucleotide(s) encoding an antibody or antibody fragment for use in a method of treatment of a disease or disorder of the central nervous system (CNS) in a subject, wherein the vector transduces or transfects cells of the blood brain barrier (BBB), and the transduced or transfected BBB cells express the antibody or antibody fragment resulting in delivery of the antibody or antibody fragment into the CNS.

By targeting the vectors comprising a polynucleotide encoding an antibody or antibody fragment described herein to the BBB, there is an increase in the amount of antibodies or antibody fragments that are delivered to the CNS, preferably into the brain parenchyma, as compared to conventional antibody therapeutics that rely on the direct delivery of antibodies or antibody fragments to a subject. As described elsewhere, it has been reported that only around 0.1%-0.3% of the injected antibody reaches the brain following peripheral administration. In contrast, the inventors have discovered that using an AAV vector to introduce polynucleotide(s) encoding an antibody (MAB1) into a brain endothelial cell line results in unpolarized secretion of MAB1. This means that this technique may be employed for expression of antibodies or antibody fragments into the CNS, preferably into the brain parenchyma.

The CNS consists of two main components, the brain, and the spinal cord. Sensory impulses are transmitted to the CNS and motor impulses pass from the CNS. The CNS also coordinates the activity of the entire nervous system. In one embodiment, the vectors for use in accordance with the invention transduce or transfect cells of the blood brain barrier (BBB) and the transduced or transfected BBB cells express the antibody or antibody fragment resulting in delivery of the antibody or antibody fragment into the CNS. The CNS comprises multiple cell-types and in one embodiment the antibody or antibody fragment is delivered into at least one (up to all) cell-type in the CNS. In one embodiment, the antibody or antibody fragment may be delivered to: brain endothelial cells, neurons, pericytes, astrocytes, oligodendrocytes, microglia and ependymal cells. Thus, delivery may be to neuronal and non-neuronal (glial) cells of the CNS. In one embodiment, the antibody or antibody fragment is secreted from the BBB cells into the CNS. For example, at least 20% of the expressed antibody or antibody fragment in the BBB cells is delivered into the CNS. In one embodiment, at least 30% of the expressed antibody or antibody fragment in the BBB cells is delivered into the CNS. In one embodiment, at least 40% of the expressed antibody or antibody fragment in the BBB cells is delivered into the CNS. In one embodiment, at least 50% of the expressed antibody or antibody fragment in the BBB cells is delivered into the CNS. In one embodiment, at least 60% of the expressed antibody or antibody fragment in the BBB cells is delivered into the CNS. In one embodiment, at least 70% of the expressed antibody or antibody fragment in the BBB cells is delivered into the CNS. In one embodiment, at least 80% of the expressed antibody or antibody fragment in the BBB cells is delivered into the CNS. In one embodiment, at least 90% of the expressed antibody or antibody fragment in the BBB cells is delivered into the CNS.

In a preferred embodiment, the antibody or antibody fragment is delivered into the brain parenchyma. As used herein “brain parenchyma” refers to the functional tissue of the brain that is composed of neurons and glial cells. The vectors for use in accordance with the invention transduce or transfect cells of the blood brain barrier (BBB) and the transduced or transfected BBB cells express the antibody or antibody fragment resulting in delivery of the antibody or antibody fragment into the brain parenchyma. In one embodiment, the antibody or antibody fragment is secreted from the BBB cells into the brain parenchyma. For example, at least 20% of the expressed antibody or antibody fragment in the BBB cells is delivered into the brain parenchyma. In one embodiment, at least 30% of the expressed antibody or antibody fragment in the BBB cells is delivered into the brain parenchyma. In one embodiment, at least 40% of the expressed antibody or antibody fragment in the BBB cells is delivered into the brain parenchyma. In one embodiment, at least 50% of the expressed antibody or antibody fragment in the BBB cells is delivered into the brain parenchyma. In one embodiment, at least 60% of the expressed antibody or antibody fragment in the BBB cells is delivered into the brain parenchyma. In one embodiment, at least 70% of the expressed antibody or antibody fragment in the BBB cells is delivered into the brain parenchyma. In one embodiment, at least 80% of the expressed antibody or antibody fragment in the BBB cells is delivered into the brain parenchyma. In one embodiment, at least 90% of the expressed antibody or antibody fragment in the BBB cells is delivered into the brain parenchyma.

As already described elsewhere, the BBB is a structural and functional barrier that protects the brain from blood borne pathogens and toxins. The BBB is comprised of three cell-types: the endothelial cells, pericytes and astrocytes. Whilst the inventors' findings primarily relate to endothelial cells of the BBB, their observations may be equally applicable to other cells of the BBB. As such, in one embodiment the vector transduces or transfects the polynucleotides(s) encoding an antibody or antibody fragment into a cell-type selected from: the endothelial cells, pericytes and astrocytes. The vector may transduce or transfect one particular cell-type of the BBB. For example, the vector may transduce or transfect the pericytes of the BBB. In a further example, the vector may transduce or transfect the astrocytes of the BBB. As mentioned above, following transduction or transfection, the antibody or antibody fragment is expressed.

In a preferred embodiment, the vector transfects or transduces the endothelial cells of the BBB. Transfection or transduction of the BBB endothelial cells with the polynucleotide described herein is preferred because the BBB endothelial cells have a slow turn-over rate which may prove optimal for long-term expression in vivo of the antibody or antibody fragment into the CNS, preferably into the brain parenchyma.

Alternatively, the vector may transduce or transfect a plurality of cell-types of the BBB. For example, the vector may transduce or transfect the endothelial cells and the astrocytes of the BBB. Alternatively, the vector may transduce or transfect the endothelial cells and the pericytes of the BBB. As a further alternative, the vector may transduce or transfect the astrocytes and the pericytes of the BBB. In another alternative, the vector may transduce or transfect the endothelial cells, the astrocytes and the pericytes of the BBB. As mentioned above, following transduction or transfection, the antibody or antibody fragment is expressed.

In some embodiments, the vector comprising polynucleotide(s) encoding an antibody or antibody fragment may selectively target cells of the BBB. Targeting to particular cells of the BBB can be achieved, for example, by using neurotropic vectors. Neurotropic vectors may be viral vectors, which term includes engineered versions. Viral vectors are typically replication incompetent. The polynucleotide(s) may integrate into the genome of the cells of the BBB. One example of a neurotropic vector is herpes simplex virus (HSV). As used herein “neurotropic vector” refers to a vector that preferentially transduces or transfects cells of the BBB and/or CNS as compared to non-BBB and/or non-CNS cells respectively. Therefore, in one embodiment, the vector comprises a neurotropic vector. In another embodiment, the vector comprises a modified HSV.

Alternative routes to selectively target cells of the BBB is to utilize a vector expressing or comprising (particularly on the surface, such as in the viral capsid) a peptide, small molecule (SME), antibody or antibody fragment thereof, protein, nanoparticle, lipid, oligonucleotide, aptamer or cationic molecule on the vector surface that targets the vector to the cells of the BBB.

In one embodiment, the vector expresses a peptide on the vector surface that targets the vector to the cells of the BBB. In other words, the peptide, small molecule, antibody or antibody fragment thereof, protein, nanoparticle, lipid, oligonucleotide, aptamer, or cationic molecules expressed or comprised on the vector surface confers specificity of targeting to the cells of the BBB. In another embodiment, the peptide or other listed molecule expressed or comprised on the vector surface targets the vector to particular cell-type(s) of the BBB. Examples of suitable peptides, as well as methods for generating and testing such peptides including phage display methods, are known in the art. For example, the peptide can comprise a ligand or receptor-targeting peptide involved in cellular transcytosis. Such peptides allow uptake of the vector into the cells of the BBB using receptor-mediated transcytosis. In one embodiment, the peptide targets a receptor selected from:
transferrin receptor, insulin receptor and low-density lipoprotein receptor. In a further embodiment, the peptide comprises a transferrin peptide. An example BBB targeting peptide is the NRGTEWD (SEQ ID NO: 15) peptide included in the AAV2 strain, AAV-BR1 peptides such as these can be incorporated in non-viral vectors in some embodiments.

Thus, the vector may carry mutations, such as insertions, deletions or substitutions in vector surface proteins, such as viral capsid proteins, that result in targeting of the vector to the cells of the BBB. In a particular embodiment, the vector may comprise mutations that targets the vector to the cells of the BBB.

In the alternative or as an addition, a neurotropic viral vector comprising polynucleotide(s) encoding an antibody or antibody fragment may be used to selectively target cells of the BBB.

The term “vector” is well known in the art, and in the context of the invention is suitably used to transport (by transduction or transfection) a polynucleotide(s) into a host cell. This definition includes both non-viral and viral vectors. The viral or non-viral vectors may target cells from the BBB or CNS, such as but not limited to brain endothelial cells, to locally produce the therapeutic antibody molecule into the CNS, preferably into the brain parenchyma. Antibodies, in particular therapeutic antibodies, produced by this novel approach may be used to treat CNS-related disorders. In such a scenario, brain endothelial cells and/or pericytes and/or astrocytes act as a reservoir to provide high quality and long-term expression of antibodies into the CNS, preferably into the brain parenchyma.

Non-viral vectors include, but are not limited to, organic nanomaterials such as, liposomes, exosomes, dendrimers, and micelles or inorganic nanomaterials such as gold nanoparticles, silica nanoparticles and carbon nanotubes. In one embodiment, the non-viral vectors express a peptide, small molecule (SME), antibody or antibody fragment thereof, protein, nanoparticle, lipid, oligonucleotide, aptamer or cationic molecule on the vector surface that targets the vector to the cells of the BBB.

Viral vectors include, but are not limited to, wild-type viruses and engineered (e.g. modified) viruses. Examples of viral vectors include, but are not limited, to adeno associated virus (AAV), adenovirus, retrovirus, rhinovirus, lentivirus, hepatitis, HSV and any virus-like particle. As is known in the art, virus-like particles (VLPs) are multiprotein structures that mimic the organization and conformation of authentic native viruses but lack the viral genome. The invention stems from the unexpected discovery that transduction of brain endothelial cells by AAV vectors leads to secretion of high quality and large quantity of an antibody by brain endothelial cells. Therefore, in one embodiment, the viral vector is an AAV. The AAV can be of any suitable serotypes, examples of which include, but are not limited to, AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12), or any other wild type serotypes or engineered AAVs. More specifically, the AAV can be selected from: AAV serotype 1 (AAV1), AAV serotype 2 (AAV2), AAV serotype 8 (AAV8), AAV serotype 9 (AAV9) and AAV serotype 10 (AAV10). In another embodiment, the viral vector is selected from: AAV2, AAV8 AAV9 and AAVrh.10 (AAV rhesus isolate10).

In a further embodiment, the viral vector is an engineered AAV. The engineered AAV may be an engineered AAV2, engineered AAV9, engineered AAV1 or engineered AAV10. In a preferred embodiment, the engineered AAV2 is AAV-BR1. In another preferred embodiment, the engineered AAV9 is AAV-S, AAV-F, AAV-PHP.eB or AAV9-PHP-V. In a further preferred embodiment, the engineered AAV1 is AAV1RX, AAV1R6 or AAV1R7. Further details of engineered AAV1 are provided in Albright B H et al. incorporated herein by reference [53, 54]. In a most preferred embodiment, the vector is AAV-BR1 or AAV9-PHP-V1 that are specific for BBB endothelial cells.

In a further embodiment, the vector comprises viral and non-viral elements. Virosomes are an example of a vector that comprises both viral and non-viral elements. A further example is viral vectors mixed with cationic lipids.

All vectors that are described herein comprise polynucleotide(s) encoding an antibody or antibody fragment. The polynucleotide may comprise DNA or RNA. The polynucleotide may, for example, comprise additional components to assist with expression (e.g. translation) of the sequence encoding an antibody or antibody fragment in the cells of the BBB. For example, the polynucleotide encoding an antibody or antibody fragment is comprised within an expression cassette. In one embodiment, the expression cassette comprises, consists essentially of or consists of a nucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10.

In a further embodiment, the expression cassette comprises a nucleotide sequence with at least 80% identity to a nucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In a further embodiment, the expression cassette comprises a nucleotide sequence with at least 85% identity to a nucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In a further embodiment, the expression cassette comprises a nucleotide sequence with at least 90% identity to a nucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In a further embodiment, the expression cassette comprises a nucleotide sequence with at least 91% identity to a nucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In a further embodiment, the expression cassette comprises a nucleotide sequence with at least 92% identity to a nucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In a further embodiment, the expression cassette comprises a nucleotide sequence with at least 93% identity to a nucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In a further embodiment, the expression cassette comprises a nucleotide sequence with at least 94% identity to a nucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In a further embodiment, the expression cassette comprises a nucleotide sequence with at least 95% identity to a nucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In a further embodiment, the expression cassette comprises a nucleotide sequence with at least 96% identity to a nucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In a further embodiment, the expression cassette comprises a nucleotide sequence with at least 97% identity to a nucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In a further embodiment, the expression cassette comprises a nucleotide sequence with at least 98% identity to a nucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In a further embodiment, the expression cassette comprises a nucleotide sequence with at least 99% identity to a nucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10.

As used herein “% identity” is used to describe the sequence similarity between two sequences, such as nucleotide sequences and amino acids sequences. This may be determined by comparing the two sequences aligned in an optimum manner and in which the nucleotide sequence to be compared can comprise additions or deletions with respect to the reference sequence for an optimum alignment between these two sequences. The percentage of identity is calculated by determining the number of identical positions for which the residue is identical between the two sequences, dividing this number of identical positions by the total number of positions in the comparison window and multiplying the result obtained by 100 in order to obtain the percentage of identity between these two sequences. For example, it is possible to use the BLAST program, “BLAST 2 sequences” (Tatusova et al, “Blast 2 sequences—a new tool for comparing protein and nucleotide sequences”, FEMS Microbiol Lett. 174:247-250) available on the site https://blast.ncbi.nlm.nih.gov/Blast.cgi, the parameters used being those given by default (in particular for the parameters “open gap penalty”: 5, and “extension gap penalty”: 2; the matrix chosen being, for example, the matrix “BLOSUM 62” proposed by the program), the percentage of identity between the two sequences to be compared being calculated directly by the program.

The expression cassette may comprise sequences providing or encoding one or more of, and preferably all of, a promoter operably linked to the polynucleotide encoding an antibody or antibody fragment, a ribosomal binding site, a start codon, a stop codon, and a transcription termination sequence. Suitably the expression cassette may further comprise a nucleic acid encoding a post-transcriptional regulatory element. Suitably the expression cassette may additionally comprise a nucleic acid encoding a polyA (polyadenylation) element.

As used herein, the phrase “promoter” refers to a region of DNA that generally is located upstream of a polynucleotide sequence (e.g. the polynucleotide sequence encoding the antibody or antibody fragment) to be transcribed that is needed for transcription to occur, e.g. which initiates transcription. In some embodiments, the promoter is selected from: cytomegalovirus (CMV) promoter, EF1A (Human Eukaryotic translation elongation factor 1 alpha 1), CAG (CMV early enhancer fused to modified chicken β-actin promoter), CBh (CMV early enhancer fused to modified chicken β-actin promoter), SV40 (Simian virus 40 enhancer/early promoter), GFAP (Human glial fibrillary acidic protein promoter), ATP1A2_1 (Na, K ATPase α2), CLDN_5 (Claudin 5), ADRB2_1 (Adrenoceptor beta 2), TNFRSF6B_1 (TNF receptor superfamily member 6b), PDYN_1 (prodynorphin), GH1_1 (Human growth hormone), OPALIN_1 (Opalin), SYN1_1 (Synapsin 1), CAMK2A_1 (Calcium/Calmodulin Dependent Protein Kinase II alpha), NEFH_1 (neurofilament heavy polypeptide), NEUROD6_1 (neuronal differentiation factor 6) or OLIG2_1 (oligodendrocyte transcription factor 2). In a preferred embodiment, the promoter is a cytomegalovirus (CMV) promoter, a CBh, a CMV early enhancer fused to either GFAP, ATP1A2_1, CLDN_5, ADRB2_1, TNFRSF6B_1, PDYN_1, GH1_1. OPALIN_1, SYN1_1, CAMK2A_1, NEFH_1, NEUROD6_1 or OLIG2_1 promoter. In a more preferred embodiment, the promoter is a CBh, CMV, or a CMV early enhancer fused to either GFAP or OLIG2. In an even more preferred embodiment, the promoter is a CMV promoter or CBh promoter.

The term promoter includes synthetic promoters. The term “synthetic promoter” as used herein relates to a promoter that does not occur in nature. For example, functional variants of naturally occurring promoters can be used in accordance with the invention. A “functional variant” of a promoter in the context of the present invention is a variant of a reference promoter that retains the ability to function in the same way as the reference promoter. In additional embodiments, truncated forms of naturally occurring promoters are used. In preferred embodiments, the promoter is operably linked to an enhancer such as the CMV early enhancer. Truncated or modified naturally occurring promoters can be used to facilitate insertion of relatively large antibody encoding sequences into a vector, in particular a viral vector.

As described in detail elsewhere, the vector may specifically target cells of the BBB (and/or the CNS). However, in other instances the vectors do not specifically target the BBB (and/or the CNS). For example, many wild-type viral vectors target any tissue or cell-type. In such cases, BBB-specific or CNS-specific promoters can be used to drive the expression of the polynucleotide encoding an antibody or antibody fragment in the cells of the BBB or CNS in a preferential or predominant manner as compared to other tissues.

In one embodiment, the polynucleotide comprises a GFAP (Human glial fibrillary acidic protein) promoter operably linked to a polynucleotide encoding an antibody or antibody fragment. In a further embodiment, the GFAP promoter is operably linked to a CMV early enhancer. In this instance there is preferential or predominant expression of the antibody or antibody fragment in astrocytes.

In one embodiment, the polynucleotide comprises a promoter selected from: ATP1A2_1 (Na, K ATPase α2), CLDN_5 (Claudin 5), ADRB2_1 (Adrenoceptor beta 2) and TNFRSF6B_1 (TNF receptor superfamily member 6b) operably linked to a polynucleotide encoding an antibody or antibody fragment. In a further embodiment, the promoter is operably linked to a CMV early enhancer. In this instance there is preferential or predominant expression of the antibody or antibody fragment in endothelial cells of the BBB.

In one embodiment, the polynucleotide comprises a promoter selected from: PDYN_1 (prodynorphin), GH1_1 (Human growth hormone) and OPALIN_1 (Opalin) operably linked to a polynucleotide encoding an antibody or antibody fragment. In a further embodiment, the promoter is operably linked to a CMV early enhancer. In this instance there is preferential or predominant expression of the antibody or antibody fragment in brain cells.

In one embodiment, the polynucleotide comprises a promoter selected from: SYN1_1 (Synapsin 1), CAMK2A_1 (Calcium/Calmodulin Dependent Protein Kinase II alpha), NEFH_1 (neurofilament heavy polypeptide), NEUROD6_1 (neuronal differentiation factor 6) operably linked to a polynucleotide encoding an antibody or antibody fragment. In a further embodiment, the promoter is operably linked to a CMV early enhancer. In this instance there is preferential or predominant expression of the antibody or antibody fragment in neurons.

In one embodiment, the polynucleotide comprises an OLIG2_1 (oligodendrocyte transcription factor 2) promoter operably linked to a polynucleotide encoding an antibody or antibody fragment. In a further embodiment, the OLIG2_1 promoter is operably linked to a CMV early enhancer. In this instance there is preferential or predominant expression of the antibody or antibody fragment in oligodendrocytes.

The term “operably linked” as used herein refer to the arrangement of various polynucleotide elements relative to each such that the elements are functionally connected and are able to interact with each other in the manner intended. Such elements may include, without limitation, a promoter, an enhancer and/or a regulatory element, a polyadenylation sequence, one or more introns and/or exons, and a coding sequence of a gene of interest to be expressed. The polynucleotide elements, when properly oriented or operably linked, act together to modulate the activity of one another, and ultimately may affect the level of expression of a product (e.g. an antibody or antibody fragment). By modulate is meant increasing, decreasing, or maintaining the level of activity of a particular element. As understood by a person of average skill in the art, operably linked implies functional activity, and is not necessarily related to a natural positional link.

As described above, the expression cassette may comprise sequences providing or coding for a ribosomal binding site (RBS). In a preferred embodiment, the RBS is an internal ribosome entry site (IRES). In one embodiment, the IRES is derived from encephalomyocarditis virus. In a further embodiment, the IRES comprises SEQ ID NO: 1 or SEQ ID NO: 8. As IRES is especially advantageous in an expression cassette comprising more than one gene encoding a antibody or antibody fragment. For example, wherein the expression cassette comprises a gene encoding a light chain of an antibody or antibody fragment and a gene encoding a heavy chain of the antibody or antibody fragment.

According to all aspects of the invention, the expression cassette may in addition comprise sequences providing or coding for a Woodchuck Hepatitis Virus Posttranscriptional Regulatory Element (WPRE) at the 3′ end of the construct as represented diagrammatically in topmost construct of FIG. 1. The WPRE sequence is routinely used to increase expression of genes delivered by viral vectors. Without wishing to be bound by theory, inclusion of the sequence in expression cassettes can increase mRNA stability and thus protein yield.

In the alternative, the expression cassette may comprise sequences encoding for self-cleavage peptides. Said self-cleavage peptides can be used in an expression cassette comprising more than one gene encoding an antibody or antibody fragment. For example, wherein the expression cassette comprises a single promoter operably linked to a gene encoding a light chain of an antibody or antibody fragment and to a gene encoding a heavy chain of the antibody or antibody fragment. In such instances, the sequences encoding for self-cleavage peptides are located after the first gene (e.g. the gene encoding a light chain of an antibody or antibody fragment) and before the second gene (e.g. the gene encoding a heavy chain of an antibody or antibody fragment). Such systems may allow for self-cleavage of a peptide to occur co-translationally via ribosomal skipping (e.g. which results in separate heavy and light chain polypeptides of the antibody or antibody fragment from a single mRNA transcript). One particular class of self-cleavage peptides is the 2A peptide family (including the F2A peptide which was derived from foot-and-mouth disease virus) which share a core sequence motif of DxExNPGP (SEQ ID NO: 16). Therefore, in one embodiment the expression cassette comprises a sequence encoding a self-cleavage peptide from the 2A family In further embodiments, the expression cassette further comprises sequences encoding for a furin cleavage site upstream of the self-cleavage site. In other words, the expression cassette comprises a single promoter operably linked to a gene encoding a light chain of an antibody or antibody fragment and to a gene encoding a heavy chain of the antibody or antibody fragment, wherein the sequences encoding for the furin cleavage peptide and the self-cleavage peptides are located after the first gene and before the second gene. The addition of a furin cleavage site can be made to eliminate additional amino acids of the self-cleavage peptide that would otherwise remain attached to the upstream protein (e.g. the light chain of an antibody or antibody fragment) after self-cleavage. It is noteworthy, however, that even with a furin cleavage site upstream of the self-cleavage peptide that additional amino acids may remain in some of the upstream protein (e.g. the light chain of an antibody or antibody fragment) and that this can lead to an immune response (e.g. immunogenicity) to the upstream protein. Moreover, the inventors have surprisingly observed increased aggregation of constructs containing furin and 2A to allow self-cleavage. Therefore, in a preferred embodiment the expression cassette does not comprise sequences encoding self-cleavage peptides after a first gene and before a second gene.

The expression cassette may further comprise a secretion peptide at the 5′ terminus of the polynucleotide encoding an antibody or antibody fragment. In the context of this invention the secretion peptide assists with delivery of the antibody or antibody fragment into the CNS, preferably into the brain parenchyma. In instances in which there are multiple genes encoding the antibody or antibody fragment, all genes may further comprise a sequence encoding a secretion peptide.

The invention is not limited to a specific polynucleotide encoding a particular antibody or antibody fragment. Typically, however, the antibody or antibody fragment is a therapeutic antibody or antibody fragment. The therapeutic antibody or antibody fragment is one that usefully exerts its activity in the CNS, in particular in the brain. Thus, the therapeutic antibody or antibody fragment may bind to a target antigen expressed in the CNS, in particular the brain or spinal cord.

In general, the term “antibody” is used herein in the broadest sense and encompasses various antibody structures, including but not limited to monoclonal antibodies, polyclonal antibodies, multispecific antibodies (e.g., bispecific antibodies), fully human antibodies and antibody fragments so long as they exhibit the desired antigen-binding activity. The antibodies may also be chimeric antibodies (especially mouse VH and VL regions fused with human constant domains), recombinant antibodies, antigen-binding fragments of recombinant antibodies, humanized antibodies.

An “antibody fragment” of an antibody refers to a molecule other than an intact antibody that comprises a portion of an intact antibody and that binds the antigen to which the intact antibody binds. In one embodiment, the antibody fragments is Fv, Fab, Fab′, Fab′-SH, F(ab′)2; diabodies; linear antibodies; single-chain antibody molecules (e.g. scFv and preferably scFv); and multispecific antibodies formed from antibody fragments. The term also encompasses single domain antibodies (e.g. VHH, VNARs or human single domain antibody). One preferred antibody fragment according to the invention is scFv-Fc, in which an scFv (a fusion protein of VH and VL domains connected with a short linker peptide, typically of around 10-25 amino acids) is joined with the fragment crystallizable (Fc) region. The scFv-Fc lacks the CH1 and CL domains.

The term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modified “monoclonal” indicates the character of the antibody as being amongst a substantially homogeneous population of antibodies and is not to be construed as requiring production of the antibody by any particular method. Monoclonal antibodies to be used in accordance with the present invention may be made by the hybridoma method described by Kohler, Nature 256 (1975), 495.

Accordingly, in the context of the present invention, the term “antibody” relates to full immunoglobulin molecules as well as to parts of such immunoglobulin molecules (i.e., “antibody fragment”). Furthermore, the term relates, as discussed above, to modified and/or altered antibody molecules. The term also relates to recombinantly or synthetically generated/synthesized antibodies. The term also relates to intact antibodies as well as to antibody fragments thereof, like, separated light and heavy chains, Fab, Fv, Fab′, Fab′-SH, F(ab′)2. The term “antibody” also comprises but is not limited to fully human antibodies, chimeric antibodies, humanized antibodies, CDR-grafted antibodies and antibody constructs, like single chain Fvs (scFv), scFv-Fcs or antibody-fusion proteins.

Humanized antibodies are modified antibodies that are also referred to as reshaped human antibodies. A humanized antibody is constructed by transferring the CDRs of an antibody derived from an immunized animal to the complementarity determining regions of a human antibody. Conventional genetic recombination techniques for such purposes are known (see European Patent Application Publication No. EP 239400; International Publication No. WO 96/02576; Sato K. et al., Cancer Research 1993, 53: 851-856; International Publication No. WO 99/51743).

The term “CDR” as employed herein relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. VH-CDR, or CDR-H depicts a CDR region of a variable heavy chain and VL-CDR, or CDR-L relates to a CDR region of a variable light chain. VH means the variable heavy chain and VL means the variable light chain. The CDR regions of an Ig-derived region may be determined as described in Kabat “Sequences of Proteins of Immunological Interest”, 5th edit. NIH Publication no. 91-3242 U.S. Department of Health and Human Services (1991); Chothia J., Mol. Biol. 196 (1987), 901-917 or Chothia, Nature 342 (1989), 877-883.

An “Fe” region contains two heavy chain fragments comprising the CH2 and CH3 domains of an antibody. The two heavy chain fragments are held together by two or more disulfide bonds and by hydrophobic interactions of the CH3 domains.

A “Fab′ fragment” contains one light chain and a portion of one heavy chain that contains the VH domain and the CH1 domain and also the region between the CH1 and CH2 domains, such that an interchain disulfide bond can be formed between the two heavy chains of two Fab′ fragments to form a F(ab′) 2 molecule.

A “F(ab′)2 fragment” contains two light chains and two heavy chains containing a portion of the constant region between the CH1 and CH2 domains, such that an interchain disulfide bond is formed between the two heavy chains. A F(ab′)2 fragment thus is composed of two Fab′ fragments that are held together by a disulfide bond between the two heavy chains.

The “Fv region” comprises the variable regions from both the heavy and light chains, but lacks the constant regions.

The “scFv-Fc” comprises the “Fv” variable regions from both the heavy and light chains, fused to an “Fc” region containing two heavy chain fragments comprising the CH2 and CH3 domains of an antibody. The two heavy chain fragments are held together by two or more disulfide bonds and by hydrophobic interactions of the CH3 domains.

Accordingly, in the context of this invention, antibody molecules or antibody fragments thereof are provided, which are humanized and can successfully be employed in pharmaceutical compositions, including mixtures of at least two antibody molecules or antibody fragments thereof.

An “antibody that binds to an epitope” within a defined region of a protein is an antibody that requires the presence of one or more of the amino acids within that region for binding to the protein.

In certain embodiments, an “antibody that binds to an epitope” within a defined region of a protein is identified by mutation analysis, in which amino acids of the protein are mutated, and binding of the antibody to the resulting altered protein (e.g., an altered protein comprising the epitope) is determined to be at least 20% of the binding to unaltered protein. In some embodiments, an “antibody that binds to an epitope” within a defined region of a protein is identified by mutation analysis, in which amino acids of the protein are mutated, and binding of the antibody to the resulting altered protein (e.g., an altered protein comprising the epitope) is determined to be at least 30%, at least 40%, at least 50%, at least 60%, at least 70%, at least 80%, or at least 90% of the binding to unaltered protein. In certain embodiments, binding of the antibody is determined by FACS, WB or by a suitable binding assay such as ELISA.

The antibody or antibody fragment for use in accordance with the invention is preferably an antibody or antibody fragment that binds to an epitope in the CNS. More specifically, the antibody or antibody fragment binds to an epitope in the CNS that is associated with a disease or disorder of the CNS. In a preferred embodiment, the antibody or antibody fragment is selected from an: an: anti-ErbB2, anti-TDP-43 (NI-205), anti-Abeta (such as bapineuzumab, solanezumab, lecanemab, aducanumab, donanemab, gantenerumab or crenezumab), anti-ApoE4 (Apolipoprotein E4) and anti-DDX3X (ATP-dependent RNA helicase), anti-Tau (tilavonemab, gosuranemab, zagotenemab, semorinemab, bepranemab, BIIB076, JNJ-63733657, Lu AF87908, PNT001, E-2814), anti-LINGO-1 (such as opicinumab), anti-alpha-synuclein (cinpanemab, prasinezumab, MEDI-1341, Lu AF82422, BAN0805), anti-ASC (IC-100), anti-NLRP3, anti-C5 (ravulizumab, eculizumab), anti-Clq (ANX-005), anti-C3, anti-huntingtin (C-617, NI-302), anti-prion, anti-CD20 (such as ofatumumab, ocrelizumab, rituximab, BCD-132, ublituximab, BAT-4406F, AL-014), anti-PD-1 (IBC-Ab002) or anti-VEGF-A (bevacizumab, ranibizumab, brolucizumab, faricimab, vanucizumab).

As explained in the experimental section herein, human brain endothelial cell line hCMEC/D3 were transduced by AAV2, AAV8, AAV9 and AAVrh.10 (AAV rhesus isolate10) delivering the gene coding for one of: anti-TDP-43 antibody MAB1 (chimeric human IgG1), anti-ErbB2 antibody (Herceptin) MAB2 or eGFP (enhanced green fluorescent protein). This resulted in high quality and long-term expression of antibodies over time. The inventors have also confirmed their observations in mouse cell lines and human primary brain endothelial cells. The delivery system resulted in the production of correctly folded antibodies in a range of different antibody formats (including full-length antibodies and antibody fragments). Critically, in these experiments the secreted antibodies were detected in both the apical and basolateral sides, confirming delivery to the brain parenchyma side of the BBB in this model. As such, the data contained in the examples indicates that the methods and vectors described herein can be used to deliver correctly folded, antibodies or antibody fragments (such as therapeutic antibodies) to the CNS.

It is an object of the present invention to provide for the first time a method to increase antibody concentration in the CNS in order to treat CNS-related diseases or disorders by delivering polynucleotide(s) encoding an antibody or antibody fragment to cells in the CNS and/or BBB. This novel approach can be useful to treat various diseases or disorders that originate in the CNS.

For the avoidance of doubt, as used herein the term “treatment” includes the therapeutic treatment, as well as the symptomatic treatment and the prophylaxis of a condition. Use of the term “treat”, “treating”, or “treatment of” (and grammatical variations thereof) means that the severity of a subject's condition is reduced, at least partially improved, or ameliorated and/or that some alleviation, mitigation or decrease in at least one clinical symptom is achieved and/or there is a delay in the progression of the disease or disorder.

As used here in the term “subject” refers to an individual, e.g., a mammal such as a human, having or at risk of having a specified condition, disorder or symptom present within the CNS. The subject may be a subject in need of treatment in accordance with the invention. The subject may have received treatment for the condition, disorder or symptom. Alternatively, the subject has not been treated prior to treatment in accordance with the present invention.

The invention relates to an innovative strategy that can be used in any mammal, including human subjects, and employed for the treatment of diseases or disorders associated in a subject who is suffering from a disease or disorder of the CNS. By delivering therapeutic antibody genes into brain endothelial cells, they act as reservoir to provide high quality and long-term expression of antibodies into the CNS.

In one embodiment, the disease or disorder is a neurodegenerative disorder.

In a further embodiment, the disease or disorder is associated with the CNS, including but not limited to diseases associated with amyloid-beta protein, TDP-43-proteinopathies, alpha-synucleinopathies, Tauopathies, trinucleotide repeat disorders including poly-glutamine disorders such as Huntington's disease, brain-related cancers and tumors, epilepsy, psychiatric diseases, neuroinflammatory diseases, neuromuscular diseases, viral-induced encephalitis and diseases characterized by microglial dysfunction.

In another embodiment, the disease or disorder or conditions associated of a patient who is a disease associated with amyloid-beta protein, TDP-43-proteinopathies, alpha-synucleinopathies, Tauopathies, trinucleotide repeat disorders including poly-glutamine disorders such as Huntington's disease, brain-related cancers and tumors, epilepsy, psychiatric diseases, neuroinflammatory diseases, neuromuscular diseases, viral-induced encephalitis, and diseases characterized by microglial dysfunction.

In some embodiments, the amyloid-beta associated diseases, disorders or conditions according to the invention include mild cognitive impairment (MCI), Down syndrome (DS), Down syndrome-related Alzheimer's Disease, cerebral amyloid angiopathy (CAA), multiple sclerosis, Parkinson's disease (PD), Parkinson's Disease with Dementia (PDD), Dementia with Lewy Body, ALS (amyotrophic lateral sclerosis). Many of these conditions are characterized by, or associated with, loss of cognitive memory capacity. Conditions characterized by, or associated with, loss of cognitive memory capacity according to the invention therefore include AD, mild cognitive impairment (MCI), Down syndrome (DS), Down syndrome-related Alzheimer's Disease, cerebral amyloid angiopathy (CAA), multiple sclerosis, Parkinson's disease, Parkinson's disease with Dementia (PDD), Dementia with Lewy body, ALS (amyotrophic lateral sclerosis).

Particularly, the amyloid-beta associated diseases, disorders or conditions may be selected from Alzheimer's Disease (AD), Down syndrome (DS), Down syndrome-related Alzheimer's Disease, cerebral amyloid angiopathy (CAA), or Lewy body dementia.

In some embodiments, the TDP-43-proteinopathies include Frontotemporal dementia (FTD, such as Sporadic or familial with or without motor-neuron disease (MND), with progranulin (GRN) mutation, with C9orf72 mutations, with TARDBP mutation, with valosine-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant primary progressive aphasia (svPPA), behavioural variant FTD (bvFTD), Nonfluent Variant Primary Progressive Aphasia (nfvPPA) and the like), Amyotrophic lateral sclerosis (ALS, such as Sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known as Machado Joseph Disease)), Hippocampal sclerosis dementia and Myopathies (Sporadic inclusion body myositis, Inclusion body myopathy with a mutation in the valosin-containing protein (VCP; also Paget disease of bone and frontotemporal dementia)), Traumatic Brain Injury (TBI), Dementia with Lewy Bodies (DLB) or Parkinson's Disease (PD).

Particularly, the TDP-43-proteinopathies diseases, disorders or conditions may be selected from Frontotemporal dementia (FTD), amyotrophic lateral sclerosis (ALS), Alzheimer's disease (AD), Parkinson's disease (PD), Chronic Traumatic Encephalopathy (CTE), limbic-predominant age-related TDP-43 encephalopathy (LATE) and multiple sclerosis (MS).

In some embodiments, the synucleinopathy is Parkinson's disease (sporadic, familial with alpha-synuclein mutations, familial with mutations other than alpha-synuclein, pure autonomic failure and Lewy body dysphagia), Lewy Body dementia (LBD; including dementia with Lewy bodies (DLB) (“pure” Lewy body dementia), Parkinson's disease dementia (PDD)), or Diffuse Lewy Body Disease, sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (Shy-Drager syndrome, striatonigral degeneration and olivopontocerebellar atrophy), traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectasia, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder.

Particularly, the synucleinopathy may be selected from Parkinson's Disease, Multiple System Atrophy, Lewy Body dementia (LBD; including dementia with Lewy bodies (DLB) (“pure” Lewy body dementia), Parkinson's disease dementia (PDD)), or Diffuse Lewy Body Disease.

In some embodiments, the tauopathy is selected from Alzheimer's Disease, amyotrophic lateral sclerosis, Parkinson's disease, Creutzfeldt-Jacob disease, Dementia pugilistica, Down's Syndrome, Gerstmann-Straussler-Scheinker disease, prion protein cerebral amyloid angiopathy, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotetemporal dementia, frontotemporal dementia with parkinsonism linked to chromosome 17, Hallervorden-Spatz disease, multiple system atrophy, Niemann-Pick disease type C, Pallido-ponto-nigral degeneration, Pick's disease, progressive subcortical gliosis, progressive supranuclear palsy, Subacute sclerosing panencephalitis, Tangle only dementia, Postencephalitic Parkinsonism, and Myotonic dystrophy.

Particularly, the tauopathy may be selected from Alzheimer's disease or progressive supranuclear palsy.

In some embodiments, the neuroinflammatory diseases, disorders or abnormalities are selected from Alzheimer's disease, Parkinson's disease, Frontotemporal dementia, limbic-predominant age-related TDP-43 encephalopathy, amyotrophic lateral sclerosis, motor neuron disease, trinucleotide repeat disorders including poly-glutamine disorders such as Huntington's disease, multiple sclerosis, demyelination, viral encephalitis, epilepsy, ischemic and hemorrhagic stroke, traumatic brain injury, chronic traumatic encephalopathy, cryopyrin-associated periodic syndromes (CAPS), Muckle-Wells syndrome (MWS), familial cold autoinflammatory syndrome (FCAS), neonatal-onset multisystem inflammatory disease (NOMID), periodic fever syndrome (HIDS), sideroblastic anemia with B-cell immunodeficiency, periodic fevers, developmental delay (SIFD), Behçet's disease, Sjogren's syndrome, cerebral malaria, brain injury from pneumococcal meningitis, Chikungunya virus, Ross River virus, influenza, HIV, Coronaviruses, Dengue, Zika virus, helminth infection, bacterial infection, depression, psychological stress, HIV-associated neurocognitive disorder, Coronavirus-associated inflammatory pathologies.

In some embodiments, the neuroinflammatory diseases, disorders or abnormalities are preferably selected from Alzheimer's disease, Parkinson's disease, amyotrophic lateral sclerosis, frontotemporal dementia, multiple sclerosis, demyelination, viral encephalitis, epilepsy, stroke, cryopyrin-associated periodic syndromes (CAPS), anti-neutrophil cytoplasmic antibody-associated vasculitis, lupus, Psoriatic Arthritis, and Hereditary Recurrent Fevers (HRFs).

In some embodiments, the neuroinflammation diseases, disorders or abnormalities are more preferably selected from Alzheimer's disease, Parkinson's disease, amyotrophic lateral sclerosis, frontotemporal dementia, multiple sclerosis, demyelination, viral encephalitis, stroke, cryopyrin-associated periodic syndromes (CAPS).

In some embodiments, neuromuscular diseases may include cerebrovascular accident, Parkinson's disease, multiple sclerosis, Huntington's disease and Creutzfeldt—Jakob disease, Spinal muscular atrophies and amyotrophic lateral sclerosis. In another embodiment, the CNS disease or disorder is a neurodegenerative disorder.

In a preferred embodiment, the disease or disorder of the CNS is selected from: Frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE) and multiple sclerosis (MS).

Brain and CNS cancers and tumors that may also be treated in accordance with the invention include astrocytomas (including cerebellar and cerebral), brain stem glioma, brain tumors, malignant gliomas, ependymoma, glioblastoma, medulloblastoma, supratentorial primitive neuroectodermal tumors, visual pathway and hypothalamic gliomas, primary central nervous system lymphoma, ependymoma, brain stem glioma, visual pathway and hypothalamic glioma, extracranial germ cell tumor, medulloblastoma, myelodysplasia syndromes, oligodendroglioma, myelodysplastic/myeloproliferative diseases, myelogenous leukemia, myeloid leukemia, multiple myeloma, myeloproliferative disorders, neuroblastoma, plasma cell neoplasm/multiple myeloma, central nervous system lymphoma, intrinsic brain tumors, astrocytic brain tumors, gliomas, and metastatic tumor cell invasion in the central nervous system.

The vectors as described herein can be administered to the subject by any conventional route, including injection or by gradual infusion over time. The administration may be via parenteral administration. The administration may, for example, be by infusion or by intrathecal, intra-cisternal, intracerebroventricular, intraparenchymal, intranasal, intravitreous, subcutaneous, or intramuscular route. In one embodiment, the vector is administered by intravenous injection or intravenous infusion. As further examples, suitable forms for parenteral injection (including, subcutaneous, intramuscular, intravascular or infusion) include a sterile solution, suspension or emulsion. Intravenous injection is preferred.

The identification of suitable dosages of the compositions of the invention is well within the routine capabilities of a person of average skill in the art. For example, the suitable dosage for a given subject will be determined by taking into consideration various factors known to modify the action of the vector for the use according to the invention. For example, severity and type of CNS disease or disorder, body weight, sex, diet, time and route of administration, other medications and other relevant clinical factors. The dosages and schedules may be varied according to the particular condition, disorder or symptom the overall condition of the subject. It may also be the case that there is no single accepted dose for the treatment of a given disease, but that a range of closes is considered suitable. Effective dosages may be determined by either in vitro or in vivo methods.

Also provided herein are methods for treating a disease or disorder of the CNS in a subject. The method comprises administering a vector comprising a polynucleotide encoding an antibody or antibody fragment to the subject. Said method results in transduction or transfection of cells of the BBB, and advantageously the transduced or transfected cells produce (e.g. express) the antibody or antibody fragment and the antibody or antibody fragment is delivered into the CNS, preferably into the brain parenchyma.

In another aspect, the invention provides a method for delivery of an antibody or antibody fragment to the BBB in a subject, the method comprising administering a vector comprising a polynucleotide encoding the antibody or antibody fragment to the subject, wherein the method results in transduction or transfection of cells of the BBB and the transduced or transfected cells express the antibody or antibody fragment.

In a further aspect, the invention provides a method for delivery of an antibody or antibody fragment to the CNS in a subject, the method comprising administering a vector comprising a polynucleotide encoding the antibody or antibody fragment to the subject, wherein the method results in transduction or transfection of cells of the BBB and the transduced or transfected cells express the antibody or antibody fragment resulting in delivery of the antibody or antibody fragment into the CNS.

In a still further aspect, the invention provides a method for treating a disease or disorder of the CNS in a subject, the method comprising administering a vector comprising a polynucleotide encoding an antibody or antibody fragment to the subject, wherein the method results in transduction or transfection of cells of the BBB and the transduced or transfected cells express the antibody or antibody fragment resulting in delivery of the antibody or antibody fragment into the CNS.

In another aspect, the invention provides for the use of a vector comprising a polynucleotide encoding an antibody or antibody fragment for the manufacture of a medicament for the treatment of a disease or disorder of the CNS in a subject, wherein the vector transduces or transfects cells of the blood brain barrier (BBB) and the transduced or transfected cells express the antibody or antibody fragment resulting in delivery of the antibody or antibody fragment into the CNS.

In a further aspect, the invention provides for the use of a vector comprising a polynucleotide encoding an antibody or antibody fragment for delivery of the polynucleotide encoding the antibody or antibody fragment to the BBB of a subject wherein the vector transduces or transfects cells of the blood brain barrier (BBB) and the transduced or transfected cells express the antibody or antibody fragment resulting in delivery of the antibody or antibody fragment into the CNS.

All embodiments described herein relating to vectors for use according to the preceding aspects of the invention are equally applicable to these further aspects of the invention.

The inventors have discovered that particular expression cassette constructs improve the quality of antibodies produced. This is important when delivering antibodies in vivo. Thus, using the expression cassettes of the invention (as detailed in the various aspects and embodiments of the invention herein) provides advantageous delivery of antibodies and antibody fragments to the CNS, which may be by direct delivery to the CNS or by delivery via BBB cells (according to the invention as defined herein). The aspects below may therefore be performed in vivo, in which the cells are BBB cells, especially brain endothelial cells or more generally cells of the CNS. Thus, the antibody or antibody fragment is generally a therapeutic antibody or antibody fragment, as described herein. The aspects below may also be performed ex vivo or in vitro for antibody production in some embodiments. In those embodiments, any suitable cell type may be utilized. Here, again, the antibody or antibody fragment is generally a therapeutic antibody or antibody fragment.

Accordingly, the invention provides a vector comprising an expression cassette comprising from 5′ to 3′: at least one promoter operably linked to a first gene encoding a light chain of an antibody or antibody fragment and to a second gene encoding a heavy chain of the antibody or antibody fragment for use in a method of treatment of a disease or disorder of the central nervous system (CNS) in a subject, wherein the vector transduces or transfects cells of the blood brain barrier (BBB) or the CNS and the transduced or transfected cells express the antibody or antibody fragment resulting in delivery of the antibody or antibody fragment into the CNS preferably into the brain parenchyma. The inventors have discovered that ordering the light chain before the heavy chain in such constructs is significantly advantageous. Preferably an IRES is positioned between the first and second genes.

In a further aspect, the invention provides a vector comprising an expression cassette comprising from 5′ to 3′: a first promoter operably linked to a first gene encoding a light chain of an antibody or antibody fragment and a second promoter operably linked to a second gene encoding a heavy chain of the antibody or antibody fragment for use in a method of treatment of a disease or disorder of the central nervous system (CNS) in a subject, wherein the vector transduces or transfects cells of the blood brain barrier (BBB) or the CNS and the transduced or transfected cells express the antibody or antibody fragment resulting in delivery of the antibody or antibody fragment into the CNS, preferably into the brain parenchyma. In such a construct, no IRES is needed.

The CNS, as with the BBB, comprises multiple cell-types and in one embodiment the vector transduces or transfects the polynucleotides(s) encoding an antibody or antibody fragment into at least one (up to all) cell-type in the CNS. For example, the vector may transduce or transfect cells of the CNS that are selected from: brain endothelial cells, neurons, pericytes, astrocytes, oligodendrocytes, microglia and ependymal cells. Thus, the vector may transduce or transfect neuronal and non-neuronal (glial) cells of the CNS. Alternatively, the vector may transduce or transfect one particular cell-type of the CNS. For example, the vector may transduce or transfect brain endothelial cells of the CNS. In another example, the vector may transduce or transfect the neurons of the CNS. In a further example, the vector may transduce or transfect the astrocytes of the CNS. In yet another example, the vector may transduce or transfect oligodendrocytes. In a yet further example, the vector may transduce or transfect microglia of the CNS. Alternatively, the vector may transduce or transfect ependymal cells of the CNS.

In a further embodiment, the vector transduces or transfects a plurality of cell-types from both the BBB and the CNS.

As described above, the vector comprising polynucleotide(s) encoding an antibody or antibody fragment may transduce or transfect, in particular, cells of the BBB and/or CNS. The same techniques as those described for targeting to the BBB described elsewhere are equally applicable to vector targeting directly to the CNS. In one embodiment, the vector expresses a peptide on the vector surface that targets the vector to the cells of the BBB or the CNS. In other words, the peptide expressed on the vector surface confers specificity of targeting to the BBB and/or CNS. In another embodiment, the peptide expressed on the vector surface targets the vector to particular cell-type(s) of the BBB and/or the CNS. Examples of suitable peptides, as well as methods for generating and testing such peptides including phage display methods, are known in the art.

In the alternative or as an addition, a neurotropic vector comprising polynucleotide(s) encoding an antibody or antibody fragment may be used to transduce or transfect, in particular, cells of the BBB and/or CNS such as HSV. Therefore, in one embodiment, the vector comprises a neurotropic vector. In another embodiment, the vector comprises a modified HSV.

All embodiments described herein relating to vectors for use according to the preceding aspects of the invention are equally applicable to these further aspects of the invention.

In one embodiment, the expression cassette comprises from 5′ to 3′: at least one promoter operably linked to a first gene encoding a light chain of an antibody or antibody fragment and to a second gene encoding a heavy chain of the antibody or antibody fragment. The expression cassette may further comprise an internal ribosomal entry site (IRES) after the first gene encoding the light chain of the antibody or antibody fragment and before the second gene encoding the heavy chain of the antibody. In other words, the expression cassette comprises from 5′ to 3′: at least one promoter operably linked to a first gene encoding a light chain of an antibody or antibody fragment, an IRES and a second gene encoding a heavy chain of the antibody or antibody fragment. In a further embodiment, the expression cassette comprises from 5′ to 3′: a first promoter operably linked to a first gene encoding a light chain of an antibody or antibody fragment and a second promoter operably linked to a second gene encoding a heavy chain of the antibody or antibody fragment. The position of each element within the expression cassette is relative to other elements and is expressed here in order starting at the 5′ end and moving towards the 3′ end as is customary.

The invention also relates to an unexpected higher antibody expression yield, and an improved protein quality by alternating the chain positions, using different secretion peptides, and customizing intra- and down-stream regulatory elements with respect to the previously reported strategies in the prior art.

Thus, the invention provides a method of reducing antibody or antibody fragment aggregation, wherein the method comprises:
(i) transforming cells with an expression cassette comprising from 5′ to 3′: at least one promoter, operably linked to a first gene encoding a light chain of an antibody or antibody fragment and to a second gene encoding a heavy chain of the antibody or antibody fragment and wherein the expression cassette further comprises an internal ribosomal entry site (IRES) after the first gene encoding the light chain of the antibody or antibody fragment and before the second gene encoding the heavy chain of the antibody or antibody fragment; or with an expression cassette comprising from 5′ to 3′: a first promoter operably linked to a first gene encoding a light chain of an antibody or antibody fragment and a second promoter operably linked to a second gene encoding a heavy chain of the antibody or antibody fragment; and
(ii) maintaining the transformed cells under conditions suitable for antibody or antibody fragment production.

The same constructs also result in improved antibody quality as compared to methods in which other constructs are employed (as discussed herein, in particular those with the heavy chain before the light chain in the cassette and/or incorporating self-cleavage peptides such as the furin/2A peptide). The invention therefore also provides a method of improving antibody or antibody fragment maturation and/or quality, wherein the method comprises:
(i) transforming cells with an expression cassette comprising from 5′ to 3′: at least one promoter, operably linked to a first gene encoding a light chain of an antibody or antibody fragment and to a second gene encoding a heavy chain of the antibody or antibody fragment and wherein the expression cassette further comprises an internal ribosomal entry site (IRES) after the first gene encoding the light chain of the antibody or antibody fragment and before the second gene encoding the heavy chain of the antibody or antibody fragment; or with an expression cassette comprising from 5′ to 3′: a first promoter operably linked to a first gene encoding a light chain of an antibody or antibody fragment and a second promoter operably linked to a second gene encoding a heavy chain of the antibody or antibody fragment; and
(ii) maintaining the transformed cells under conditions suitable for antibody or antibody fragment production.

In other words, one method in accordance with the invention results in an increase in the proportion of antibody or antibody fragment having the same three-dimensional structure as the native configuration of the antibody or antibody fragment. This may be measured for example using electrophoresis, such as SDS-PAGE. A sample may also be reduced (e.g. using DTT) and then run on a gel to confirm accurate disulfide bond formation and that both the light and heavy chain migrate at the expected molecular weight.

The inventors have surprisingly observed that the positioning of the gene encoding the light chain of an antibody or an antibody fragment relative to the gene encoding heavy chain of the antibody or the antibody fragment impacts the proportion of aggregation observed. Indeed, when the gene encoding the light chain is the first gene in the expression cassette followed by the gene encoding the heavy chain there is reduced aggregation of the expressed antibody as compared to the reverse orientation (e.g. when the gene encoding the heavy chain is the first gene in the expression cassette followed by the gene encoding the light chain).

In one embodiment, the cells are transformed with an expression cassette comprising from 5′ to 3′: at least one promoter operably linked to a first gene encoding a light chain of an antibody or antibody fragment and to a second gene encoding a heavy chain of the antibody or antibody fragment and wherein the expression cassette further comprises an internal ribosomal entry site (IRES) after the first gene encoding the light chain of the antibody or antibody fragment and before the second gene encoding the heavy chain of the antibody or antibody fragment and there is reduced antibody aggregation as compared to cells transformed with an expression cassette comprising from 5′ to 3′: at least one promoter operably linked to a first gene encoding a heavy chain of an antibody or antibody fragment and to a second gene encoding a light chain of the antibody or antibody fragment and wherein the expression cassette further comprises an internal ribosomal entry site (IRES) after the first gene encoding the light chain of the antibody or antibody fragment and before the second gene encoding the heavy chain of an antibody or antibody fragment. As described in further detail elsewhere herein the expression cassette may optionally comprise sequences providing or coding for WPRE at the 3′ end of the construct as represented diagrammatically in topmost construct of FIG. 1. Therefore, in one embodiment, the expression cassette comprises sequences providing or coding for WPRE after the second gene encoding the heavy chain of an antibody or antibody fragment. Note this applies to all relevant constructs and methods of the invention.

In a further embodiment, the cells are transformed with an expression cassette comprising from 5′ to 3′: a first promoter operably linked to a first gene encoding a light chain of an antibody or antibody fragment and a second promoter operably linked to a second gene encoding a heavy chain of the antibody or antibody fragment and there is reduced antibody or antibody fragment aggregation as compared to cells transformed with an expression cassette comprising from 5′ to 3′: a first promoter operably linked to a first gene encoding a heavy chain of an antibody or antibody fragment and a second promoter operably linked to a second gene encoding a light chain of the antibody or antibody fragment.

For example, there is an at least 10%, at least 20%, at least 30%, at least 40%, at least 50%, at least 60%, at least 70%, at least 80%, at least 90% reduction in antibody or antibody fragment aggregation.

The inventors also surprisingly observed increased aggregation of constructs containing furin and 2A (such as the 2A peptide derived from the foot and mouth disease virus, F2A) to allow self-cleavage. Thus, the expression cassettes preferably do not comprise self-cleavage peptides, in particular do not comprise furin/2A.

The inventors have also observed that the positioning of the gene encoding the light chain of an antibody or an antibody fragment relative to the gene encoding heavy chain of the antibody or the antibody fragment impacts the antibody titer. Indeed, when the gene encoding the light chain is the first gene in the expression cassette followed by the gene encoding the heavy chain there is increased titer of the expressed antibody or antibody fragment as compared to the reverse orientation (e.g. when the gene encoding the heavy chain is the first gene in the expression cassette followed by the gene encoding the light chain).

In a further aspect, the invention provides a method of increasing antibody or antibody fragment titer, wherein the method comprises:
(i) transforming cells with an expression cassette comprising from 5′ to 3′: at least one promoter operably linked to a first gene encoding a light chain of an antibody or antibody fragment and to a second gene encoding a heavy chain of the antibody or antibody fragment and wherein the expression cassette further comprises either an internal ribosomal entry site (IRES) or self (e.g. furin-2A) cleavage site after the first gene encoding the light chain of the antibody or antibody fragment and before the second gene encoding the heavy chain of the antibody or antibody fragment; or with an expression cassette comprising from 5′ to 3′: a first promoter operably linked to a first gene encoding a light chain of an antibody or antibody fragment and a second promoter operably linked to a second gene encoding a heavy chain of the antibody or antibody fragment; and
(ii) maintaining the transformed cells under conditions suitable for antibody or antibody fragment production.

Although this observation applies to self-cleavage sites, in particular furin-2A containing constructs, it is preferred that they are not included due to aggregation and immunogenicity issues as discussed herein.

In one embodiment, the cells are transformed with an expression cassette comprising from 5′ to 3′: at least one promoter operably linked to a first gene encoding a light chain of an antibody or antibody fragment and to a second gene encoding a heavy chain of the antibody or antibody fragment and wherein the expression cassette further comprises an internal ribosomal entry site (IRES) after the first gene encoding the light chain of the antibody or antibody fragment and before the second gene encoding the heavy chain of the antibody or antibody fragment. Thus, there is increased antibody titer as compared to cells transformed with an expression cassette comprising from 5′ to 3′: at least one promoter operably linked to a first gene encoding a heavy chain of an antibody or antibody fragment and to a second gene encoding a light chain of the antibody or antibody fragment and wherein the expression cassette further comprises an internal ribosomal entry site (IRES) after the first gene encoding the light chain of the antibody or antibody fragment and before the second gene encoding the heavy chain of the antibody or antibody fragment. As described in further detail elsewhere herein the expression cassette may optionally comprise sequences providing or coding for WPRE at the 3′end of the construct as represented diagrammatically in topmost construct of FIG. 1. Therefore, in one embodiment, the expression cassette comprises sequences providing or coding for WPRE after the second gene encoding the heavy chain of an antibody or antibody fragment.

In another (less preferred) embodiment, the cells are transformed with an expression cassette comprising from 5′ to 3′: at least one promoter operably linked to a first gene encoding a light chain of an antibody or antibody fragment and to a second gene encoding a heavy chain of the antibody or antibody fragment and wherein the expression cassette further comprises a self (e.g. furin-2A) cleavage site after the first gene encoding the light chain of the antibody or antibody fragment and before the second gene encoding the heavy chain of the antibody or antibody fragment and there is increased antibody titer as compared to cells transformed with an expression cassette comprising from 5′ to 3′: at least one promoter operably linked to a first gene encoding a heavy chain of an antibody or antibody fragment and to a second gene encoding a light chain of the antibody or antibody fragment and wherein the expression cassette further comprises a self (e.g. furin-2A) cleavage site after the first gene encoding the light chain of the antibody or antibody fragment and before the second gene encoding the heavy chain of the antibody or antibody fragment. As described in further detail elsewhere herein the expression cassette may optionally comprise sequences providing or coding for WPRE at the 3′ end of the construct as represented diagrammatically in topmost construct of FIG. 1. Therefore, in one embodiment, the expression cassette comprises sequences providing or coding for WPRE after the second gene encoding the heavy chain of an antibody or antibody fragment.

In a further embodiment, the cells are transformed with an expression cassette comprising from 5′ to 3′: a first promoter operably linked to a first gene encoding a light chain of an antibody or antibody fragment and a second promoter operably linked to a second gene encoding a heavy chain of the antibody or antibody fragment and there is increased antibody titer as compared to cells transformed with an expression cassette comprising from 5′ to 3′: first promoter operably linked to a first gene encoding the heavy chain of the antibody or antibody fragment and a second promoter operably linked to a second gene encoding the light chain of the antibody or antibody fragment.

For example, there is an at least 10%, at least 20%, at least 30%, at least 40%, at least 50%, at least 60%, at least 70%, at least 80%, at least 90% increase in antibody or antibody fragment titer.

Without wishing to be bound by theory, the inventors also consider that antibodies produced using expression cassettes, that do not utilize self-cleavage sites such as furin-2A, have reduced immunogenicity as compared to antibodies produced using expression cassettes that do utilize self-cleavage sites such as furin-2A. This is because the antibodies lack any remnants of the self-cleavage sites such as furin-2A peptide which are known to promote a neutralizing antibody response and/or cellular immunity to the antibody. As a consequence, it is considered that antibodies produced using expression cassettes, that do not utilize self-cleavage sites (such as furin-2A), result in a reduction of unwanted immunogenicity. The term “unwanted immunogenicity” as used herein is used to refer to an immune response by an animal (e.g. human) against the antibody (in particular directed to the peptide remnants of the self-cleavage peptides included in the expression cassettes) that leads to the production of neutralizing antibodies and/or cellular immunity, which diminishes the activity of the therapeutic antibody in vivo. In addition, said unwanted immunogenicity can be a cause of adverse events associated with antibody or antibody fragment therapy in vivo.

Thus, in an additional aspect, the invention provides a method of reducing unwanted antibody or antibody fragment immunogenicity, wherein the method comprises:
(i) transforming cells with an expression cassette comprising from 5′ to 3′: at least one promoter operably linked to a first gene encoding a light chain of an antibody or antibody fragment and to a second gene encoding a heavy chain of the antibody or antibody fragment and wherein the expression cassette further comprises an internal ribosomal entry site (IRES) after the first gene encoding the light chain of the antibody or antibody fragment and before the second gene encoding the heavy chain of the antibody or antibody fragment: or with an expression cassette comprising from 5′ to 3′: a first promoter operably linked to a first gene encoding a light chain of an antibody or antibody fragment and a second promoter operably linked to a second gene encoding a heavy chain of the antibody or antibody fragment; and
(ii) maintaining the transformed cells under conditions suitable for antibody or antibody fragment production.

Another aspect of the invention provides a method of reducing adverse events associated with antibody or antibody fragment therapy, wherein the method comprises:
(i) transforming cells with an expression cassette comprising from 5′ to 3′: at least one promoter operably linked to a first gene encoding a light chain of an antibody or antibody fragment and to a second gene encoding a heavy chain of the antibody or antibody fragment and wherein the expression cassette further comprises an internal ribosomal entry site (IRES) after the first gene encoding the light chain of the antibody or antibody fragment and before the second gene encoding the heavy chain of the antibody or antibody fragment: or with an expression cassette comprising from 5′ to 3′: a first promoter operably linked to a first gene encoding a light chain of an antibody or antibody fragment and a second promoter operably linked to a second gene encoding a heavy chain of the antibody or antibody fragment; and
(ii) maintaining the transformed cells under conditions suitable for antibody or antibody fragment production.

In one embodiment according to these aspects, the cells are transformed or transduced with an expression cassette comprising from 5′ to 3′: at least one promoter operably linked to a first gene encoding a light chain of an antibody or antibody fragment and to a second gene encoding a heavy chain of the antibody or antibody fragment and wherein the expression cassette further comprises an internal ribosomal entry site (IRES) after the first gene encoding the light chain of the antibody or antibody fragment and before the second gene encoding the heavy chain of the antibody or antibody fragment and there is reduced unwanted immunogenicity as compared to cells transformed with an expression cassette comprising from 5′ to 3′: at least one promoter operably linked to a first gene encoding a light chain of an antibody or antibody fragment and to a second gene encoding a heavy chain of the antibody or antibody fragment and wherein the expression cassette further comprises a furin-2A cleavage site after the first gene encoding the light chain of the antibody or antibody fragment and before the second gene encoding the heavy chain of the antibody or antibody fragment.

For example, there is an at least 10%, at least 20%, at least 30%, at least 40%, at least 50%, at least 60%, at least 70%, at least 80%, at least 90% reduction in antibody or antibody fragment immunogenicity and/or a corresponding reduction in adverse events associated with the antibody or antibody fragment therapy.

In the context of a comparator, the skilled person would understand that the same cells and the same conditions would be employed.

In a further embodiment, the antibody or antibody fragment is free of self-cleavage elements. In another embodiment, the antibody or antibody fragment is free of furin-2A peptides or fragments thereof.

In another aspect, the invention provides an antibody or antibody fragment obtained by the methods according to the invention. The antibody or antibody fragments obtained by the methods according to the invention may be used to reduce unwanted immunogenicity (which may include inflammation) in a subject as compared to antibodies or antibody fragments produced by methods which use expression cassettes comprising self-cleavage sites in between the genes encoding the heavy and light chains of an antibody or antibody fragment. In a further embodiment, the antibody or antibody fragment produced by the methods of the invention may also reduce any toxicity associated with antibodies produced by methods which use expression cassettes comprising self-cleavage sites in between the genes encoding the heavy and light chains of an antibody or antibody fragment.

The invention also provides corresponding expression cassettes that may be employed in such methods. Thus, there is provided an expression cassette comprising from 5′ to 3′: at least one promoter operably linked to a first gene encoding a light chain of an antibody or antibody fragment and to a second gene encoding a heavy chain of the antibody or antibody fragment; wherein the expression cassette further comprises an IRES after the first gene encoding the light chain of the antibody or antibody fragment and before the second gene encoding the heavy chain of the antibody or antibody fragment. Introduction of the expression cassette into cells produces a higher antibody titer as compared to an antibody titer produced when an expression cassette, comprising from 5′ to 3′: the at least one promoter operably linked to the first gene encoding the heavy chain of the antibody or antibody fragment and the second gene encoding the light chain of the antibody or antibody fragment and the IRES after the first gene encoding the light chain of the antibody or antibody fragment and before the second gene encoding the heavy chain of the antibody or antibody fragment, is introduced into identical cells. As described in further detail elsewhere herein the expression cassette may optionally comprise sequences providing or coding for WPRE at the 3′ end of the construct as represented diagrammatically in topmost construct of FIG. 1. Therefore, in one embodiment, the expression cassette comprises sequences providing or coding for WPRE after the second gene encoding the heavy chain of an antibody or antibody fragment. Similarly, the invention provides an expression cassette comprising from 5′ to 3′: a first promoter operably linked to a first gene encoding a light chain of an antibody or antibody fragment and a second promoter operably linked to a second gene encoding a heavy chain of the antibody or antibody fragment. Introduction of the expression cassette into cells produces a higher antibody titer as compared to an antibody titer produced when an expression cassette, comprising from 5′ to 3′: the first promoter operably linked to the first gene encoding the light chain of the antibody or antibody fragment and the second promoter operably linked to the second gene encoding the heavy chain of the antibody or antibody fragment, is introduced into identical cells.

These expression cassettes preferably do not include a self-cleavage site such as furin/2A cleavage site as such a construct leads to higher aggregation (i.e. lower quality) of the expressed antibodies and also increased immunogenicity.

The invention also relates to a vector that transduces or transfects cells of the blood brain barrier (BBB) or the CNS comprising an expression cassette of the invention as described herein. Such vectors are useful in methods of antibody or antibody fragment production, comprising: transforming cells with the vector and maintaining the transformed cells under conditions suitable for antibody or antibody fragment production.

The expression cassettes described herein were designed with delivery via viral vectors in mind. In such vectors, there are limits to the amount of genetic material that can be packaged. Thus, the invention provides a viral vector, in particular a neurotropic viral vector or a viral vector that can transduce or transfect cells of the BBB or the CNS comprising an expression cassette of the invention. The viral vector may comprise an engineered AAV2 vector, preferably AAV-BR1 or an engineered AAV9 vector, such as AAV-S, AAV-F, AAV-PHP.eB or AAV9-PHP-V1 or an engineered AAV1 vector, such as AAV1RX, AAV1R6 or AAV1R7. The viral vector may comprise an expression cassette comprising from 5′ to 3′: at least one promoter operably linked to a first gene encoding a light chain of an antibody or antibody fragment and to a second gene encoding a heavy chain of the antibody or antibody fragment and further comprises an IRES after the first gene encoding the light chain of the antibody or antibody fragment and before the second gene encoding the heavy chain of the antibody or antibody fragment. The viral vector may comprise an expression cassette comprising from 5′ to 3′: a first promoter operably linked to a first gene encoding a light chain of an antibody or antibody fragment and a second promoter operably linked to a second gene encoding a heavy chain of the antibody or antibody fragment.

These vectors and expression cassettes may be employed in any of the methods in accordance with the invention.

### BRIEF DESCRIPTION OF THE FIGURES

FIG. 1. Representation of the different constructs investigated to produce pAAV-derived antibodies, linked-derivate and other proteins. Curved arrows indicate alternation between light and heavy chain positions in the expression cassette. SP: Secretion Peptide, IRES: Internal Ribosome Entry Site, FSG2A: Furin/2A self-cleavable peptide, WPRE: Woodchuck Hepatitis Virus Posttranscriptional Regulatory Element, pA: Poly A. Heavy chain or Light chain can be any antibody sequences. Promoter can be preferably CMV (human cytomegalovirus) or other promoters such as, but not limited to EF1A (Human Eukaryotic translation elongation factor 1 alpha 1), CAG (CMV early enhancer fused to chicken β-actin promoter), CBh (CMV early enhancer fused to modified chicken β-actin promoter), SV40 (Simian virus 40 enhancer/early promoter), GFAP (Human glial fibrillary acidic protein promoter) *: 3′ regulatory elements not included.

FIG. 2A. CHO cell supernatant titers of MAB1 IgG and scFv-Fc following pAAV transfection. BLI Octet system, protein A-coated biosensors. Arrows indicate a clear decrease in expression when heavy chain gene is positioned prior the light chain in the construct (HC/LC) compared to their respective LC/HC counterparts. Clone constructs are according to the options of FIG. 1 as detailed in the Figure.

FIG. 2B. CHO cell supernatant titers of MAB1 Fab following pAAV transfection. BLI Octet system, anti-His-coated biosensors. Arrows indicate again a clear down expression when heavy chain gene is positioned prior the light chain in the construct (HC/LC) compared to their respective LC/HC counterparts.

FIG. 3. SDS-PAGE separation of purified MAB1 IgGs with Coomassie blue staining. Samples were reduced in the presence of 5 mM dithiothreitol when indicated (DTT). Arrows indicate a shift to a larger molecular weight.

FIG. 4. SDS-PAGE separation of purified MAB1 scFv-Fc with Coomassie blue staining. Samples were reduced in the presence of 5 mM dithiothreitol when indicated (DTT).

FIG. 5. MAB1 IgG binding to TP-62 peptide. Octet-Kinetics starting at 100 nM binder protein candidate followed by 2-fold serial dilution. Upper panel covers 2 promoter MAB1 IgG with different promoters and a furin HC/LC IgG construct. Lower panel covers furin HC/LC and LC/HC MAB1 IgG construct, IRES LC/HC and HC/LC IgG construct as well as scFv-Fc IgG constructs. Individual construct details are found in the figure. Each panel starts with the biosensor loading using 500 nM TP-62 peptide.

FIG. 6. Size exclusion chromatography analysis of relevant purified pAAV-derived IgG using Superdex 200 Increase 10/300GL column. Separation was performed at PBS buffer at 4° C. Individual construct details are found in the figure.

FIG. 7. Size exclusion chromatogram superposition of relevant purified pAAV-derived IgG. Same conditions as described in FIG. 6.

FIG. 8. Analysis of the C-terminal parts of furin MAB2 IgG constructs confirming the furin/F2A peptide left over and thereby increase of mass of the first antibody chain on the construct. In gel trypsin digest followed by LC/MS.

FIG. 9. Purified MAB1 IgG and scFv-Fc per culture mL coming from AAV2, 8, 9 and 10 CHO transductions and compared to transfections. Purified protein quantities per mL were quantified by OD 280 nm using their corresponding coefficient of extinction. Individual construct details are found in the figure.

FIG. 10. SDS-PAGE separation of purified MAB1 IgGs with Coomassie blue staining. Samples were reduced in the presence of 5 mM dithiothreitol when indicated (DTT).

FIG. 11. IgG titer comparison following transduction of CHO, differentiated neurons and BBB cells with AAV2, 8, 9 and 10-vectorized antibody constructs. Left panel, CHO transduction; middle panel differentiated human neuroblastoma cell line transduction; right panel differentiated blood brain barrier (BBB) cell transduction. Individual construct details are found in the figure.

FIG. 12. IgG titer comparison following transduction of rat brain primary cells with AAV2, 8, 9 and 10-vectorized MAB1 IgG and scFv-Fc constructs. Individual construct details are found in the figure.

FIG. 13. Detection of MAB1 (a human IgG1 isotype) secreted by hCMEC/D3 cells into the apical supernatants of an Organ® Plate® 3D BBB model 3 days after transduction by different WT AAV serotypes. Expression of MAB1 was driven by CMV promoter. IRES element was used between LC and HC to achieve bicistronic antibody production. In summary, the constructs tested had the following arrangement: 5′-CMV promoter-light chain encoding gene with a sequence encoding a secretion peptide-IRES-heavy chain encoding gene with a sequence encoding a secretion peptide-3′. Antibody presence measured by ELISA binding to hTDP-43 full length (FL) protein is expressed as O.D. (Optical Density). No binding was observed for the control AAV2-eGFP transduction. As expected, no antibody binding was detected for the 2 controls, mlgG MAB1 and hlgG MAB2, as an anti-human secondary antibody was used for detection and MAB2 cannot bind hTDP-43 FL protein.

FIG. 14. Detection of MAB1 (a human IgG1 isotype) secreted by hCMEC/D3 cells in both apical and basolateral compartments of an Organ® Plate® 3D BBB model 3 days after transduction by an AAV2 construct. Bicistronic expression of MAB1 driven by CMV promoter was achieved using IRES element between LC and HC. In summary, the constructs tested had the following arrangement: 5′-CMV promoter-light chain encoding gene with a sequence encoding a secretion peptide-IRES-heavy chain encoding gene with a sequence encoding a secretion peptide-3′. Antibody presence measured by ELISA binding to TDP-43 full length protein. No binding was observed for the control AAV2-eGFP transduction. Data expressed as O.D.

FIG. 15. Detection of MAB1 (a human IgG1 isotype) secreted by hCMEC/D3 cells into the supernatant of a 24-well culture plate 3 days after transduction by an AAV2 construct at different MOI (Multiplicity of infection). Bicistronic expression of MAB1 driven by CMV promoter was achieved using IRES element between LC and HC. In summary, the constructs tested had the following arrangement: 5′-CMV promoter-light chain encoding gene with a sequence encoding a secretion peptide-IRES-heavy chain encoding gene with a sequence encoding a secretion peptide-3′. Antibody presence measured by ELISA binding to TDP-43 full length protein. Data are expressed in concentration calculated from a standard curve.

FIG. 16. Detection of MAB1 (a human IgG1 isotype) secreted by hCMEC/D3 cells into both apical and basolateral compartments of a Transwell BBB model 3 days after transduction by an AAV2 construct. Bicistronic expression of MAB1 driven by CMV promoter was achieved using IRES element between LC and HC. In summary, the constructs tested had the following arrangement: 5′-CMV promoter-light chain encoding gene with a sequence encoding a secretion peptide-IRES-heavy chain encoding gene with a sequence encoding a secretion peptide-3′. Antibody presence measured by ELISA binding to hTDP-43 full length protein. No binding was observed for the control AAV2-eGFP transduction (data not shown). Data are expressed in relative percentage of antibody present.

FIG. 17. Detection of MAB1 hIgG1 and scFv-Fc in cell supernatant 6 days after transduction by AAV2, AAV-BR1 and AAVrh10 constructs at a MOI of 100′000 in (A) b.End3 and b.End5 mouse brain endothelioma cell lines and (B) hCMEC/D3 cells. Bicistronic expression of MAB1 driven by CMV promoter was achieved using IRES element between LC and HC. In summary, the constructs tested had the following arrangement from 5′ to 3′:CMV promoter-secretion peptide-light chain-IRES—secretion peptide-heavy chain—WPRE-polyA. Antibody presence measured by HTRF (Homogeneous Time Resolved Fluorescence) using an anti-hFc kit (PerkinElmer, Cisbio). Data expressed in concentration interpolated from a standard curve and generated from 3 to 6 biological replicates within a single experiment with mean±SD plotted.

FIG. 18. Detection of MAB1 hIgG1 or mIgG2a secreted by primary human brain microvasculature endothelial cells from a commercially available 3D human in vitro BBB model (Neuromics). Detection performed in both apical and basolateral compartments 7 days after transduction by AAV2 and AAV-BR1 vectors at a MOI of 100′000. Bicistronic expression of MAB1 driven by CMV or CBh promoter was achieved using IRES element between LC and HC. In summary, the constructs tested had the following arrangement from 5′ to 3′:CMV or CBh promoter secretion peptide-light chain-IRES—secretion peptide-heavy chain—WPRE-polyA. Antibody presence measured by HTRF using an anti-hFc kit (PerkinElmer, Cisbio). Data expressed in (A) concentration or (B) quantity interpolated from a standard curve in respective apical and basolateral compartments. To allow direct comparison between evaluated conditions, 2 to 3 biological replicates were performed within a single experiment with mean±SD plotted.

The invention is further described in the following Examples. The Examples serve only to illustrate the invention and are not intended to limit the scope of the invention in any way.

### EXAMPLES

### Example 1: Vectorized Antibody Constructs

1. Selecting optimal clones for vectorization

1.1. Introduction

One of the main challenges with using the single strand DNA (ssDNA) vectorization in AAV capsids is capacity. The expression cassette size contains a) the promoter, b) the open reading frame (encoding an antibody or antibody fragment, typically including a heavy and a light chain) and c) downstream regulatory elements. Once the DNA is converted to single strand and vectorized in AAV capsids, the recommended size is not larger than 4.7 kb including the 5′ITR and 3′ITR. Larger sized constructs will hardly enter the AAV viral capsid complex and thereby lower the production yield. Self-complementary constructs have an even smaller capacity within the 5′ITR and 3′ITR, i.e. not larger than 2.3 kb.

Commonly used antibody plasmid AAV (pAAV) designs are made with antibody fragments that are smaller in size and fit the expression cassette driven by a single promoter such as a) antibody single-chain variable fragment (scFv)[44, 55], b) scFv fused to IgG Fc domain (scFv-Fc)[56, 57] or c) single domain antibodies, such as sharks and/or Camelidae antibodies[58-62].

In the case of full antibodies, it is challenging to fit both light and heavy chain genes due to the aforementioned size-restrictions of the expression cassette. To date, most of academic and industrial researchers rely on pAAV constructs consisting of an open reading frame translated in a self-processing protein fusion. In most cases, the respective coding order consists of 1) an antibody heavy chain, 2) a furin protease recognition site also known as PACE (Paired basic Amino acid Cleaving Enzyme)[63], 3) a self-cleaving 2A peptide facilitating the furin cleavage[47-49] and expected to promoting a cleaner process, namely E2A, F2A, P2A or T2A, and 4) an antibody light chain. Researchers have a significant preference for this construct due to obtained high expression titers and equimolarity in antibody chain expression. However, and in most cases, the proteins are not properly maturated with remnants of the furin and 2A fusion peptides included in the expressed protein thereby potentially leveraging an unwanted immunogenicity of the expressed proteins[45]. Different 2A peptides have different efficiencies of self-cleaving, T2A and P2A being the most and F2A the least efficient[65]. Therefore, up to 50% of F2A-linked proteins can remain in the cell as a fusion protein, which might cause some unpredictable outcomes, including a gain of function[66]. 2A sites cause the ribosome to detach from polynucleotides approximately 60% of the time. Together with ribosome read-through of about 10% for P2A and T2A, this results in reducing expression of the downstream peptide chain by about 70%[47]. Furthermore, it is important to note that 2A peptides are derived from viruses,—i.e. F2A is derived from foot-and-mouth disease virus 18; E2A is derived from equine rhinitis A virus; P2A is derived from porcine teschovirus-1 2A; T2A is derived from thosea asigna virus 2A. Hence, leftover 2A peptide residues on either the heavy chain or light chain could be considered as non-self by mammalian and human immune systems.

Less common pAAV construct alternatives to furin/2A fusions for antibody expression are using 1) two promoters, each driving the heavy and light antibody chains or 2) one promoter and positioning an internal ribosome entry site (IRES, DNA sequence derived from encephalomyocarditis virus) between the two antibody gene chains. The first increases restrictions in the pAAV design due to the commonly large size of promoters, thereby necessitating the use of small promoters to control heavy and light chain antibody genes and to remove 3′ITR regulatory elements that can greatly increase protein titer. The second, IRES construct, is considered to date a poor protein expresser[56, 67-69] and thereby disregarded for many pAAV constructs (where IRES construct expresses much lower titers than 2A or furin 2A constructs).

1.2. Selection Approach

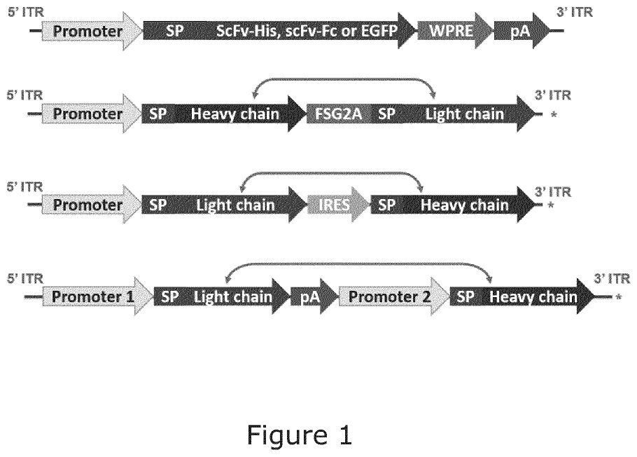

We screened more than 55 pAAV constructs that were produced and investigated for expression depending on open reading frame component positions, promoter strength, secretion peptides and fusion peptide. The different type of constructs are illustrated in FIG. 1. To our knowledge full IgG expression using AAV vectors reported in the literature have a furin/2A construct that has the heavy chain positioned prior the light chain (see FIG. 1, 2nd construct). Our approach investigated also other light and heavy chain positions for this type of construct and different promoter strengths for the bicistronic constructs (FIG. 1, 4th construct). Furthermore, we challenged the reported low expression for IRES bicistronic constructs by alternating the chain positions, using different secretion peptides and customizing intra- and down-stream regulatory elements (FIG. 1, 3^{rd} construct). Finally, we tested two full antibody clones and corresponding antibody fragments (FIG. 1, 1st construct) such as scFv-Fc, Fab and scFv, namely MAB1 (an anti-TDP-43 antibody) and MAB2 (an anti-ErbB2 antibody). In addition, we included clones expressing the enhanced green fluorescent protein (eGFP).

In our studies, we defined the selection criteria to identify the best pAAV constructs as follows: 1) high protein titer, 2) low aggregate level, 3) accurate maturation and folding and 4) binding to target.

1.3. Chinese Hamster Ovary (CHO) Cell Transfection and Supernatant Titer

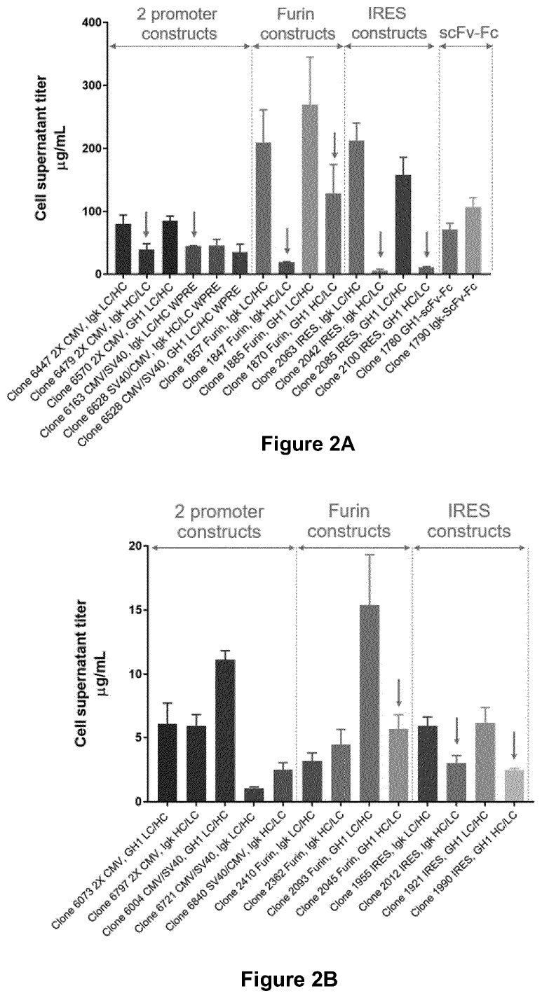

In brief, CHO cells (ExpiCHO-S™ (ThermoFisher, cat: A29127)) were transfected following manufacturer's recommendations with 1 μg/mL plasmid per construct in triplicate. Cells were grown in 24 deep well microplates with optimized synthetic medium at 37° C. for 24h, followed by a temperature shift to 32° C. and then grown for 11 days in a final volume of about 3.5 mL medium. Titers were then estimated in each supernatant triplicate using Bio-Layer Interferometry (BLI) with the Octet system and biosensor tips coated with either a) protein A in the case full length antibodies and scFv-Fcs or b) a monoclonal antibody binding His-tagged proteins, here Fabs, scFvs and EGFP. For all measures, a standard curve was performed using the corresponding and previously purified protein. The results are shown for antibodies and scFv-Fcs in FIG. 2A and FIG. 2B for Fabs.

Observations. In the case of MAB1 IgG, each 2 promoter construct produced significant titers ranging between approximately 30 to 100 μg/mL. Surprisingly, approximately 2-to-10-fold higher titers ranging from 150 to 350 μg/mL were obtained with the furin and IRES constructs, but remarkably only when the light chain gene was positioned prior the heavy in the construct (LC/HC) as compared to the 2 promoter IgG constructs. The high protein levels in the IRES IgG construct LC/HC were comparable to the furin IgG constructs. This was unexpected and, to our knowledge, reported for the first time as the state of the art mentions the opposite[56, 67-69]. A similar observation was made for the MAB1 Fab constructs in FIG. 2B, indicating a similar titer to the furin and 2 promoter constructs. In addition, the position of the light chain prior the heavy chain (LC/HC) in the IRES Fab constructs have significantly higher expression titers than their HC/LC construct counterparts. The collected data indicates that in IgG or Fab construct using furin and IRES, the light chain positioned before the heavy chain increases the titer. The titer is significantly higher than the HC/LC counterpart. Finally, the expression seems lowered when the medium strength promoter SV40 is included as one of the two promoters in the two promoter IgG construct.

1.4. Protein Purification and Analysis on SDS-PAGE

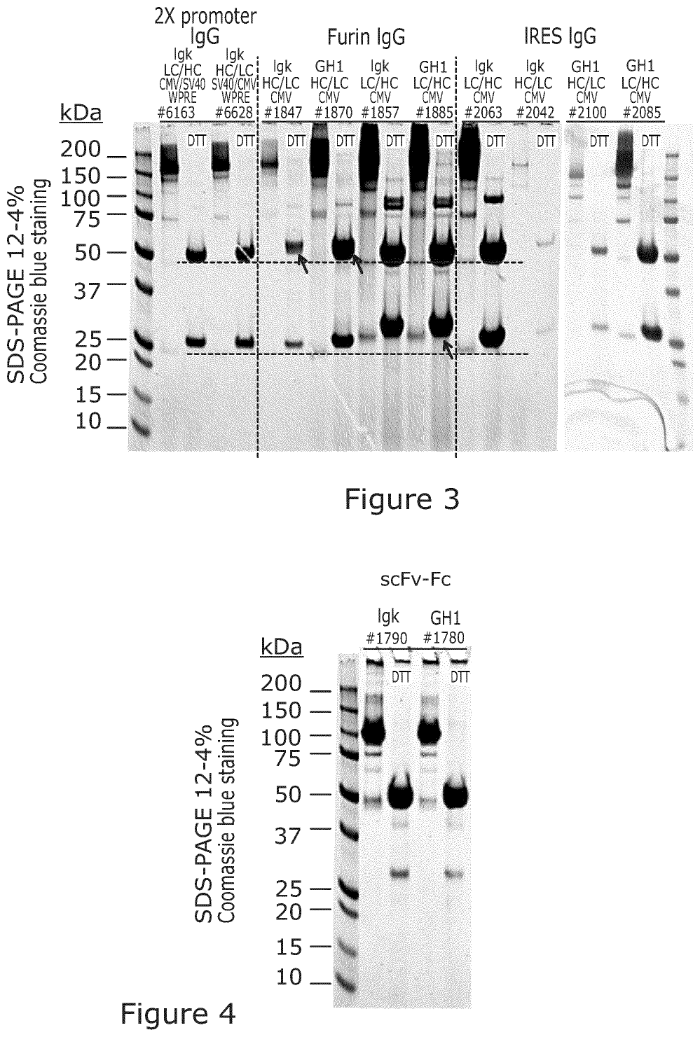

Following this experiment, cells were harvested, and supernatant triplicates were separated and combined per clone. In the case of the full-length antibody and scFv-Fc clones, proteins were captured using a commercially available protein A resin and eluted them with 100 mM glycine pH 2.8 buffer supplemented by 100 mM NaCl. Proteins were then quantified by spectrophotometry OD 280 nm using their corresponding coefficient of extinction. In the case of Fabs and scFvs constructs, we employed a commercially available affinity column for His-tagged proteins. Proteins were then separated by SDS-PAGE 12-4% and stained with Coomassie blue. Independently from the protein concentration, 13 μL per sample were loaded per gel well to: a) have a second estimation of the purified titers and b) see any possible degradation products. In addition, verification of interchain disulfide bond formation and proper complex at 150 kDa was performed in the absence of dithiothreitol (DTT) and the release of light chain and heavy chain in the presence of dithiothreitol. The resulting gel for purified MAB1 IgG pAAV clones is presented in FIG. 3.

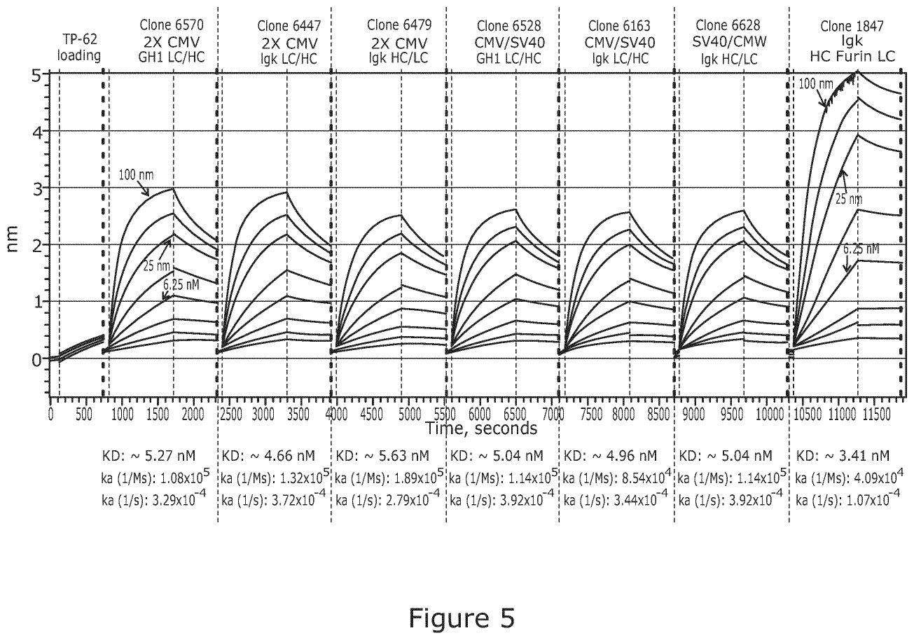

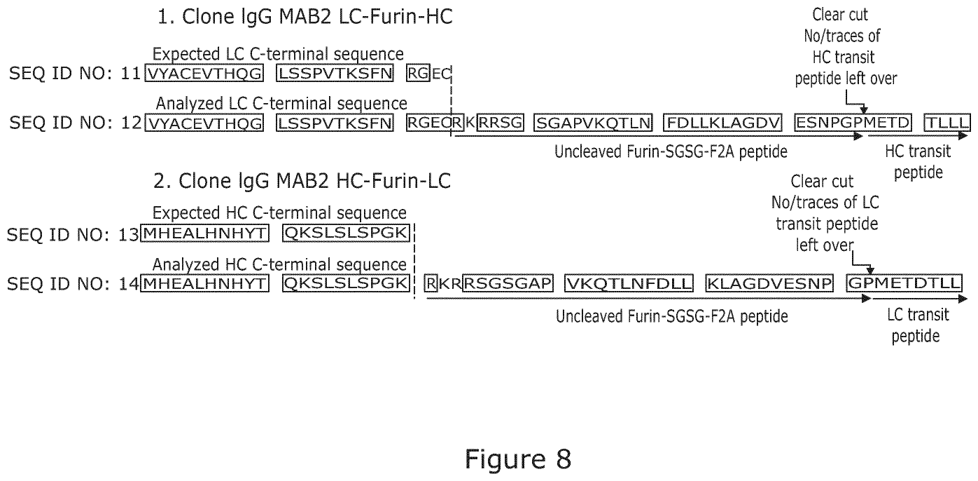

Observations. All samples had a proper disulfide bond configuration and antibodies migrated at the corresponding molecular weight of 150 kDa. When reduced, both constructs, 2 promoter and IRES IgGs, were separated at the expected light and heavy chain molecular weight of respectively ˜25 and ˜50 kDa. However, it was noticed that the first chain of the furin/2A IgG construct was larger in molecular weight, i.e. the heavy chain in the HC/LC construct configuration and light chain in the LC/HC configuration. The size of each furin/2A IgG chain construct was further investigated by LC/MS for peptide accuracy. This confirmed an addition of the full or some part of furin/2A peptide to the C-terminal of either the light for the LC/HC construct or heavy chain for the HC/LC construct (see FIGS. 3, 7 and 8). In contrast, the second antibody construct chain was processed properly, and no peptide remnants were observed by LC/MS at the N-terminus. Overall, all clones indicated low levels of degradation under the applied cell culture conditions. The 2 promoter and IRES proteins seem to have the most accurate maturation quality. Finally, the same observations were made with clone MAB2, a different IgG antibody. Again, the furin/2A constructs had larger molecular weights for the first construct chain and addition of the furin/2A peptide was confirmed by LC/MS, whereas the 2 promoter and IRES IgG had the most accurate maturation. Purified MAB1 scFv-Fc samples were also separated in the same SDS-PAGE conditions. The result is presented in FIG. 4.

Observations. As per the purified MAB1 samples, the scFv-Fc has a proper interchain disulfide bond formation and the protein migrates at its expected molecular weight of ˜100 kDa. A single chain is released when the sample is reduced with 5 mM DTT and migrates at the expected molecular weight of 50 kDa. Overall, all clones indicated low levels of degradation in the used cell culture conditions.

### 1.5. Analysis of Binding Affinity of Transfection-Derived Proteins by BLI

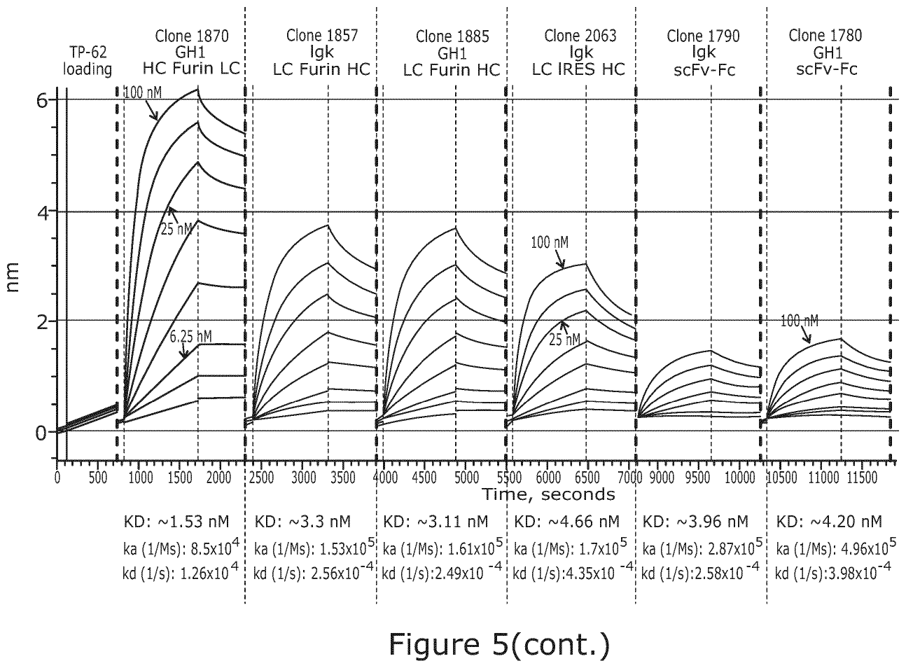

To demonstrate that the pAAV system-generated proteins were functional, we measured their binding affinity to a TAR DNA-binding protein 43 (TDP-43)C-terminal peptide by BLI using the Octet system. In brief, streptavidin biosensor tips were coated with 500 nM TDP-43 C-terminal peptide called TP-62. Measures were then performed using a reaction buffer consisting of PBS supplemented by 0.1% bovine serum albumin and 0.02% Tween. The reactions were performed at 30° C. The samples were then analyzed with two-fold serial dilutions starting at 100 nM concentration. Protein molarities were calculated using their corresponding coefficient of extinction at OD 280 nm. Sample association to TP-62 peptide was allowed for 900 seconds in reaction buffer, followed by a 600 second dissociation in the same buffer. Biosensors were regenerated in 10 mM glycine pH 2.0 and neutralized in reaction buffer prior each sample measure. The collected data is presented in FIG. 5 for relevant purified samples.

Observations. Overall, all MAB1 clones are functional and bind the TDP-43 C-terminal peptide with a comparable K_{D} that ranges from ˜1-6 nM. The 2 promoter and IRES constructs had a similar Rmax ranging between ˜2.5 and 3 nm. Importantly, the 2 promoter constructs K_{D} affinity did not vary as a function of the promoter or secretion peptide, respectively (CMV/CMV, CMV/SV40 or SV40/CMV promoter constructs and either Igk or GH1 secretion peptide). In contrast, furin IgG HC/LC constructs produced proteins with an approximately doubled Rmax than the 2 promoter and IRES constructs, respectively ˜5-6 nm versus ˜2.5-3 nm. In addition, the furin IgG LC/HC construct has less pronounced but also larger Rmax that its 2 promoter and IRES counterparts, respectively ˜3.5 nm versus ˜2.5-3 nm. According to the BLI system, the data indicates larger molecular weight proteins for the furin/2A constructs at equimolar concentrations compared to its 2 promoter and IRES counterparts, for instance the furin IgG HC/LC protein in the assay conditions. This observation was confirmed in the next analysis step using a size exclusion chromatography separation (see 1.6). Finally, the data demonstrates that functional IgG and scFv proteins can be obtained with the pAAV expression system. Further Octet analysis is described below (see 1.6).

1.6. Protein Analysis by Size Exclusion Chromatography and LC/MS

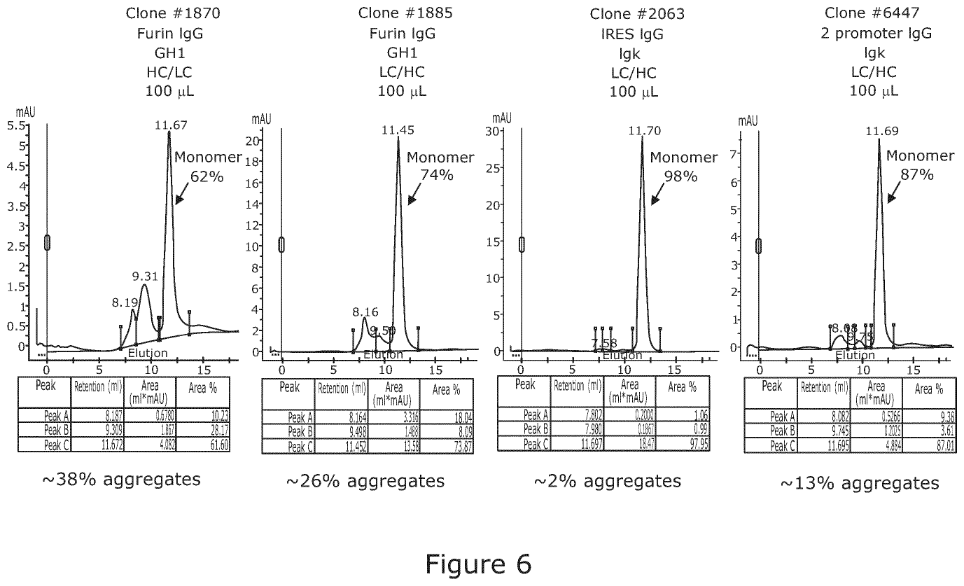

Purified proteins where then separated by size exclusion chromatography to monitor the presence of degradation, aggregates and proper protein folding separated at the molecular weight of 150 kDa as a monomer. For this, we separated 100 μL purified sample on Superdex 200 Increase 10/300GL column from G&E Healthcare at 4° C. using PBS buffer. The resulting separations are presented in FIG. 6.

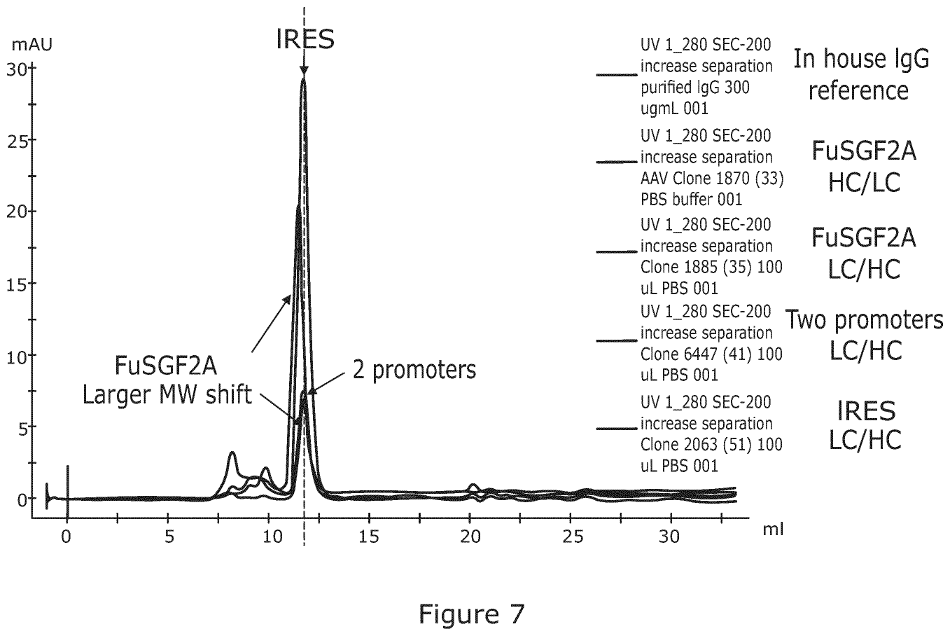

Observations. For all constructs, IgG monomers were separated as expected at an elution volume of about 11.7 mL. In contrast, the furin IgG constructs had large levels of aggregates; 26% for the LC/HC construct and 38% for the HC/LC construct. Furthermore, the monomeric peak of the furin construct is not aligned to the IRES and 2 promoter IgG constructs (see superposed FIG. 7) and has a slightly larger molecular weight in the separation eluting earlier. It is clearly the case for the Furin IgG LC/HC construct that elutes 0.25 mL earlier. The larger aggregate level in furin/2A constructs (FIGS. 6, 7 and 8) may reflect the reported unexpected outcome and toxicity of this type of protein[66]. These data confirm the above BLI measure indicating larger molecular weight furin/2A-derived IgG. To the contrary, the IRES IgG construct protein displayed very low 2% aggregate levels and a rich monomeric protein of 98%, thus demonstrating a higher quality. The 2 promoter IgG protein had as well a low aggregate level of 13% (although higher that the IRES construct). These results identified IgG IRES LC/HC and 2 promoters LC/HC as the constructs with better quality, expression yield, stability, and potentially less unwanted immunogenicity and/or toxicity.

2. Vectorization of MAB1 and MAB2 Lead Candidates

2.1. AAV Capsid Choices for Cell Transductions

As discussed above MAB1 and MAB2 best constructs providing the highest quality proteins were selected for vectorization, namely a) MAB1 IgG IRES LC/HC and 2 promoter IgG LC/HC constructs, b) MAB2 2 promoter IgG LC/HC and c) MAB1 scFv-Fc. A panel of different cells of interest were selected for transduced gene delivery, namely chinese ovarian hamster cells (CHO), Human neuroblastoma cell line differentiated in neurons and brain endothelial cell line (hCMEC/D3). For this, the above best pAAV constructs were vectorized in AAV2, AAV8, AAV9 and AAV10 capsids. The resulting ultra-purified capsids with low endotoxins were used to transduce cells.

2.2. CHO Cell Transductions, Protein Purification and Titer Comparison to Transfected Same Clones

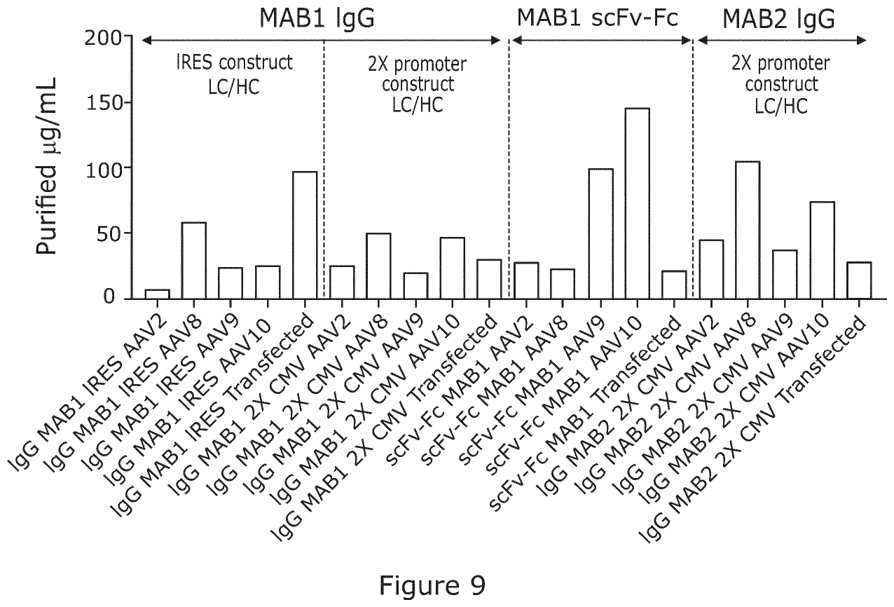

In brief, vectorized lead constructs were used to transfect CHO cell cultures at 100K gc (genome copy)/CHO cell in triplicates and then, grown in optimized synthetic medium as above in 24 deep well microplates 24h at 37° C., followed by a temperature shift to 32° C. and then growth for 11 days in a final volume of 3.5 mL per well. Cell growth was not affected by the AAV presence. After that, cells were harvested, and supernatant triplicates were separated and combined per clone. Full length antibody and scFv-Fc clones were captured as above using protein A resin and eluted them with 100 mM glycine pH 2.8 buffer supplemented by 100 mM NaCl. Proteins were then quantified by spectrophotometry OD 280 nm using their corresponding coefficient of extinction. The resulting titers are shown in FIG. 9.

Observation. All transduced lead gene constructs were expressed using each tested AAV capsid. The level of expression varies as a function of the capsid used for the vectorization but is similar overall to the same constructs transfected with the purified plasmids.

2.3. Rat Primary Brain Cell Transductions and Antibody Titer Using MAB1 Antigen ELISA

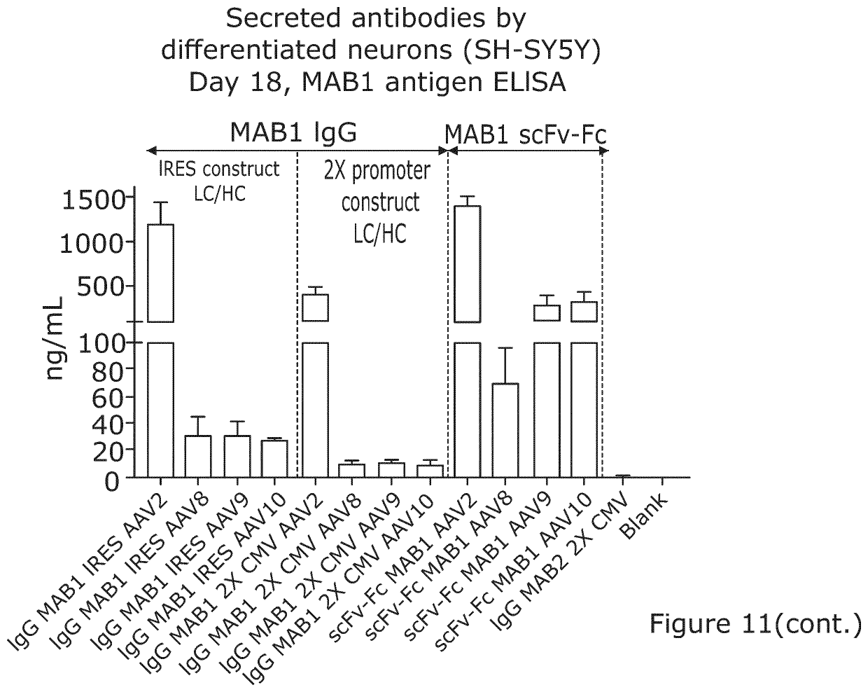

In brief, rat primary cells were obtained by dissection from rat pup brain and grown at 37° C. in 100 μL of neurobasal medium supplemented with B27™ (ThermoFisher, cat: 17504044) in 96 well microplates, containing 50K primary cells per well. Vectorized lead constructs were used to transduce rat primary brain cells at 100K gc/rat primary cell in triplicates and then, grown in neurobasal medium supplemented with B27 for 7 days at 37° C. Microscopic morphology evaluation indicated that cells were not affected by the AAV presence. Triplicate transduction cell supernatants were then collected to quantify antibody titers against hTDP-43 full length protein by ELISA. In brief, 96 well microplates were coated with 1 μg/ml human full length TDP-43 overnight in PBS buffer at 4° C. Plates were then washed 3 times with PBS supplemented with 0.05% Tween and then blocked for 1 hour at 37° C. with PBS, 0.05% Tween supplement with 1% bovine serum albumin. Collected antibody-containing supernatants were diluted, 20, 40 and 80 fold in blocking buffer and 50 μL samples were added to the microplate and incubated for 1 hour at 37° C. Plates were then washed as above and incubated with goat anti-human IgG Fc-HRP (abcam, #ab98624) diluted 1/10000 dilution in blocking buffer for 1 hour at 37° C. Plates were washed as above and wells were supplemented by 100 μL TMB substrate, incubated a few minutes at room temperature. After that, the HRP reactions were blocked with 50 μL of H₂SO₄ 0.16M. Finally, the resulting solutions were red at 450 nm with a microplate reader (BioTek). The corresponding antibody titers are shown in FIG. 12. The data indicates that MAB1 IgG and scFv-Fc clones vectorized in AAV2 have a significantly lower expression titer ranging between 5 and 50 ng/mL of cell supernatant. In contrast, other AAV8, 9 and 10-vectorized candidates have much larger IgG and scFv-Fc titers ranging between ˜500 and 2000 ng/mL of cell supernatant, irrespective of the expression-driven system (IRES or 2 promoters), except for MAB1 vectorized in AAV9, expressed under 2 CMV promoters with a titer of ˜180 ng/m L.

Observation. All transduced lead gene constructs were expressed using each tested AAV capsid. As above, the level of expression varies as a function of the capsid used for the vectorization but is similar overall to the same constructs transfected with the purified plasmids.

2.4. Analysis by SDS-PAGE to Verify Protein Maturation

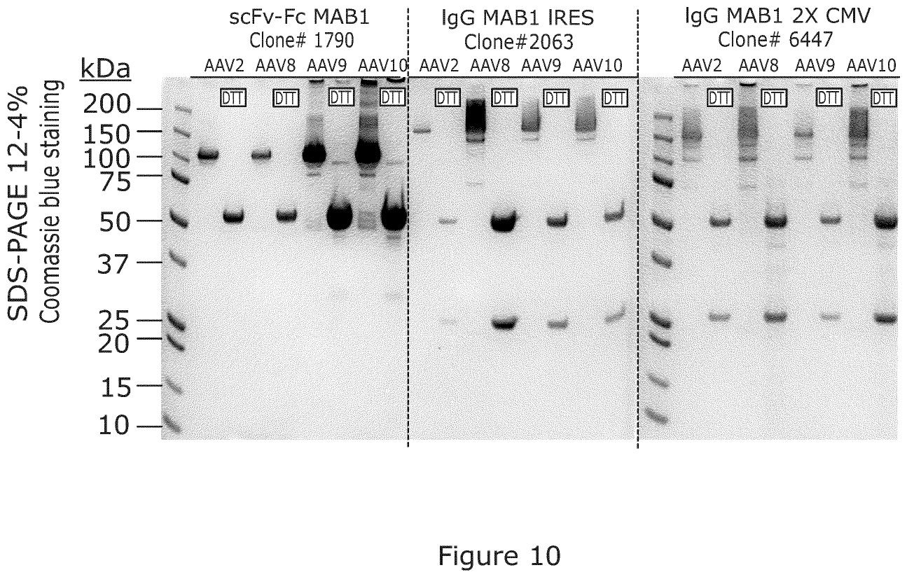

Purified lead proteins were separated by SDS-PAGE 12-4% and stained with Coomassie blue. As above, protein samples were loaded equally in volume (13 μL/sample) without harmonizing the loaded quantities in order to verify the measured quantities by OD 280 nm. The resulting separation is presented in FIG. 10 for the MAB1 pAAV clones.

Observation. All samples had a proper disulfide bond configuration and antibodies migrated at the corresponding molecular weight of 150 kDa as displayed in FIG. 10. When reduced, both constructs, 2 promoter and IRES IgGs, were separated at the expected light and heavy chain molecular weight of respectively ˜25 and ˜50 kDa. The scFv-Fc have a proper interchain disulfide bond formation and the protein migrates at its expected molecular weight of ˜100 kDa. A single chain is released when the sample is reduced with 5 mM DTT and migrates at the expected molecular weight of 50 kDa. Overall and as expected from the previous screening experiments, all clones indicated low levels of degradation in the adopted cell culture conditions.

2.5. Analysis of Binding Affinity of Transfection-, Transduction-Derived Proteins by BLI

To demonstrate that the generated proteins using the pAAV system were functional, we measured their binding affinity to a TAR DNA-binding protein 43 (TDP-43)C-terminal peptide by BLI using the Octet system in the same conditions as described above. In brief, streptavidin biosensor tips were coated with 500 nM TDP-43 C-terminal peptide called TP-62. Measured were then performed using a reaction buffer consisting of PBS supplemented by 0.1% bovine serum albumin and 0.02% Tween. The reactions were performed at 30° C. The samples were then analyzed with two-fold serial dilutions starting at 100 nM concentration. The collected data is presented in Table 1 below:

TABLE 1

| MAB1 antibody | K_{D} (nM) |
|---|---|
| IgG standard | 5.7 |
| IgG IRES pAAV transfected | 4.7 |
| IgG IRES pAAV vectorized | 5.7 |
| IgG 2 promoters pAAV transfected | 4.7 |
| IgG 2 promoters pAAV vectorized | 4.3 |
| scFv-Fc standard | 12.3 |
| scFv-Fc pAAV transfected | 4.0 |
| scFv-Fc pAAV vectorized | 21.25 |

Observation. The data indicate that the IgG clones either from IRES or 2 promoter pAAV constructs have comparable K_{D}s to the standard used (Table 1). Furthermore, the K_{D}s are comparable between the construct type either transfected or vectorized, ranging between ˜4 to 6 nM, thus demonstrating the vectorization generated high quality proteins with the expected binding affinity to the targeted TDP-43 antigen. The same can be said regarding the scFv-Fc proteins, with perhaps a slight variation.

2.6. Human Neuroblastoma Cell Line and BBB Cell Transductions and Protein Titers in Cell Supernatants

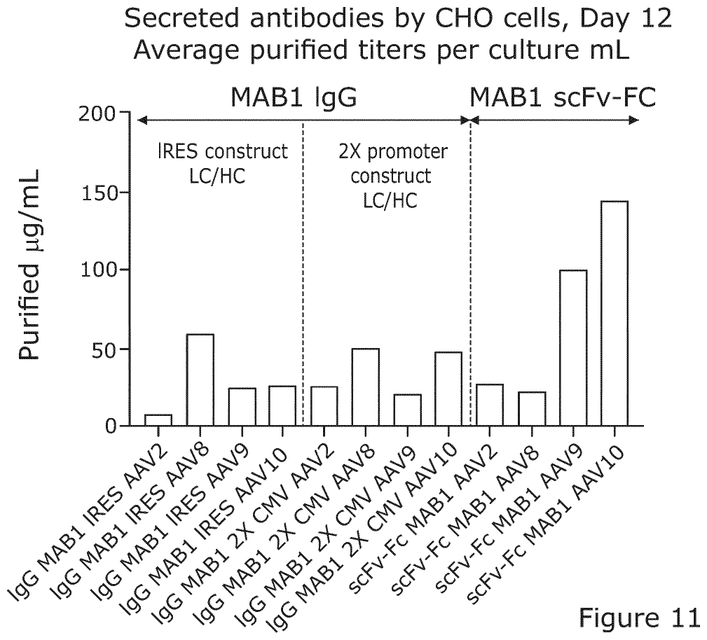

The next step was to verify whether other cell types than CHO could be transduced efficiently with the AAV2, 8, 9 and 10-vectorized MAB1 IgG and scFv-Fc constructs as well as the negative control MAB2 IgG 2 promoter LC/HC. For this, we transduced Human neuroblastoma cells that were differentiated into neurons using enriched synthetic medium supplemented by 10 μM retinoic acid and 2% fetal bovine serum at 37° C. under 5% CO2. About 200K cells were transduced in triplicate and incubated in 24 well microplates with 500 μL medium per well. The transduction was performed by adding about 100K genome copies/cell. Both vectorized MAB1 IgG and scFv-FC titers were determined by ELISA. A similar experiment was performed with hCMEC/D3 cells differentiated in blood brain barrier microvessels (see procedure in the following examples). Here, blood brain barrier cells were grown at either 50K or 100K per well, 37° C., 5% CO₂ and transduced by 50K genome copies (gc) per cell of AAV-vectorized MAB1 IgG IRES LC/HC construct. In both cell type cases, medium was changed every 3 days. In brief, 96 well microplates were coated with full length TDP-43 protein and then saturated with PBS buffer supplemented by 1% serum bovine albumin and 0.05% Tween. Samples were then added to the microplate wells and incubated for 1h at 37° C. In parallel, corresponding sample standards were added starting at the 2 μg/mL concentration and then diluted in 2-fold serial manner using the same buffer as the samples (PBS buffer supplemented by 1% serum bovine albumin and 0.05% Tween). Plates were washed with PBS buffer supplemented by 0.05% Tween. After this, an anti-human IgG Fc antibody labelled with horseradish peroxidase was added to the plates in PBS buffer supplemented by 1% serum bovine albumin and 0.05% Tween, and incubated for 1 h at 37° C. Plates were washed as above and horseradish peroxidase substrate (3,3′,5,5′— tetramethylbenzidine, called TMB) was added to the wells, incubated for about 5-10 min at room temperature and reactions were stopped using 0.5 M H2SO4. Samples were then read individually in a microplate reader at OD 450 nm. The collected data is presented in the FIG. 11 and compared to the above purified titers obtained with the same vectorized CHO transductions with the MAB1 lead constructs.

Observation. Both transduced commercially available cell types, differentiated neurons from a Human neuroblastoma cell line, and brain endothelial cell line (hCMECD/3), produced functional MAB1 IgG and scFv-Fc able to bind the full length TDP-43 protein (FIG. 11). Overall, the antibody and derivate are produced over time in large quantity (see middle panel, Day 18 for differentiated Human neuroblastoma cell line). They represent a mean of about 0.2-1 pg antibody/cell/day which is a robust cellular expression, similar to that reported for CHO cells grown at higher cell density (about 10 million/mL) expressing around 0.5-4 pg/cell/day. In these experiments, the titers obtained are higher for the AAV2-vectorized MAB1 IgG and scFv-Fc. In addition, the scFv-Fc titers are higher in each AAV8, 9 and 10 vectorization as compared to the vectorized IgG counterparts. The differentiated Human neuroblastoma cell line titers of the IRES construct were higher than the 2 promoter construct counterpart as a function of the capsid used for the vectorization. Finally, the vectorized MAB2 IgG 2 promoter LC/HC was not detected due to its property to bind another specific antigen than TDP-43 (see middle panel). In conclusion, the data collected confirms the earlier observation indicating that the IgG IRES constructs reach higher expression titers than the 2 promoters, using the LC/HC configuration.

TABLE 2
Nucleic acid sequences used in Vectorized antibody constructs:

| Sequence description | Nucleic acid sequence |
|---|---|
| Internal ribosome entry site | GCCCCTCTCCCTCCCCCCCCCCTAACGTTACT |
| (IRES) sequence | GGCCGAAGCCGCTTGGAATAAGGCCGGTGTG |
| | CGTTTGTCTATATGTTATTTTCCACCATATTGC |
| | CGTCTTTTGGCAATGTGAGGGCCCGGAAACC |
| | TGGCCCTGTCTTCTTGACGAGCATTCCTAGGG |
| | GTCTTTCCCCTCTCGCCAAAGGAATGCAAGGT |
| | CTGTTGAATGTCGTGAAGGAAGCAGTTCCTCT |
| | GGAAGCTTCTTGAAGACAAACAACGTCTGTAG |
| | CGACCCTTTGCAGGCAGCGGAACCCCCCACC |
| | TGGCGACAGGTGCCTCTGCGGCCAAAAGCCA |
| | CGTGTATAAGATACACCTGCAAAGGCGGCAC |
| | AACCCCAGTGCCACGTTGTGAGTTGGATAGTT |
| | GTGGAAAGAGTCAAATGGCTCTCCTCAAGCG |
| | TATTCAACAAGGGGCTGAAGGATGCCCAGAA |
| | GGTACCCCATTGTATGGGATCTGATCTGGGG |
| | CCTCGGTGCACATGCTTTACATGTGTTTAGTC |
| | GAGGTTAAAAAAACGTCTAGGCCCCCCGAAC |
| | CACGGGGACGTGGTTTTCCTTTGAAAAACACG |
| | ATGATAATATGGCCACAACC |
| | (SEQ ID NO: 1) |
| Mouse MAB2, promoter-LC-IRES-HC-WPRE | CCTGCAGGCAGCTGCGCGCTCGCTCGCTCAC |
| | TGAGGCCGCCCGGGCAAAGCCCGGGCGTCG |
| | GGCGACCTTTGGTCGCCCGGCCTCAGTGAGC |
| | GAGCGAGCGCGCAGAGAGGGAGTGGCCAAC |
| | TCCATCACTAGGGGTTCCTTCTAGACAACTTT |
| | GTATAGAAAAGTTGTAGTTATTAATAGTAATCA |
| | ATTACGGGGTCATTAGTTCATAGCCCATATAT |
| | GGAGTTCCGCGTTACATAACTTACGGTAAATG |
| | GCCCGCCTGGCTGACCGCCCAACGACCCCC |
| | GCCCATTGACGTCAATAATGACGTATGTTCCC |
| | ATAGTAACGCCAATAGGGACTTTCCATTGACG |
| | TCAATGGGTGGAGTATTTACGGTAAACTGCCC |
| | ACTTGGCAGTACATCAAGTGTATCATATGCCA |
| | AGTACGCCCCCTATTGACGTCAATGACGGTAA |
| | ATGGCCCGCCTGGCATTATGCCCAGTACATG |
| | ACCTTATGGGACTTTCCTACTTGGCAGTACAT |
| | CTACGTATTAGTCATCGCTATTACCATGGTGA |
| | TGCGGTTTTGGCAGTACATCAATGGGCGTGG |
| | ATAGCGGTTTGACTCACGGGGATTTCCAAGTC |
| | TCCACCCCATTGACGTCAATGGGAGTTTGTTT |
| | TGGCACCAAAATCAACGGGACTTTCCAAAATG |
| | TCGTAACAACTCCGCCCCATTGACGCAAATGG |
| | GCGGTAGGCGTGTACGGTGGGAGGTCTATAT |
| | AAGCAGAGCTGGTTTAGTGAACCGTCAGATC |
| | CAAGTTTGTACAAAAAAGCAGGCTGCCACCAT |
| | GGAGACAGATACACTGCTGCTGTGGGTGCTG |
| | CTCCTCTGGGTGCCAGGATCTACAGGCGACA |
| | TCGTGATGACCCAGAGCCACAAGTTCATGAG |
| | CACCAGCGTGGGCGACAGAGTGTCCATCACA |
| | TGCAAGGCCAGCCAGGACGTGAACACAGCCG |
| | TGGCTTGGTATCAGCAGAAGCCCGGCCATTC |
| | TCCTAAGCTGCTGATCTACAGCGCCAGCTTCA |
| | GATACACCGGCGTGCCCGATAGATTCACCGG |
| | CAACAGAAGCGGCACCGACTTCACCTTCACC |
| | ATCAGCTCTGTGCAGGCCGAGGATCTGGCCG |
| | TGTACTACTGTCAGCAGCACTACACCACACCT |
| | CCAACCTTCGGCGGAGGCACCAAGGTGGAAA |
| | TCAAGAGAGCTGACGCCGCTCCTACCGTGTC |
| | TATCTTCCCACCTAGCAGCGAGCAGCTGACAT |
| | CTGGCGGAGCCTCTGTCGTGTGCTTCCTGAA |
| | CAACTTCTACCCCAAGGACATCAACGTGAAGT |
| | GGAAGATCGACGGCAGCGAGAGACAGAACG |
| | GCGTGCTGAACTCTTGGACCGACCAGGACAG |
| | CAAGGACTCCACCTACAGCATGAGCAGCACC |
| | CTGACACTGACCAAGGACGAGTACGAGAGAC |
| | ACAACAGCTACACATGCGAGGCTACCCACAA |
| | GACCAGCACAAGCCCCATCGTGAAGTCCTTC |
| | AACAGAAACGAGTGCTGAACCCAGCTTTCTTG |
| | TACAAAGTGGGCCCCTCTCCCTCCCCCCCCC |
| | CTAACGTTACTGGCCGAAGCCGCTTGGAATAA |
| | GGCCGGTGTGCGTTTGTCTATATGTTATTTTC |
| | CACCATATTGCCGTCTTTTGGCAATGTGAGGG |
| | CCCGGAAACCTGGCCCTGTCTTCTTGACGAG |
| | CATTCCTAGGGGTCTTTCCCCTCTOGCCAAAG |
| | GAATGCAAGGTCTGTTGAATGTCGTGAAGGAA |
| | GCAGTTCCTCTGGAAGCTTCTTGAAGACAAAC |
| | AACGTCTGTAGCGACCCTTTGCAGGCAGCGG |
| | AACCCCCCACCTGGCGACAGGTGCCTCTGCG |
| | GCCAAAAGCCACGTGTATAAGATACACCTGCA |
| | AAGGCGGCACAACCCCAGTGCCACGTTGTGA |
| | GTTGGATAGTTGTGGAAAGAGTCAAATGGCTC |
| | TCCTCAAGCGTATTCAACAAGGGGCTGAAGG |
| | ATGCCCAGAAGGTACCCCATTGTATGGGATCT |
| | GATCTGGGGCCTCGGTGCACATGCTTTACAT |
| | GTGTTTAGTCGAGGTTAAAAAAACGTCTAGGC |
| | CCCCCGAACCACGGGGACGTGGTTTTCCTTT |
| | GAAAAACACGATGATAATATGGCCACAACCAT |
| | GGAGACAGATACACTGCTGCTGTGGGTGCTG |
| | CTCCTCTGGGTGCCAGGATCTACAGGCCAGG |
| | TTCAGCTGCAGCAGTCTGGACCTGAGCTGGT |
| | TAAGCCTGGCGCCTCTCTGAAGCTGAGCTGT |
| | ACCGCTTCCGGCTTCAACATCAAGGACACCTA |
| | CATCCACTGGGTCAAGCAGAGGCCTGAGCAG |
| | GGACTCGAGTGGATCGGCAGAATCTACCCCA |
| | CCAACGGCTACACCAGATACGACCCCAAGTT |
| | CCAGGACAAGGCCACCATCACAGCCGACACC |
| | AGCAGCAACACAGCCTATCTCCAGGTGTCCA |
| | GGCTGACCAGCGAGGACACAGCCGTGTACTA |
| | CTGCTCTAGATGGGGAGGCGACGGCTTCTAC |
| | GCCATGGATTATTGGGGACAGGGCGCCAGCG |
| | TGACAGTGTCTAGTGCCAAGACAACAGCCCC |
| | TAGCGTGTACCCTCTGGCTCCTGTGTGTGGC |
| | GACACAACAGGCAGCTCTGTGACACTGGGCT |
| | GTCTGGTCAAGGGCTACTTCCCCGAACCAGT |
| | GACACTGACCTGGAACAGCGGCTCTCTGTCT |
| | AGCGGCGTGCACACATTTCCAGCCGTGCTGC |
| | AGAGCGACCTGTACACACTGTCCTCTAGCGT |
| | GACCGTGACCAGCTCTACATGGCCCAGCCAG |
| | AGCATCACCTGTAACGTGGCCCATCCTGCCA |
| | GCAGCACCAAGGTGGACAAGAAGATCGAGCC |
| | TAGAGGCCCTACCATCAAGCCCTGTCCTCCAT |
| | GCAAGTGCCCCGCTCCTAATCTGCTCGGAGG |
| | CCCAAGCGTGTTCATCTTCCCACCTAAGATCA |
| | AGGACGTGCTGATGATCTCTCTGAGCCCCAT |
| | CGTGACCTGCGTGGTGGTGGATGTGTCTGAG |
| | GACGACCCTGACGTGCAGATCAGTTGGTTCG |
| | TGAACAACGTGGAAGTGCACACAGCCCAGAC |
| | ACAGACCCACAGAGAGGACTACAACAGCACC |
| | CTGAGAGTGGTGTCTGCCCTGCCTATCCAGC |
| | ACCAGGATTGGATGAGCGGCAAAGAATTCAA |
| | GTGCAAAGTGAACAACAAGGACCTGCCTGCT |
| | CCTATCGAGAGAACCATCAGCAAGCCCAAGG |
| | GCTCTGTCAGGGCTCCTCAGGTGTACGTTCT |
| | GCCACCTCCTGAGGAAGAGATGACCAAGAAA |
| | CAAGTGACCCTCACCTGTATGGTCACCGACTT |
| | CATGCCCGAGGACATCTACGTGGAATGGACC |
| | AACAACGGCAAGACCGAGCTGAACTACAAGA |
| | ACACCGAGCCTGTGCTGGACAGCGACGGCAG |
| | CTACTTCATGTACAGCAAGCTGCGCGTCGAG |
| | AAGAAGAACTGGGTCGAGAGAAACAGCTACA |
| | GCTGCTCCGTGGTGCACGAGGGACTGCACAA |
| | CCACCACACCACCAAGAGCTTCAGCAGAACC |
| | CCTGGCAAGTGACAACTTTATTATACATAGTT |
| | GGAATTCCGATAATCAACCTCTGGATTACAAA |
| | ATTTGTGAAAGATTGACTGGTATTCTTAACTAT |
| | GTTGCTCCTTTTACGCTATGTGGATACGCTGC |
| | TTTAATGCCTTTGTATCATGCTATTGCTTCCCG |
| | TATGGCTTTCATTTTCTCCTCCTTGTATAAATC |
| | CTGGTTGCTGTCTCTTTATGAGGAGTTGTGGC |
| | CCGTTGTCAGGCAACGTGGCGTGGTGTGCAC |
| | TGTGTTTGCTGACGCAACCCCCACTGGTTGG |
| | GGCATTGCCACCACCTGTCAGCTCCTTTCCG |
| | GGACTTTCGCTTTCCCCCTCCCTATTGCCACG |
| | GCGGAACTCATCGCCGCCTGCCTTGCCCGCT |
| | GCTGGACAGGGGCTCGGCTGTTGGGCACTGA |
| | CAATTCCGTGGTGTTGTCGGGGAAGCTGACG |
| | TCCTTTCCATGGCTGCTCGCCTGTGTTGCCAC |
| | CTGGATTCTGCGCGGGACGTCCTTCTGCTAC |
| | GTCCCTTCGGCCCTCAATCCAGCGGACCTTC |
| | CTTCCCGCGGCCTGCTGCCGGCTCTGCGGCC |
| | TCTTCCGCGTCTTCGCCTTCGCCCTCAGACGA |
| | GTCGGATCTCCCTTTGGGCCGCCTCCCCGCA |
| | TCGGGAATTCCTAGAGCTCGCTGATCAGCCT |
| | CGACTGTGCCTTCTAGTTGCCAGCCATCTGTT |
| | GTTTGCCCCTCCCCCGTGCCTTCCTTGACCCT |
| | GGAAGGTGCCACTCCCACTGTCCTTTCCTAAT |
| | AAAATGAGGAAATTGCATCGCATTGTCTGAGT |
| | AGGTGTCATTCTATTCTGGGGGGGGGGGG |
| | GGCAGGACAGCAAGGGGGAGGATTGGGAAG |
| | AGAATAGCAGGCATGCTGGGGAGGGCCGCA |
| | GGAACCCCTAGTGATGGAGTTGGCCACTCCC |
| | TCTCTGCGCGCTCGCTCGCTCACTGAGGCCG |
| | GGCGACCAAAGGTCGCCCGACGCCCGGGCT |
| | TTGCCCGGGGGGCCTCAGTGAGCGAGCGAG |
| | CGCGCAGCTGCCTGCAGG |
| | (SEQ ID NO: 2) |
| mouse MAB2-2 promoters IgG, consisting | CCTGCAGGCAGCTGCGCGCTCGCTCGCTCAC |
| of promoter 1 (CMV)-LC-polyA-promote | TGAGGCCGCCCGGGCAAAGCCCGGGCGTCG |
| 2 (CMV)-HC-WPRE-poly A | GGCGACCTTTGGTCGCCCGGCCTCAGTGAGC |
| | GAGCGAGCGCGCAGAGAGGGAGTGGCCAAC |
| | TCCATCACTAGGGGTTCCTTCTAGACAACTTT |
| | GTATAGAAAAGTTGTAGTTATTAATAGTAATCA |
| | ATTACGGGGTCATTAGTTCATAGCCCATATAT |
| | GGAGTTCCGCGTTACATAACTTACGGTAAATG |
| | GCCCGCCTGGCTGACCGCCCAACGACCCCC |
| | GCCCATTGACGTCAATAATGACGTATGTTCCC |
| | ATAGTAACGCCAATAGGGACTTTCCATTGACG |
| | TCAATGGGTGGAGTATTTACGGTAAACTGCCC |
| | ACTTGGCAGTACATCAAGTGTATCATATGCCA |
| | AGTACGCCCCCTATTGACGTCAATGACGGTAA |
| | ATGGCCCGCCTGGCATTATGCCCAGTACATG |
| | ACCTTATGGGACTTTCCTACTTGGCAGTACAT |
| | CTACGTATTAGTCATCGCTATTACCATGGTGA |
| | TGCGGTTTTGGCAGTACATCAATGGGCGTGG |
| | ATAGCGGTTTGACTCACGGGGATTTCCAAGTC |
| | TCCACCCCATTGACGTCAATGGGAGTTTGTTT |
| | TGGCACCAAAATCAACGGGACTTTCCAAAATG |
| | TCGTAACAACTCCGCCCCATTGACGCAAATGG |
| | GCGGTAGGCGTGTACGGTGGGAGGTCTATAT |
| | AAGCAGAGCTGGTTTAGTGAACCGTCAGATC |
| | CAAGTTTGTACAAAAAAGCAGGCTGCCACCAT |
| | GGAGACAGATACACTGCTGCTGTGGGTGCTG |
| | CTCCTCTGGGTGCCAGGATCTACAGGCGACA |
| | TCGTGATGACCCAGAGCCACAAGTTCATGAG |
| | CACCAGCGTGGGCGACAGAGTGTCCATCACA |
| | TGCAAGGCCAGCCAGGACGTGAACACAGCCG |
| | TGGCTTGGTATCAGCAGAAGCCCGGCCATTC |
| | TCCTAAGCTGCTGATCTACAGCGCCAGCTTCA |
| | GATACACCGGCGTGCCCGATAGATTCACCGG |
| | CAACAGAAGCGGCACCGACTTCACCTTCACC |
| | ATCAGCTCTGTGCAGGCCGAGGATCTGGCCG |
| | TGTACTACTGTCAGCAGCACTACACCACACCT |
| | CCAACCTTCGGCGGAGGCACCAAGGTGGAAA |
| | TCAAGAGAGCTGACGCCGCTCCTACCGTGTC |
| | TATCTTCCCACCTAGCAGCGAGCAGCTGACAT |
| | CTGGCGGAGCCTCTGTCGTGTGCTTCCTGAA |
| | CAACTTCTACCCCAAGGACATCAACGTGAAGT |
| | GGAAGATCGACGGCAGCGAGAGACAGAACG |
| | GCGTGCTGAACTCTTGGACCGACCAGGACAG |
| | CAAGGACTCCACCTACAGCATGAGCAGCACC |
| | CTGACACTGACCAAGGACGAGTACGAGAGAC |
| | ACAACAGCTACACATGCGAGGCTACCCACAA |
| | GACCAGCACAAGCCCCATCGTGAAGTCCTTC |
| | AACAGAAACGAGTGCTGACAGACATGATAAGA |
| | TACATTGATGAGTTTGGACAAACCACAACTAG |
| | AATGCAGTGAAAAAAATGCTTTATTTGTGAAAT |
| | TTGTGATGCTATTGCTTTATTTGTAACCATTAT |
| | AAGCTGCAATAAACAAGTTAACAACAACAATT |
| | GCATTCATTTTATGTTTCAGGTTCAGGGGGAG |
| | GTGTGGGAGGTTTTTTAAAGCAAGTAAAACCT |
| | CTACAAATGTGGTATAGTTATTAATAGTAATCA |
| | ATTACGGGGTCATTAGTTCATAGCCCATATAT |
| | GGAGTTCCGCGTTACATAACTTACGGTAAATG |
| | GCCCGCCTGGCTGACCGCCCAACGACCCCC |
| | GCCCATTGACGTCAATAATGACGTATGTTCCC |
| | ATAGTAACGCCAATAGGGACTTTCCATTGACG |
| | TCAATGGGTGGAGTATTTACGGTAAACTGCCC |
| | ACTTGGCAGTACATCAAGTGTATCATATGCCA |
| | AGTACGCCCCCTATTGACGTCAATGACGGTAA |
| | ATGGCCCGCCTGGCATTATGCCCAGTACATG |
| | ACCTTATGGGACTTTCCTACTTGGCAGTACAT |
| | CTACGTATTAGTCATCGCTATTACCATGGTGA |
| | TGCGGTTTTGGCAGTACATCAATGGGCGTGG |
| | ATAGCGGTTTGACTCACGGGGATTTCCAAGTC |
| | TCCACCCCATTGACGTCAATGGGAGTTTGTTT |
| | TGGCACCAAAATCAACGGGACTTTCCAAAATG |
| | TCGTAACAACTCCGCCCCATTGACGCAAATGG |
| | GCGGTAGGCGTGTACGGTGGGAGGTCTATAT |
| | AAGCAGAGCTGGTTTAGTGAACCGTCAGATCA |
| | CCCAGCTTTCTTGTACAAAGTGGGCCACCATG |
| | GAGACAGATACACTGCTGCTGTGGGTGCTGC |
| | TCCTCTGGGTGCCAGGATCTACAGGCCAGGT |
| | TCAGCTGCAGCAGTCTGGACCTGAGCTGGTT |
| | AAGCCTGGCGCCTCTCTGAAGCTGAGCTGTA |
| | CCGCTTCCGGCTTCAACATCAAGGACACCTAC |
| | ATCCACTGGGTCAAGCAGAGGCCTGAGCAGG |
| | GACTCGAGTGGATCGGCAGAATCTACCCCAC |
| | CAACGGCTACACCAGATACGACCCCAAGTTC |
| | CAGGACAAGGCCACCATCACAGCCGACACCA |
| | GCAGCAACACAGCCTATCTCCAGGTGTCCAG |
| | GCTGACCAGCGAGGACACAGCCGTGTACTAC |
| | TGCTCTAGATGGGGAGGCGACGGCTTCTACG |
| | CCATGGATTATTGGGGACAGGGCGCCAGCGT |
| | GACAGTGTCTAGTGCCAAGACAACAGCCCCT |
| | AGCGTGTACCCTCTGGCTCCTGTGTGTGGCG |
| | ACACAACAGGCAGCTCTGTGACACTGGGCTG |
| | TCTGGTCAAGGGCTACTTCCCCGAACCAGTG |
| | ACACTGACCTGGAACAGCGGCTCTCTGTCTA |
| | GCGGCGTGCACACATTTCCAGCCGTGCTGCA |
| | GAGCGACCTGTACACACTGTCCTCTAGCGTG |
| | ACCGTGACCAGCTCTACATGGCCCAGCCAGA |
| | GCATCACCTGTAACGTGGCCCATCCTGCCAG |
| | CAGCACCAAGGTGGACAAGAAGATCGAGCCT |
| | AGAGGCCCTACCATCAAGCCCTGTCCTCCAT |
| | GCAAGTGCCCCGCTCCTAATCTGCTCGGAGG |
| | CCCAAGCGTGTTCATCTTCCCACCTAAGATCA |
| | AGGACGTGCTGATGATCTCTCTGAGCCCCAT |
| | CGTGACCTGCGTGGTGGTGGATGTGTCTGAG |
| | GACGACCCTGACGTGCAGATCAGTTGGTTCG |
| | TGAACAACGTGGAAGTGCACACAGCCCAGAC |
| | ACAGACCCACAGAGAGGACTACAACAGCACC |
| | CTGAGAGTGGTGTCTGCCCTGCCTATCCAGC |
| | ACCAGGATTGGATGAGCGGCAAAGAATTCAA |
| | GTGCAAAGTGAACAACAAGGACCTGCCTGCT |
| | CCTATCGAGAGAACCATCAGCAAGCCCAAGG |
| | GCTCTGTCAGGGCTCCTCAGGTGTACGTTCT |
| | GCCACCTCCTGAGGAAGAGATGACCAAGAAA |
| | CAAGTGACCCTCACCTGTATGGTCACCGACTT |
| | CATGCCCGAGGACATCTACGTGGAATGGACC |
| | AACAACGGCAAGACCGAGCTGAACTACAAGA |
| | ACACCGAGCCTGTGCTGGACAGCGACGGCAG |
| | CTACTTCATGTACAGCAAGCTGCGCGTCGAG |
| | AAGAAGAACTGGGTCGAGAGAAACAGCTACA |
| | GCTGCTCCGTGGTGCACGAGGGACTGCACAA |
| | CCACCACACCACCAAGAGCTTCAGCAGAACC |
| | CCTGGCAAGTGACAACTTTATTATACATAGTT |
| | GGAATTCCTAGAGCTCGCTGATCAGCCTCGA |
| | CTGTGCCTTCTAGTTGCCAGCCATCTGTTGTT |
| | TGCCCCTCCCCCGTGCCTTCCTTGACCCTGG |
| | AAGGTGCCACTCCCACTGTCCTTTCCTAATAA |
| | AATGAGGAAATTGCATCGCATTGTCTGAGTAG |
| | GTGTCATTCTATTCTGGGGGGGGGGTGGGG |
| | CAGGACAGCAAGGGGGAGGATTGGGAAGAG |
| | AATAGCAGGCATGCTGGGGAGGGCCGCAGG |
| | AACCCCTAGTGATGGAGTTGGCCACTCCCTCT |
| | CTGCGCGCTCGCTCGCTCACTGAGGCCGGG |
| | CGACCAAAGGTCGCCCGACGCCCGGGCTTTG |
| | CCCGGGGGGCCTCAGTGAGCGAGCGAGCGC |
| | GCAGCTGCCTGCAGG |
| | (SEQ ID NO: 3) |
| Mouse MAB1, promoter-LC-IRES | CCTGCAGGCAGCTGCGCGCTCGCTCGCTCAC |
| shorter-HC-WPRE | TGAGGCCGCCCGGGCAAAGCCCGGGCGTCG |
| | GGCGACCTTTGGTCGCCCGGCCTCAGTGAGC |
| | GAGCGAGCGCGCAGAGAGGGAGTGGCCAAC |
| | TCCATCACTAGGGGTTCCTTCTAGACAACTTT |
| | GTATAGAAAAGTTGTAGTTATTAATAGTAATCA |
| | ATTACGGGGTCATTAGTTCATAGCCCATATAT |
| | GGAGTTCCGCGTTACATAACTTACGGTAAATG |
| | GCCCGCCTGGCTGACCGCCCAACGACCCCC |
| | GCCCATTGACGTCAATAATGACGTATGTTCCC |
| | ATAGTAACGCCAATAGGGACTTTCCATTGACG |
| | TCAATGGGTGGAGTATTTACGGTAAACTGCCC |
| | ACTTGGCAGTACATCAAGTGTATCATATGCCA |
| | AGTACGCCCCCTATTGACGTCAATGACGGTAA |
| | ATGGCCCGCCTGGCATTATGCCCAGTACATG |
| | ACCTTATGGGACTTTCCTACTTGGCAGTACAT |
| | CTACGTATTAGTCATCGCTATTACCATGGTGA |
| | TGCGGTTTTGGCAGTACATCAATGGGCGTGG |
| | ATAGCGGTTTGACTCACGGGGATTTCCAAGTC |
| | TCCACCCCATTGACGTCAATGGGAGTTTGTTT |
| | TGGCACCAAAATCAACGGGACTTTCCAAAATG |
| | TCGTAACAACTCCGCCCCATTGACGCAAATGG |
| | GCGGTAGGCGTGTACGGTGGGAGGTCTATAT |
| | AAGCAGAGCTGGTTTAGTGAACCGTCAGATC |
| | CAAGTTTGTACAAAAAAGCAGGCTGCCACCAT |
| | GGAGACAGATACACTGCTGCTGTGGGTGCTG |
| | CTCCTCTGGGTGCCAGGATCTACAGGCGACG |
| | TGGTCATGACACAGACCCCTCTGACACTGAG |
| | CGTGACCATCGGACAGCCTGCCAGCATCAGC |
| | TGCAAGAGCAGTCAGAGCCTGCTGCACAGCG |
| | ACGGCAAGACCTACCTGAACTGGCTGCTGCA |
| | AAGACCCGGCCAGTCTCCTAAGAGGCTGATC |
| | TACCTGGTGTCCAAGCTGGACAGCAGAATCC |
| | CCGACAGATTCACAGGCAGCGGCTCTGGCAC |
| | AGACTTCACCCTGAAGATCAGCAGAGTGGAA |
| | GCCGAGGACCTGGGCGTGTACTACTGTTGGC |
| | AGGGCACACACTTCCCTCACACATTCGGCGC |
| | TGGCACAAAGCTGGAACTGAAGAGAGCTGAC |
| | GCCGCTCCTACCGTGTCTATCTTCCCACCTAG |
| | CAGCGAGCAGCTGACATCTGGCGGAGCCTCT |
| | GTCGTGTGCTTCCTGAACAACTTCTACCCCAA |
| | GGACATCAACGTGAAGTGGAAGATCGACGGC |
| | AGCGAGAGACAGAACGGCGTGCTGAACTCTT |
| | GGACCGACCAGGACAGCAAGGACTCCACCTA |
| | CAGCATGAGCAGCACCCTGACACTGACCAAG |
| | GACGAGTACGAGAGACACAACAGCTACACAT |
| | GCGAGGCTACCCACAAGACCAGCACAAGCCC |
| | CATCGTGAAGTCCTTCAACAGAAACGAGTGCT |
| | GAGCCCCTCTCCCTCCCCCCCCCCTAACGTT |
| | ACTGGCCGAAGCCGCTTGGAATAAGGCCGGT |
| | GTGCGTTTGTCTATATGTTATTTTCCACCATAT |
| | TGCCGTCTTTTGGCAATGTGAGGGCCCGGAA |
| | ACCTGGCCCTGTCTTCTTGACGAGCATTCCTA |
| | GGGGTCTTTCCCCTCTCGCCAAAGGAATGCA |
| | AGGTCTGTTGAATGTCGTGAAGGAAGCAGTTC |
| | CTCTGGAAGCTTCTTGAAGACAAACAACGTCT |
| | GTAGCGACCCTTTGCAGGCAGCGGAACCCCC |
| | CACCTGGCGACAGGTGCCTCTGCGGCCAAAA |
| | GCCACGTGTATAAGATACACCTGCAAAGGCG |
| | GCACAACCCCAGTGCCACGTTGTGAGTTGGA |
| | TAGTTGTGGAAAGAGTCAAATGGCTCTCCTCA |
| | AGCGTATTCAACAAGGGGCTGAAGGATGCCC |
| | AGAAGGTACCCCATTGTATGGGATCTGATCTG |
| | GGGCCTCGGTGCACATGCTTTACATGTGTTTA |
| | GTCGAGGTTAAAAAAACGTCTAGGCCCCCCG |
| | AACCACGGGGACGTGGTTTTCCTTTGAAAAAC |
| | ACGATGATAATATGGAGACAGATACACTGCTG |
| | CTGTGGGTGCTGCTCCTCTGGGTGCCAGGAT |
| | CTACAGGCGAGGTTCAGCTGCAGCAGTCTGG |
| | ACCTGAGCTGGTTAAGCCTGGCGCCTCCGTG |
| | AAGATCAGCTGCAAGACAAGCGGCTTCACCTT |
| | CACCGAGTACAGCATGCACTGGGTCAAGCAG |
| | AGCCACGGCAAGAGCCTGGAATGGATCGGCG |
| | GCATCAACCCTAACAACGGCGGCACCAGCTA |
| | CAACCAGAAGTTCAAGGGCAAAGCCACACTG |
| | ACCGTGGACAAGAGCAGCAGCACCGCCTACA |
| | TGGAACTGAGAAGCCTGACCAGCGAGGACAG |
| | CGCCGTGTACTACTGTGCCAGAGAGTCTTGG |
| | GGCCAGGGCACAACCCTGACAGTCTCTTCTG |
| | CCAAGACAACAGCCCCTAGCGTGTACCCTCT |
| | GGCTCCTGTGTGTGGCGACACAACAGGCAGC |
| | TCTGTGACACTGGGCTGTCTGGTCAAGGGCT |
| | ACTTCCCCGAACCAGTGACACTGACCTGGAA |
| | CAGCGGCTCTCTGTCTAGCGGCGTGCACACA |
| | TTTCCAGCCGTGCTGCAGAGCGACCTGTACA |
| | CACTGTCCTCTAGCGTGACCGTGACCAGCTC |
| | TACATGGCCCAGCCAGAGCATCACCTGTAAC |
| | GTGGCCCATCCTGCCAGCAGCACCAAGGTGG |
| | ACAAGAAGATCGAGCCTAGAGGCCCTACCAT |
| | CAAGCCCTGTCCTCCATGCAAGTGCCCCGCT |
| | CCTAATCTGCTCGGAGGCCCAAGCGTGTTCA |
| | TCTTCCCACCTAAGATCAAGGACGTGCTGATG |
| | ATCTCTCTGAGCCCCATCGTGACCTGCGTGG |
| | TGGTGGATGTGTCTGAGGACGACCCTGACGT |
| | GCAGATCAGTTGGTTCGTGAACAACGTGGAA |
| | GTGCACACAGCCCAGACACAGACCCACAGAG |
| | AGGACTACAACAGCACCCTGAGAGTGGTGTC |
| | TGCCCTGCCTATCCAGCACCAGGATTGGATG |
| | AGCGGCAAAGAATTCAAGTGCAAAGTGAACAA |
| | CAAGGACCTGCCTGCTCCTATCGAGAGAACC |
| | ATCAGCAAGCCCAAGGGCTCTGTCAGGGCTC |
| | CTCAGGTGTACGTTCTGCCACCTCCTGAGGA |
| | AGAGATGACCAAGAAACAAGTGACCCTCACCT |
| | GTATGGTCACCGACTTCATGCCCGAGGACAT |
| | CTACGTGGAATGGACCAACAACGGCAAGACC |
| | GAGCTGAACTACAAGAACACCGAGCCTGTGC |
| | TGGACAGCGACGGCAGCTACTTCATGTACAG |
| | CAAGCTGCGCGTCGAGAAGAAGAACTGGGTC |
| | GAGAGAAACAGCTACAGCTGCTCCGTGGTGC |
| | ACGAGGGACTGCACAACCACCACACCACCAA |
| | GAGCTTCAGCAGAACCCCTGGCAAGTGAACC |
| | CAGCTTTCTTGTACAAAGTGGGAATTCCGATA |
| | ATCAACCTCTGGATTACAAAATTTGTGAAAGAT |
| | TGACTGGTATTCTTAACTATGTTGCTCCTTTTA |
| | CGCTATGTGGATACGCTGCTTTAATGCCTTTG |
| | TATCATGCTATTGCTTCCCGTATGGCTTTCATT |
| | TTCTCCTCCTTGTATAAATCCTGGTTGCTGTCT |
| | CTTTATGAGGAGTTGTGGCCCGTTGTCAGGC |
| | AACGTGGCGTGGTGTGCACTGTGTTTGCTGA |
| | CGCAACCCCCACTGGTTGGGGCATTGCCACC |
| | ACCTGTCAGCTCCTTTCCGGGACTTTCGCTTT |
| | CCCCCTCCCTATTGCCACGGCGGAACTCATC |
| | GCCGCCTGCCTTGCCCGCTGCTGGACAGGG |
| | GCTCGGCTGTTGGGCACTGACAATTCCGTGG |
| | TGTTGTCGGGGAAGCTGACGTCCTTTCCATG |
| | GCTGCTCGCCTGTGTTGCCACCTGGATTCTG |
| | CGCGGGACGTCCTTCTGCTACGTCCCTTCGG |
| | CCCTCAATCCAGCGGACCTTCCTTCCCGCGG |
| | CCTGCTGCCGGCTCTGCGGCCTCTTCCGCGT |
| | CTTCGCCTTCGCCCTCAGACGAGTCGGATCT |
| | CCCTTTGGGCCGCCTCCCCGCATCGGGAATT |
| | CCTAGAGCTCGCTGATCAGCCTCGACTGTGC |
| | CTTCTAGTTGCCAGCCATCTGTTGTTTGCCCC |
| | TCCCCCGTGCCTTCCTTGACCCTGGAAGGTG |
| | CCACTCCCACTGTCCTTTCCTAATAAAATGAG |
| | GAAATTGCATCGCATTGTCTGAGTAGGTGTCA |
| | TTCTATTCTGGGGGGTGGGGTGGGGCAGGAC |
| | AGCAAGGGGGAGGATTGGGAAGAGAATAGCA |
| | GGCATGCTGGGGAGGGCCGCAGGAACCCCT |
| | AGTGATGGAGTTGGCCACTCCCTCTCTGCGC |
| | GCTCGCTCGCTCACTGAGGCCGGGCGACCAA |
| | AGGTCGCCCGACGCCCGGGCTTTGCCCGGG |
| | CGGCCTCAGTGAGCGAGCGAGCGCGCAGCT |
| | GCCTGCAGG |
| | (SEQ ID NO: 4) |
| MAB1 promoter-LC-IRES-HC-WPRE-PolyA | CCTGCAGGCAGCTGCGCGCTCGCTCGCTCAC |
| | TGAGGCCGCCCGGGCAAAGCCCGGGCGTCG |
| | GGCGACCTTTGGTCGCCCGGCCTCAGTGAGC |
| | GAGCGAGCGCGCAGAGAGGGAGTGGCCAAC |
| | TCCATCACTAGGGGTTCCTTCTAGACAACTTT |
| | GTATAGAAAAGTTGTAGTTATTAATAGTAATCA |
| | ATTACGGGGTCATTAGTTCATAGCCCATATAT |
| | GGAGTTCCGCGTTACATAACTTACGGTAAATG |
| | GCCCGCCTGGCTGACCGCCCAACGACCCCC |
| | GCCCATTGACGTCAATAATGACGTATGTTCCC |
| | ATAGTAACGCCAATAGGGACTTTCCATTGACG |
| | TCAATGGGTGGAGTATTTACGGTAAACTGCCC |
| | ACTTGGCAGTACATCAAGTGTATCATATGCCA |
| | AGTACGCCCCCTATTGACGTCAATGACGGTAA |
| | ATGGCCCGCCTGGCATTATGCCCAGTACATG |
| | ACCTTATGGGACTTTCCTACTTGGCAGTACAT |
| | CTACGTATTAGTCATCGCTATTACCATGGTGA |
| | TGCGGTTTTGGCAGTACATCAATGGGCGTGG |
| | ATAGCGGTTTGACTCACGGGGATTTCCAAGTC |
| | TCCACCCCATTGACGTCAATGGGAGTTTGTTT |
| | TGGCACCAAAATCAACGGGACTTTCCAAAATG |
| | TCGTAACAACTCCGCCCCATTGACGCAAATGG |
| | GCGGTAGGCGTGTACGGTGGGAGGTCTATAT |
| | AAGCAGAGCTGGTTTAGTGAACCGTCAGATO |
| | CAAGTTTGTACAAAAAAGCAGGCTGCCACCAT |
| | GGAGACAGATACACTGCTGCTGTGGGTGCTG |
| | CTCCTCTGGGTGCCAGGATCTACAGGCGACG |
| | TGGTCATGACACAGACCCCTCTGACACTGTCC |
| | GTGACCATCGGACAGCCTGCCTCCATCTCCT |
| | GCAAGTCCTCTCAGTCCCTGCTGCACTCTGAC |
| | GGCAAGACCTACCTGAACTGGCTGCTGCAGA |
| | GGCCTGGCCAGAGTCCTAAGAGACTGATCTA |
| | CCTGGTGTCCAAGCTGGACTCTCGGATCCCT |
| | GACAGATTCACCGGCTCTGGCTCTGGCACCG |
| | ACTTCACCCTGAAGATCTCCAGAGTGGAAGC |
| | CGAGGACCTGGGCGTGTACTACTGTTGGCAG |
| | GGCACCCACTTTCCACACACCTTTGGCGCTG |
| | GCACAAAGCTGGAACTGAAGCGGACAGTGGC |
| | CGCTCCTTCCGTGTTCATCTTCCCACCTTCCG |
| | ACGAGCAGCTGAAGTCCGGCACAGCTTCTGT |
| | CGTGTGCCTGCTGAACAACTTCTACCCTCGG |
| | GAAGCCAAGGTGCAGTGGAAGGTGGACAATG |
| | CCCTGCAGTCCGGCAACTCCCAAGAGTCTGT |
| | GACCGAGCAGGACTCCAAGGACAGCACCTAC |
| | AGCCTGTCCTCCACACTGACCCTGTCCAAGG |
| | CCGACTACGAGAAGCACAAGGTGTACGCCTG |
| | CGAAGTGACCCATCAGGGCCTGTCTAGCCCT |
| | GTGACCAAGTCTTTCAACCGGGGCGAGTGTT |
| | GAACCCAGCTTTCTTGTACAAAGTGGGCCCCT |
| | CTCCCTCCCCCCCCCCTAACGTTACTGGCCG |
| | AAGCCGCTTGGAATAAGGCCGGTGTGCGTTT |
| | GTCTATATGTTATTTTCCACCATATTGCCGTCT |
| | TTTGGCAATGTGAGGGCCCGGAAACCTGGCC |
| | CTGTCTTCTTGACGAGCATTCCTAGGGGTCTT |
| | TCCCCTCTCGCCAAAGGAATGCAAGGTCTGTT |
| | GAATGTCGTGAAGGAAGCAGTTCCTCTGGAA |
| | GCTTCTTGAAGACAAACAACGTCTGTAGCGAC |
| | CCTTTGCAGGCAGCGGAACCCCCCACCTGGC |
| | GACAGGTGCCTCTGCGGCCAAAAGCCACGTG |
| | TATAAGATACACCTGCAAAGGCGGCACAACC |
| | CCAGTGCCACGTTGTGAGTTGGATAGTTGTG |
| | GAAAGAGTCAAATGGCTCTCCTCAAGCGTATT |
| | CAACAAGGGGCTGAAGGATGCCCAGAAGGTA |
| | CCCCATTGTATGGGATCTGATCTGGGGCCTC |
| | GGTGCACATGCTTTACATGTGTTTAGTCGAGG |
| | TTAAAAAAACGTCTAGGCCCCCCGAACCACG |
| | GGGACGTGGTTTTCCTTTGAAAAACACGATGA |
| | TAATATGGCCACAACCATGGAGACAGATACAC |
| | TGCTGCTGTGGGTGCTGCTCCTCTGGGTGCC |
| | AGGATCTACAGGCGAGGTTCAGCTGCAGCAG |
| | TCTGGACCTGAGCTGGTTAAGCCTGGCGCCT |
| | CCGTGAAGATCTCCTGCAAGACCTCTGGCTTC |
| | ACCTTCACCGAGTACTCCATGCACTGGGTCAA |
| | GCAGTCCCACGGCAAGTCCCTGGAATGGATC |
| | GGCGGCATCAACCCTAACAACGGCGGCACCT |
| | CCTACAACCAGAAGTTCAAGGGCAAAGCTAC |
| | CCTGACCGTGGACAAGTCCTCCTCCACCGCC |
| | TACATGGAACTGCGGTCCCTGACCTCTGAGG |
| | ACTCCGCCGTGTACTACTGCGCTAGAGAGTC |
| | TTGGGGCCAGGGCACCACACTGACAGTCTCT |
| | TCTGCTTCTACCAAGGGACCCAGCGTGTTCC |
| | CTCTGGCTCCTTCCAGCAAGTCTACCTCTGGC |
| | GGAACAGCTGCTCTGGGCTGCCTGGTCAAGG |
| | ACTACTTTCCTGAGCCTGTGACCGTGTCTTGG |
| | AACTCTGGCGCTCTGACATCCGGCGTGCACA |
| | CCTTTCCAGCTGTGCTGCAATCCAGCGGCCT |
| | GTACTCTCTGTCCTCCGTCGTGACCGTGCCTT |
| | CTAGCTCTCTGGGCACACAGACCTACATCTGC |
| | AATGTGAACCACAAGCCTTCCAACACCAAGGT |
| | GGACAAGAAGGTGGAACCCAAGTCCTGCGAC |
| | AAGACCCACACCTGTCCTCCATGTCCTGCTCC |
| | AGAACTGCTCGGCGGACCTTCCGTGTTCCTG |
| | TTTCCTCCAAAGCCTAAGGACACCCTGATGAT |
| | CTCTCGGACCCCTGAAGTGACCTGCGTGGTG |
| | GTGGATGTGTCTCACGAGGATCCCGAAGTGA |
| | AGTTCAATTGGTACGTGGACGGCGTGGAAGT |
| | GCACAACGCCAAGACCAAGCCTAGAGAGGAA |
| | CAGTACAACTCCACCTACAGAGTGGTGTCCGT |
| | GCTGACCGTGCTGCACCAGGATTGGCTGAAC |
| | GGCAAAGAGTACAAGTGCAAGGTGTCCAACA |
| | AGGCCCTGCCTGCTCCTATCGAAAAGACCAT |
| | CTCCAAGGCCAAGGGCCAGCCTAGGGAACCC |
| | CAGGTTTACACCTTGCCTCCATCTCGGGAAGA |
| | GATGACCAAGAACCAGGTGTCCCTGACCTGT |
| | CTCGTGAAGGGCTTCTACCCCTCCGATATCG |
| | CCGTGGAATGGGAGTCTAATGGCCAGCCTGA |
| | GAACAACTACAAGACAACCCCTCCTGTGCTG |
| | GACTCCGACGGCTCATTCTTCCTGTACTCCAA |
| | GCTGACAGTGGACAAGTCCAGATGGCAGCAG |
| | GGCAACGTGTTCTCCTGCTCCGTGATGCACG |
| | AGGCCCTGCACAATCACTACACCCAGAAGTC |
| | CCTGTCTCTGAGCCCCGGCAAGTAGCAACTTT |
| | ATTATACATAGTTGGAATTCCGATAATCAACCT |
| | CTGGATTACAAAATTTGTGAAAGATTGACTGG |
| | TATTCTTAACTATGTTGCTCCTTTTACGCTATG |
| | TGGATACGCTGCTTTAATGCCTTTGTATCATG |
| | CTATTGCTTCCCGTATGGCTTTCATTTTCTCCT |
| | CCTTGTATAAATCCTGGTTGCTGTCTCTTTATG |
| | AGGAGTTGTGGCCCGTTGTCAGGCAACGTGG |
| | CGTGGTGTGCACTGTGTTTGCTGACGCAACC |
| | CCCACTGGTTGGGGCATTGCCACCACCTGTC |
| | AGCTCCTTTCCGGGACTTTCGCTTTCCCCCTC |
| | CCTATTGCCACGGCGGAACTCATCGCCGCCT |
| | GCCTTGCCCGCTGCTGGACAGGGGCTCGGCT |
| | GTTGGGCACTGACAATTCCGTGGTGTTGTCG |
| | GGGAAGCTGACGTCCTTTCCATGGCTGCTCG |
| | CCTGTGTTGCCACCTGGATTCTGCGCGGGAC |
| | GTCCTTCTGCTACGTCCCTTCGGCCCTCAATC |
| | CAGCGGACCTTCCTTCCCGCGGCCTGCTGCC |
| | GGCTCTGCGGCCTCTTCCGCGTCTTCGCCTT |
| | CGCCCTCAGACGAGTCGGATCTCCCTTTGGG |
| | CCGCCTCCCCGCATCGGGAATTCCTAGAGCT |
| | CGCTGATCAGCCTCGACTGTGCCTTCTAGTTG |
| | CCAGCCATCTGTTGTTTGCCCCTCCCCCGTG |
| | CCTTCCTTGACCCTGGAAGGTGCCACTCCCA |
| | CTGTCCTTTCCTAATAAAATGAGGAAATTGCAT |
| | CGCATTGTCTGAGTAGGTGTCATTCTATTCTG |
| | GGGGGTGGGGTGGGGCAGGACAGCAAGGGG |
| | GAGGATTGGGAAGAGAATAGCAGGCATGCTG |
| | GGGAGGGCCGCAGGAACCCCTAGTGATGGA |
| | GTTGGCCACTCCCTCTCTGCGCGCTCGCTCG |
| | CTCACTGAGGCCGGGCGACCAAAGGTCGCCC |
| | GACGCCCGGGCTTTGCCCGGGGGGCCTCAG |
| | TGAGCGAGCGAGCGCGCAGCTGCCTGCAGG |
| | (SEQ ID NO: 5) |
| MAB1-2 promoters IgG, consisting of | CCTGCAGGCAGCTGCGCGCTCGCTCGCTCAC |
| promoter 1 (CMV)-LC-polyA-promoter 2 | TGAGGCCGCCCGGGCAAAGCCCGGGCGTCG |
| (CMV)-HC-WPRE-poly A | GGCGACCTTTGGTCGCCCGGCCTCAGTGAGC |
| | GAGCGAGCGCGCAGAGAGGGAGTGGCCAAC |
| | TCCATCACTAGGGGTTCCTTCTAGACAACTTT |
| | GTATAGAAAAGTTGTAGTTATTAATAGTAATCA |
| | ATTACGGGGTCATTAGTTCATAGCCCATATAT |
| | GGAGTTCCGCGTTACATAACTTACGGTAAATG |
| | GCCCGCCTGGCTGACCGCCCAACGACCCCC |
| | GCCCATTGACGTCAATAATGACGTATGTTCCC |
| | ATAGTAACGCCAATAGGGACTTTCCATTGACG |
| | TCAATGGGTGGAGTATTTACGGTAAACTGCCC |
| | ACTTGGCAGTACATCAAGTGTATCATATGCCA |
| | AGTACGCCCCCTATTGACGTCAATGACGGTAA |
| | ATGGCCCGCCTGGCATTATGCCCAGTACATG |
| | ACCTTATGGGACTTTCCTACTTGGCAGTACAT |
| | CTACGTATTAGTCATCGCTATTACCATGGTGA |
| | TGCGGTTTTGGCAGTACATCAATGGGCGTGG |
| | ATAGCGGTTTGACTCACGGGGATTTCCAAGTC |
| | TCCACCCCATTGACGTCAATGGGAGTTTGTTT |
| | TGGCACCAAAATCAACGGGACTTTCCAAAATG |
| | TCGTAACAACTCCGCCCCATTGACGCAAATGG |
| | GCGGTAGGCGTGTACGGTGGGAGGTCTATAT |
| | AAGCAGAGCTGGTTTAGTGAACCGTCAGATC |
| | CAAGTTTGTACAAAAAAGCAGGCTGCCACCAT |
| | GGAGACAGATACACTGCTGCTGTGGGTGCTG |
| | CTCCTCTGGGTGCCAGGATCTACAGGCGACG |
| | TGGTCATGACACAGACCCCTCTGACACTGTCC |
| | GTGACCATCGGACAGCCTGCCTCCATCTCCT |
| | GCAAGTCCTCTCAGTCCCTGCTGCACTCTGAC |
| | GGCAAGACCTACCTGAACTGGCTGCTGCAGA |
| | GGCCTGGCCAGAGTCCTAAGAGACTGATCTA |
| | CCTGGTGTCCAAGCTGGACTCTCGGATCCCT |
| | GACAGATTCACCGGCTCTGGCTCTGGCACCG |
| | ACTTCACCCTGAAGATCTCCAGAGTGGAAGC |
| | CGAGGACCTGGGCGTGTACTACTGTTGGCAG |
| | GGCACCCACTTTCCACACACCTTTGGCGCTG |
| | GCACAAAGCTGGAACTGAAGCGGACAGTGGC |
| | CGCTCCTTCCGTGTTCATCTTCCCACCTTCCG |
| | ACGAGCAGCTGAAGTCCGGCACAGCTTCTGT |
| | CGTGTGCCTGCTGAACAACTTCTACCCTCGG |
| | GAAGCCAAGGTGCAGTGGAAGGTGGACAATG |
| | CCCTGCAGTCCGGCAACTCCCAAGAGTCTGT |
| | GACCGAGCAGGACTCCAAGGACAGCACCTAC |
| | AGCCTGTCCTCCACACTGACCCTGTCCAAGG |
| | CCGACTACGAGAAGCACAAGGTGTACGCCTG |
| | CGAAGTGACCCATCAGGGCCTGTCTAGCCCT |
| | GTGACCAAGTCTTTCAACCGGGGCGAGTGTT |
| | GACAGACATGATAAGATACATTGATGAGTTTG |
| | GACAAACCACAACTAGAATGCAGTGAAAAAAA |
| | TGCTTTATTTGTGAAATTTGTGATGCTATTGCT |
| | TTATTTGTAACCATTATAAGCTGCAATAAACAA |
| | GTTAACAACAACAATTGCATTCATTTTATGTTT |
| | CAGGTTCAGGGGGAGGTGTGGGAGGTTTTTT |
| | AAAGCAAGTAAAACCTCTACAAATGTGGTATA |
| | GTTATTAATAGTAATCAATTACGGGGTCATTAG |
| | TTCATAGCCCATATATGGAGTTCCGCGTTACA |
| | TAACTTACGGTAAATGGCCCGCCTGGCTGAC |
| | CGCCCAACGACCCCCGCCCATTGACGTCAAT |
| | AATGACGTATGTTCCCATAGTAACGCCAATAG |
| | GGACTTTCCATTGACGTCAATGGGTGGAGTAT |
| | TTACGGTAAACTGCCCACTTGGCAGTACATCA |
| | AGTGTATCATATGCCAAGTACGCCCCCTATTG |
| | ACGTCAATGACGGTAAATGGCCCGCCTGGCA |
| | TTATGCCCAGTACATGACCTTATGGGACTTTC |
| | CTACTTGGCAGTACATCTACGTATTAGTCATC |
| | GCTATTACCATGGTGATGCGGTTTTGGCAGTA |
| | CATCAATGGGCGTGGATAGCGGTTTGACTCA |
| | CGGGGATTTCCAAGTCTCCACCCCATTGACGT |
| | CAATGGGAGTTTGTTTTGGCACCAAAATCAAC |
| | GGGACTTTCCAAAATGTCGTAACAACTCCGCC |
| | CCATTGACGCAAATGGGGGGTAGGCGTGTAC |
| | GGTGGGAGGTCTATATAAGCAGAGCTGGTTT |
| | AGTGAACCGTCAGATCACCCAGCTTTCTTGTA |
| | CAAAGTGGGCCACCATGGAGACAGATACACT |
| | GCTGCTGTGGGTGCTGCTCCTCTGGGTGCCA |
| | GGATCTACAGGCGAGGTTCAGCTGCAGCAGT |
| | CTGGACCTGAGCTGGTTAAGCCTGGCGCCTC |
| | CGTGAAGATCTCCTGCAAGACCTCTGGCTTCA |
| | CCTTCACCGAGTACTCCATGCACTGGGTCAA |
| | GCAGTCCCACGGCAAGTCCCTGGAATGGATC |
| | GGCGGCATCAACCCTAACAACGGCGGCACCT |
| | CCTACAACCAGAAGTTCAAGGGCAAAGCTAC |
| | CCTGACCGTGGACAAGTCCTCCTCCACCGCC |
| | TACATGGAACTGCGGTCCCTGACCTCTGAGG |
| | ACTCCGCCGTGTACTACTGCGCTAGAGAGTC |
| | TTGGGGCCAGGGCACCACACTGACAGTCTCT |
| | TCTGCTTCTACCAAGGGACCCAGCGTGTTCC |
| | CTCTGGCTCCTTCCAGCAAGTCTACCTCTGGC |
| | GGAACAGCTGCTCTGGGCTGCCTGGTCAAGG |
| | ACTACTTTCCTGAGCCTGTGACCGTGTCTTGG |
| | AACTCTGGCGCTCTGACATCCGGCGTGCACA |
| | CCTTTCCAGCTGTGCTGCAATCCAGCGGCCT |
| | GTACTCTCTGTCCTCCGTCGTGACCGTGCCTT |
| | CTAGCTCTCTGGGCACACAGACCTACATCTGC |
| | AATGTGAACCACAAGCCTTCCAACACCAAGGT |
| | GGACAAGAAGGTGGAACCCAAGTCCTGCGAC |
| | AAGACCCACACCTGTCCTCCATGTCCTGCTCC |
| | AGAACTGCTCGGCGGACCTTCCGTGTTCCTG |
| | TTTCCTCCAAAGCCTAAGGACACCCTGATGAT |
| | CTCTCGGACCCCTGAAGTGACCTGCGTGGTG |
| | GTGGATGTGTCTCACGAGGATCCCGAAGTGA |
| | AGTTCAATTGGTACGTGGACGGCGTGGAAGT |
| | GCACAACGCCAAGACCAAGCCTAGAGAGGAA |
| | CAGTACAACTCCACCTACAGAGTGGTGTCCGT |
| | GCTGACCGTGCTGCACCAGGATTGGCTGAAC |
| | GGCAAAGAGTACAAGTGCAAGGTGTCCAACA |
| | AGGCCCTGCCTGCTCCTATCGAAAAGACCAT |
| | CTCCAAGGCCAAGGGCCAGCCTAGGGAACCC |
| | CAGGTTTACACCTTGCCTCCATCTCGGGAAGA |
| | GATGACCAAGAACCAGGTGTCCCTGACCTGT |
| | CTCGTGAAGGGCTTCTACCCCTCCGATATCG |
| | CCGTGGAATGGGAGTCTAATGGCCAGCCTGA |
| | GAACAACTACAAGACAACCCCTCCTGTGCTG |
| | GACTCCGACGGCTCATTCTTCCTGTACTCCAA |
| | GCTGACAGTGGACAAGTCCAGATGGCAGCAG |
| | GGCAACGTGTTCTCCTGCTCCGTGATGCACG |
| | AGGCCCTGCACAATCACTACACCCAGAAGTC |
| | CCTGTCTCTGAGCCCCGGCAAGTAGCAACTTT |
| | ATTATACATAGTTGGAATTCCTAGAGCTCGCT |
| | GATCAGCCTCGACTGTGCCTTCTAGTTGCCAG |
| | CCATCTGTTGTTTGCCCCTCCCCCGTGCCTTC |
| | CTTGACCCTGGAAGGTGCCACTCCCACTGTC |
| | CTTTCCTAATAAAATGAGGAAATTGCATCGCA |
| | TTGTCTGAGTAGGTGTCATTCTATTCTGGGGG |
| | GTGGGGTGGGGCAGGACAGCAAGGGGGAGG |
| | ATTGGGAAGAGAATAGCAGGCATGCTGGGGA |
| | GGGCCGCAGGAACCCCTAGTGATGGAGTTGG |
| | CCACTCCCTCTCTGCGCGCTCGCTCGCTCAC |
| | TGAGGCCGGGCGACCAAAGGTCGCCCGACG |
| | CCCGGGCTTTGCCCGGGGGGCCTCAGTGAG |
| | CGAGCGAGCGCGCAGCTGCCTGCAGG |
| | (SEQ ID NO: 6) |
| MAB1, promoter-scFv-Fc-WPRE | cctgcaggcagctgcgcgctcgctcgctcactgaggccgcccggg |
| | caaagcccgggcgtcgggcgacctttggtcgcccggcctcagtga |
| | gcgagcgagcgcgcagagagggagtggccaactccatcactag |
| | gggttccttctagacaactttgtatagaaaagttgtagttattaatagta |
| | atcaattacggggtcattagttcatagcccatatatggagttccgcgtt |
| | acataacttacggtaaatggcccgcctggctgaccgcccaacgac |
| | ccccgcccattgacgtcaataatgacgtatgttcccatagtaacgcc |
| | aatagggactttccattgacgtcaatgggtggagtatttacggtaaa |
| | ctgcccacttggcagtacatcaagtgtatcatatgccaagtacgccc |
| | cctattgacgtcaatgacggtaaatggcccgcctggcattatgccca |
| | gtacatgaccttatgggactttcctacttggcagtacatctacgtatta |
| | gtcatcgctattaccatggtgatgcggttttggcagtacatcaatggg |
| | cgtggatagcggtttgactcacggggatttccaagtctccaccccatt |
| | gacgtcaatgggagtttgttttggcaccaaaatcaacgggactttcc |
| | aaaatgtcgtaacaactccgccccattgacgcaaatgggcggtag |
| | gcgtgtacggtgggaggtctatataagcagagctggtttagtgaac |
| | cgtcagatccaagtttgtacaaaaaagcaggctgccaccatggag |
| | acagatacactgctgctgtgggtgctgctcctctgggtgccaggatc |
| | tacaggcgaggttcagctgcagcagtctggacctgagctggttaag |
| | cctggcgcctccgtgaagatctcctgcaagacctctggcttcaccttc |
| | accgagtactccatgcactgggtcaagcagtcccacggcaagtcc |
| | ctggaatggatcggcggcatcaaccctaacaacggcggcacctc |
| | ctacaaccagaagttcaagggcaaagctaccctgaccgtggaca |
| | agtcctcctccaccgcctacatggaactgcggtccctgacctctgag |
| | gactccgccgtgtactactgcgctagagagtcttggggccagggc |
| | accacactgacagtctcttctggaggcggaggatctggcggaggt |
| | ggaagtggcggaggcggatctgacgtggtcatgacacagacccc |
| | tctgacactgtccgtgaccatcggacagcctgcctccatctcctgca |
| | agtcctctcagtccctgctgcactctgacggcaagacctacctgaa |
| | ctggctgctgcagaggcctggccagagtcctaagagactgatcta |
| | cctggtgtccaagctggactctcggatccctgacagattcaccggct |
| | ctggctctggcaccgacttcaccctgaagatctccagagtggaagc |
| | cgaggacctgggcgtgtactactgttggcagggcacccactttcca |
| | cacacctttggcgctggcacaaagctggaactgaagggaggcgg |
| | aggatctgacaagacccacacctgtcctccatgtcctgctccagaa |
| | ctgctcggcggaccttccgtgttcctgtttcctccaaagcctaaggac |
| | accctgatgatctctcggacccctgaagtgacctgcgtggtggtgg |
| | atgtgtctcacgaggatcccgaagtgaagttcaattggtacgtggac |
| | ggcgtggaagtgcacaacgccaagaccaagcctagagaggaa |
| | cagtacaactccacctacagagtggtgtccgtgctgaccgtgctgc |
| | accaggattggctgaacggcaaagagtacaagtgcaaggtgtcc |
| | aacaaggccctgcctgctcctatcgaaaagaccatctccaaggcc |
| | aagggccagcctagggaaccccaggtttacaccttgcctccatctc |
| | gggaagagatgaccaagaaccaggtgtccctgacctgtctcgtga |
| | agggcttctacccctccgatatcgccgtggaatgggagtctaatgg |
| | ccagcctgagaacaactacaagacaacccctcctgtgctggactc |
| | cgacggctcattcttcctgtactccaagctgacagtggacaagtcca |
| | gatggcagcagggcaacgtgttctcctgctccgtgatgcacgagg |
| | ccctgcacaatcactacacccagaagtccctgtctctgagccccgg |
| | caagtagacccagctttcttgtacaaagtgggaattccgataatcaa |
| | cctctggattacaaaatttgtgaaagattgactggtattcttaactatgt |
| | tgctccttttacgctatgtggatacgctgctttaatgcctttgtatcatgct |
| | attgcttcccgtatggctttcattttctcctccttgtataaatcctggttgct |
| | gtctctttatgaggagttgtggcccgttgtcaggcaacgtggcgtggt |
| | gtgcactgtgtttgctgacgcaacccccactggttggggcattgcca |
| | ccacctgtcagctcctttccgggactttcgctttccccctccctattgcc |
| | acggcggaactcatcgccgcctgccttgcccgctgctggacaggg |
| | gctcggctgttgggcactgacaattccgtggtgttgtcggggaagct |
| | gacgtcctttccatggctgctcgcctgtgttgccacctggattctgcgc |
| | gggacgtccttctgctacgtcccttcggccctcaatccagcggacctt |
| | ccttcccgcggcctgctgccggctctgcggcctcttccgcgtcttcgc |
| | cttcgccctcagacgagtcggatctccctttgggccgcctccccgca |
| | tcgggaattcctagagctcgctgatcagcctcgactgtgccttctagtt |
| | gccagccatctgttgtttgcccctcccccgtgccttccttgaccctgga |
| | aggtgccactcccactgtcctttcctaataaaatgaggaaattgcat |
| | cgcattgtctgagtaggtgtcattctattctggggggggggtggggc |
| | aggacagcaagggggaggattgggaagagaatagcaggcatg |
| | ctggggagggccgcaggaacccctagtgatggagttggccactc |
| | cctctctgcgcgctcgctcgctcactgaggccgggcgaccaaagg |
| | tcgcccgacgcccgggctttgcccgggcggcctcagtgagcgag |
| | cgagcgcgcagctgcctgcagg |
| | (SEQ ID NO: 7) |
| Internal ribosome entry site shorter | GCCCCTCTCCCTCCCCCCCCCCTAACGTTACT |
| sequence (IRES shorter) | GGCCGAAGCCGCTTGGAATAAGGCCGGTGTG |
| | CGTTTGTCTATATGTTATTTTCCACCATATTGC |
| | CGTCTTTTGGCAATGTGAGGGCCCGGAAACC |
| | TGGCCCTGTCTTCTTGACGAGCATTCCTAGGG |
| | GTCTTTCCCCTCTCGCCAAAGGAATGCAAGGT |
| | CTGTTGAATGTCGTGAAGGAAGCAGTTCCTCT |
| | GGAAGCTTCTTGAAGACAAACAACGTCTGTAG |
| | CGACCCTTTGCAGGCAGCGGAACCCCCCACC |
| | TGGCGACAGGTGCCTCTGCGGCCAAAAGCCA |
| | CGTGTATAAGATACACCTGCAAAGGCGGCAC |
| | AACCCCAGTGCCACGTTGTGAGTTGGATAGTT |
| | GTGGAAAGAGTCAAATGGCTCTCCTCAAGCG |
| | TATTCAACAAGGGGCTGAAGGATGCCCAGAA |
| | GGTACCCCATTGTATGGGATCTGATCTGGGG |
| | CCTCGGTGCACATGCTTTACATGTGTTTAGTC |
| | GAGGTTAAAAAAACGTCTAGGCCCCCCGAAC |
| | CACGGGGACGTGGTTTTCCTTTGAAAAACACG |
| | ATGATAAT |
| | (SEQ ID NO: 8) |
| MAB1 FAB promoter-LC-IRES-HC-WPRE- | cctgcaggcagctgcgcgctcgctcgctcactgaggccgcccgggc |
| PolyA | aaagcccgggcgtcgggcgacctttggtcgcccggcctcagtgagc |
| | gagcgagcgcgcagagagggagtggccaactccatcactaggggt |
| | tccttctagacaactttgtatagaaaagttgtagttattaatagtaat |
| | caattacggggtcattagttcatagcccatatatggagttccgcgtta |
| | cataacttacggtaaatggcccgcctggctgaccgcccaacgaccc |
| | ccgcccattgacgtcaataatgacgtatgttcccatagtaacgccaa |
| | tagggactttccattgacgtcaatgggtggagtatttacggtaaact |
| | gcccacttggcagtacatcaagtgtatcatatgccaagtacgccccc |
| | tattgacgtcaatgacggtaaatggcccgcctggcattatgcccagt |
| | acatgaccttatgggactttcctacttggcagtacatctacgtattag |
| | tcatcgctattaccatggtgatgcggttttggcagtacatcaatgggc |
| | gtggatagcggtttgactcacggggatttccaagtctccaccccatt |
| | gacgtcaatgggagtttgttttggcaccaaaatcaacgggactttcc |
| | aaaatgtcgtaacaactccgccccattgacgcaaatgggcggtagg |
| | cgtgtacggtgggaggtctatataagcagagctggtttagtgaaccg |
| | tcagatccaagtttgtacaaaaaagcaggctgccaccatggagaca |
| | gatacactgctgctgtgggtgctgctcctctgggtgccaggatctaca |
| | ggcgacgtggtcatgacacagacccctctgacactgtccgtgaccat |
| | cggacagcctgcctccatctcctgcaagtcctctcagtccctgctgca |
| | ctctgacggcaagacctacctgaactggctgctgcagaggcctggc |
| | cagagtcctaagagactgatctacctggtgtccaagctggactctcg |
| | gatccctgacagattcaccggctctggctctggcaccgacttcaccct |
| | gaagatctccagagtggaagccgaggacctgggcgtgtactactgt |
| | tggcagggcacccactttccacacacctttggcgctggcacaaagct |
| | ggaactgaagcggacagtggccgctccttccgtgttcatcttcccac |
| | cttccgacgagcagctgaagtccggcacagcttctgtcgtgtgcctg |
| | ctgaacaacttctaccctcgggaagccaaggtgcagtggaaggtgg |
| | acaatgccctgcagtccggcaactcccaagagtctgtgaccgagca |
| | ggactccaaggacagcacctacagcctgtcctccacactgaccctgt |
| | ccaaggccgactacgagaagcacaaggtgtacgcctgcgaagtga |
| | cccatcagggcctgtctagccctgtgaccaagtctttcaaccggggc |
| | gagtgttgaacccagctttcttgtacaaagtgggcccctctccctccc |
| | ccccccctaacgttactggccgaagccgcttggaataaggccggtgt |
| | gcgtttgtctatatgttattttccaccatattgccgtcttttggcaatgt |
| | gagggcccggaaacctggccctgtcttcttgacgagcattcctaggg |
| | gtctttcccctctcgccaaaggaatgcaaggtctgttgaatgtcgtga |
| | aggaagcagttcctctggaagcttcttgaagacaaacaacgtctgt |
| | agcgaccctttgcaggcagcggaaccccccacctggcgacaggtgc |
| | ctctgcggccaaaagccacgtgtataagatacacctgcaaaggcgg |
| | cacaaccccagtgccacgttgtgagttggatagttgtggaaagagtc |
| | aaatggctctcctcaagcgtattcaacaaggggctgaaggatgccc |
| | agaaggtaccccattgtatgggatctgatctggggcctcggtgcaca |
| | tgctttacatgtgtttagtcgaggttaaaaaaacgtctaggccccccg |
| | aaccacggggacgtggttttcctttgaaaaacacgatgataatatgg |
| | ccacaaccatggagacagatacactgctgctgtgggtgctgctcctc |
| | tgggtgccaggatctacaggcgaggttcagctgcagcagtctggac |
| | ctgagctggttaagcctggcgcctccgtgaagatctcctgcaagacc |
| | tctggcttcaccttcaccgagtactccatgcactgggtcaagcagtcc |
| | cacggcaagtccctggaatggatcggcggcatcaaccctaacaacg |
| | gcggcacctcctacaaccagaagttcaagggcaaagctaccctgac |
| | cgtggacaagtcctcctccaccgcctacatggaactgcggtccctga |
| | cctctgaggactccgccgtgtactactgcgctagagagtcttggggc |
| | cagggcaccacactgacagtctcttctgcttctaccaagggacccag |
| | cgtgttccctctggctccttccagcaagtctacctctggcggaacagc |
| | tgctctgggctgcctggtcaaggactactttcctgagcctgtgaccgt |
| | gtcttggaactctggcgctctgacatccggcgtgcacacctttccagc |
| | tgtgctgcaatccagcggcctgtactctctgtcctccgtcgtgaccgt |
| | gccttctagctctctgggcacacagacctacatctgcaatgtgaacc |
| | acaagccttccaacaccaaggtggacaagaaggtggaacccaagt |
| | cctgcggctcccaccaccatcaccatcattagcaactttattatacat |
| | agttggaattccgataatcaacctctggattacaaaatttgtgaaag |
| | attgactggtattcttaactatgttgctccttttacgctatgtggatac |
| | gctgctttaatgcctttgtatcatgctattgcttcccgtatggctttcat |
| | tttctcctccttgtataaatcctggttgctgtctctttatgaggagttgt |
| | ggcccgttgtcaggcaacgtggcgtggtgtgcactgtgtttgctgac |
| | gcaacccccactggttggggcattgccaccacctgtcagctcctttcc |
| | gggactttcgctttccccctccctattgccacggcggaactcatcgcc |
| | gcctgccttgcccgctgctggacaggggctcggctgttgggcactga |
| | caattccgtggtgttgtcggggaagctgacgtcctttccatggctgct |
| | cgcctgtgttgccacctggattctgcgcgggacgtccttctgctacgt |
| | cccttcggccctcaatccagcggaccttccttcccgcggcctgctgcc |
| | ggctctgcggcctcttccgcgtcttcgccttcgccctcagacgagtcg |
| | gatctccctttgggccgcctccccgcatcgggaattcctagagctcgc |
| | tgatcagcctcgactgtgccttctagttgccagccatctgttgtttgcc |
| | cctcccccgtgccttccttgaccctggaaggtgccactcccactgtcc |
| | tttcctaataaaatgaggaaattgcatcgcattgtctgagtaggtgtc |
| | attctattctggggggtggggtggggcaggacagcaagggggagg |
| | attgggaagagaatagcaggcatgctggggagggccgcaggaacc |
| | cctagtgatggagttggccactccctctctgcgcgctcgctcgctcac |
| | tgaggccgggcgaccaaaggtcgcccgacgcccgggctttgcccgg |
| | gcggcctcagtgagcgagcgagcgcgcagctgcctgcagg |
| | (SEQ ID NO: 9) |
| MAB1 Fab-2 promoters IgG, consisting | cctgcaggcagctgcgcgctcgctcgctcactgaggccgcccgggc |
| of promoter 1 (CMV)-HC-polyA-promoter | aaagcccgggcgtcgggcgacctttggtcgcccggcctcagtgagc |
| 2 (CMV)-LC-WPRE-poly A | gagcgagcgcgcagagagggagtggccaactccatcactaggggt |
| | tccttctagacaactttgtatagaaaagttgtagttattaatagtaat |
| | caattacggggtcattagttcatagcccatatatggagttccgcgtta |
| | cataacttacggtaaatggcccgcctggctgaccgcccaacgaccc |
| | ccgcccattgacgtcaataatgacgtatgttcccatagtaacgccaa |
| | tagggactttccattgacgtcaatgggtggagtatttacggtaaact |
| | gcccacttggcagtacatcaagtgtatcatatgccaagtacgccccc |
| | tattgacgtcaatgacggtaaatggcccgcctggcattatgcccagt |
| | acatgaccttatgggactttcctacttggcagtacatctacgtattag |
| | tcatcgctattaccatggtgatgcggttttggcagtacatcaatgggc |
| | gtggatagcggtttgactcacggggatttccaagtctccaccccatt |
| | gacgtcaatgggagtttgttttggcaccaaaatcaacgggactttcc |
| | aaaatgtcgtaacaactccgccccattgacgcaaatgggcggtagg |
| | cgtgtacggtgggaggtctatataagcagagctggtttagtgaaccg |
| | tcagatccaagtttgtacaaaaaagcaggctgccaccatggagaca |
| | gatacactgctgctgtgggtgctgctcctctgggtgccaggatctaca |
| | ggcgaggttcagctgcagcagtctggacctgagctggttaagcctg |
| | gcgcctccgtgaagatctcctgcaagacctctggcttcaccttcaccg |
| | agtactccatgcactgggtcaagcagtcccacggcaagtccctgga |
| | atggatcggcggcatcaaccctaacaacggcggcacctcctacaac |
| | cagaagttcaagggcaaagctaccctgaccgtggacaagtcctcct |
| | ccaccgcctacatggaactgcggtccctgacctctgaggactccgcc |
| | gtgtactactgcgctagagagtcttggggccagggcaccacactga |
| | cagtctcttctgcttctaccaagggacccagcgtgttccctctggctc |
| | cttccagcaagtctacctctggcggaacagctgctctgggctgcctg |
| | gtcaaggactactttcctgagcctgtgaccgtgtcttggaactctggc |
| | gctctgacatccggcgtgcacacctttccagctgtgctgcaatccag |
| | cggcctgtactctctgtcctccgtcgtgaccgtgccttctagctctctg |
| | ggcacacagacctacatctgcaatgtgaaccacaagccttccaaca |
| | ccaaggtggacaagaaggtggaacccaagtcctgcggctcccacc |
| | accatcaccatcattagcagacatgataagatacattgatgagtttg |
| | gacaaaccacaactagaatgcagtgaaaaaaatgctttatttgtga |
| | aatttgtgatgctattgctttatttgtaaccattataagctgcaataaa |
| | caagttaacaacaacaattgcattcattttatgtttcaggttcagggg |
| | gaggtgtgggaggttttttaaagcaagtaaaacctctacaaatgtgg |
| | tatagttattaatagtaatcaattacggggtcattagttcatagccca |
| | tatatggagttccgcgttacataacttacggtaaatggcccgcctgg |
| | ctgaccgcccaacgacccccgcccattgacgtcaataatgacgtat |
| | gttcccatagtaacgccaatagggactttccattgacgtcaatgggt |
| | ggagtatttacggtaaactgcccacttggcagtacatcaagtgtatc |
| | atatgccaagtacgccccctattgacgtcaatgacggtaaatggccc |
| | gcctggcattatgcccagtacatgaccttatgggactttcctacttgg |
| | cagtacatctacgtattagtcatcgctattaccatggtgatgcggtttt |
| | ggcagtacatcaatgggcgtggatagcggtttgactcacggggattt |
| | ccaagtctccaccccattgacgtcaatgggagtttgttttggcaccaa |
| | aatcaacgggactttccaaaatgtcgtaacaactccgccccattgac |
| | gcaaatgggcggtaggcgtgtacggtgggaggtctatataagcaga |
| | gctggtttagtgaaccgtcagatcacccagctttcttgtacaaagtgg |
| | gccaccatggagacagatacactgctgctgtgggtgctgctcctctg |
| | ggtgccaggatctacaggcgacgtggtcatgacacagacccctctg |
| | acactgtccgtgaccatcggacagcctgcctccatctcctgcaagtc |
| | ctctcagtccctgctgcactctgacggcaagacctacctgaactggc |
| | tgctgcagaggcctggccagagtcctaagagactgatctacctggt |
| | gtccaagctggactctcggatccctgacagattcaccggctctggct |
| | ctggcaccgacttcaccctgaagatctccagagtggaagccgagga |
| | cctgggcgtgtactactgttggcagggcacccactttccacacacctt |
| | tggcgctggcacaaagctggaactgaagcggacagtggccgctcct |
| | tccgtgttcatcttcccaccttccgacgagcagctgaagtccggcac |
| | agcttctgtcgtgtgcctgctgaacaacttctaccctcgggaagcca |
| | aggtgcagtggaaggtggacaatgccctgcagtccggcaactccca |
| | agagtctgtgaccgagcaggactccaaggacagcacctacagcct |
| | gtcctccacactgaccctgtccaaggccgactacgagaagcacaag |
| | gtgtacgcctgcgaagtgacccatcagggcctgtctagccctgtgac |
| | caagtctttcaaccggggcgagtgttgacaactttattatacatagtt |
| | ggaattccgataatcaacctctggattacaaaatttgtgaaagattg |
| | actggtattcttaactatgttgctccttttacgctatgtggatacgctg |
| | ctttaatgcctttgtatcatgctattgcttcccgtatggctttcattttct |
| | cctccttgtataaatcctggttgctgtctctttatgaggagttgtggcc |
| | cgttgtcaggcaacgtggcgtggtgtgcactgtgtttgctgacgcaa |
| | cccccactggttggggcattgccaccacctgtcagctcctttccggga |
| | ctttcgctttccccctccctattgccacggcggaactcatcgccgcct |
| | gccttgcccgctgctggacaggggctcggctgttgggcactgacaat |
| | tccgtggtgttgtcggggaagctgacgtcctttccatggctgctcgcc |
| | tgtgttgccacctggattctgcgcgggacgtccttctgctacgtccctt |
| | cggccctcaatccagcggaccttccttcccgcggcctgctgccggct |
| | ctgcggcctcttccgcgtcttcgccttcgccctcagacgagtcggatc |
| | tccctttgggccgcctccccgcatcgggaattcctagagctcgctgat |
| | cagcctcgactgtgccttctagttgccagccatctgttgtttgcccctc |
| | ccccgtgccttccttgaccctggaaggtgccactcccactgtcctttc |
| | ctaataaaatgaggaaattgcatcgcattgtctgagtaggtgtcatt |
| | ctattctggggggtggggtggggcaggacagcaagggggaggatt |
| | gggaagagaatagcaggcatgctggggagggccgcaggaacccct |
| | agtgatggagttggccactccctctctgcgcgctcgctcgctcactga |
| | ggccgggcgaccaaaggtcgcccgacgcccgggctttgcccgggc |
| | ggcctcagtgagcgagcgagcgcgcagctgcctgcagg |
| | (SEQ ID NO: 10) |

### Example 2. Cell Culture

Human cerebral microvascular endothelial hCMEC/D3 cells were maintained in 75 cm² flasks precoated with 100 μg/mL rat tail collagen type-I (08-115, Merck) in EndoGRO-MV growth medium (Merck, SCME004) supplemented with all factors included in the kit, and 1 ng/mL bFGF (Merck, GF003), at 37° C. in a humidified atmosphere with 5% CO₂.

B.End3 and b.End5 mouse brain endothelioma cell lines were cultured in DMEM medium supplemented with Pen/strep and 10% FBS (growth medium). Cells were cultured in TC-treated 75 cm² flasks and detached with a trypsin-EDTA solution for passaging at a 1:10 ratio for subculture. Cells were incubated at 37° C. in a humidified atmosphere with 5% CO₂.

### Example 3. OrganoPlate® 3-Lane In Vitro BBB Model

The Organ® Plate® 3-lane system used for 3D in vitro BBB modeling encompasses 40 microfluidic cell culture structures embedded in a standard 384-well microtiter plate format. Each tissue chip is comprised of three lanes that are connected to corresponding wells of a microtiter plate that function as inlets and outlets to access the microfluidic culture. First, an extracellular matrix (ECM) gel of 4 mg/mL Collagen-I (rat tail, Merck) was prepared by mixing 1M HEPES, 37 g/L NaHCO₃, and 5 mg/mL Collagen-I, and introduced in the central lane. The phase guides were used to selectively pattern the ECM gel in the central lane by meniscus pinning. After ECM gelation (overnight or over weekend at 37° C., 5% CO₂), hCMEC/D3 cells were seeded in EndoGRO-MV growth medium supplemented with 1 ng/mL bFGF at a density of 40000 cells per chip in the top lane. Once the cells were attached, the plate was horizontally placed on an interval rocker that induced flow by reciprocal leveling between reservoirs, and incubated at 37° C., 5% CO₂ for at least 3 days to allow the formation of tubules. Medium changes were performed approximately every 3 days to maintain an optimal barrier integrity, which was controlled before every transduction or transcytosis experiment by permeability assays. Barrier function was assessed by perfusion with 0.5 mg/mL FITC-dextran (Sigma 46946, average 150 kDa; FD20S, average 20 kDa, and Sigma FD10S, average 10 kDa) in culture medium through the tube lumen, followed by the determination of fluorescence levels in the basal gel region, normalized to the fluorescence in the lumen. Fluorescence measurements were taken every 5 min during 1 h using an Incucyte live cell reader.

### Example 4. Transwell In Vitro BBB Model

For the Transwell-based in vitro BBB model, the apical side of a 24-well plate containing Corning Transwell membranes with a pore size of 0.4 μm (0.33 cm² culture area, Sigma) was first coated with rat tail collagen-I (Merck) for 1 h in a humidified incubator. hCMEC/D3 cells were then seeded in EndoGRO-MV growth medium supplemented with 1 ng/mL bFGF at a density of 100000 cells/cm2. Medium changes were performed approximately every 2-3 days, always maintaining an apical volume of 100 μL and 600 μL in basolateral. Permeability and Transendothelial electrical resistance (TEER) measurements were taken from day 4 after endothelial cell seeding, and cell monolayers were found to keep suitable barrier properties up to day 15 post seeding. For permeability measurements, the endothelial cell medium was replaced with 600 μL of fresh medium in the basolateral compartment. Then 100 μL of 0.25 mg/mL FITC (Sigma 46946, average 150 kDa; FD20S, average 20 kDa, and Sigma FD10S, average 10 kDa) in culture medium was added into the upper compartment and cells were incubated in a humidified incubator for 1 h. 100 μL fractions were then collected from the basolateral compartment and transferred to a Greiner black 96-well plate for fluorescence measurements using a Tecan Spark microplate reader. The apparent permeability was calculated according to the formula Papp=(ΔQ/Δt)×(1/AC0), where Papp is the apparent permeability coefficient (cm/min), ΔQ/Δt is the rate of permeation of dextran (μg/min) across the endothelial cell layer, A is the surface area of the cell layer (cm2) and CO is the initial dextran concentration (μg/ml) applied to the apical cell surface. For TEER measurements, the endothelial cell medium was replaced with 1050 μL fresh medium in the lower compartment and 325 μL in the upper compartment. An EVOM-3 epithelial voltohmmeter (WPI) was used to measure TEER.

### Example 5. AAV Transduction of hBMEC/hBMEC and Antibody Detection by Target Binding

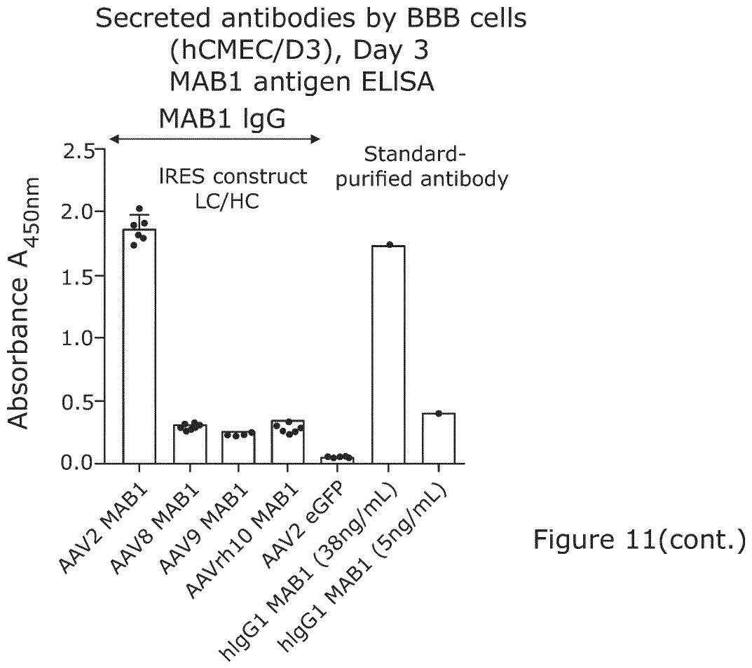

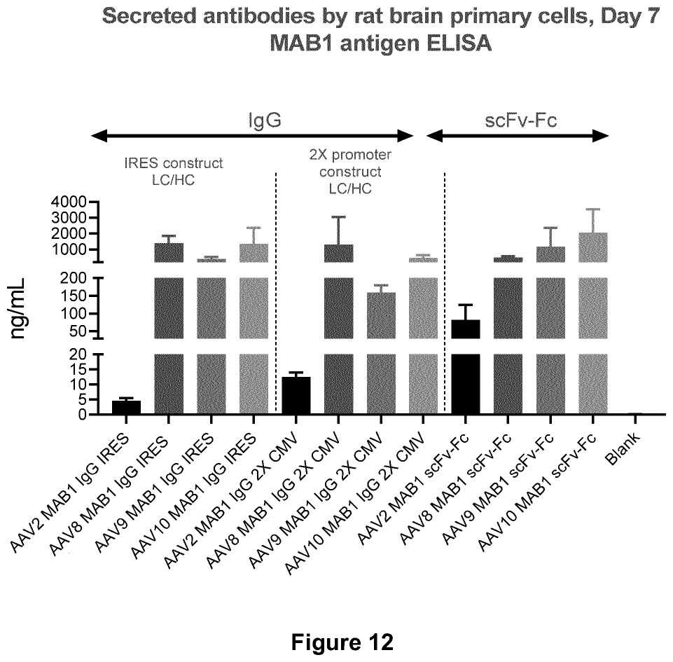

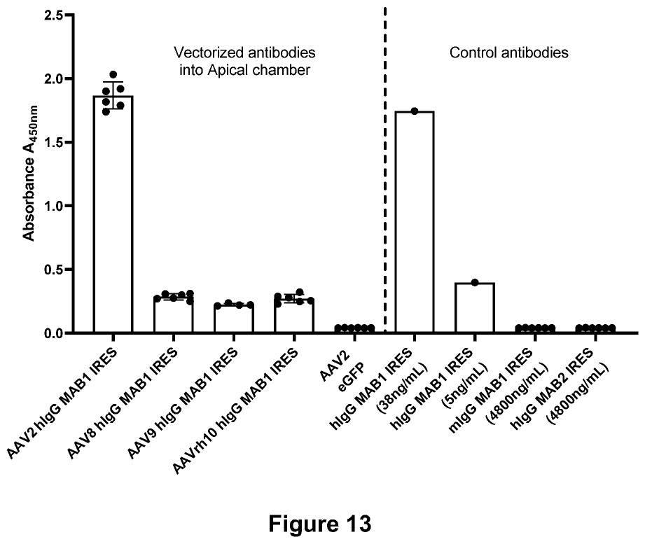

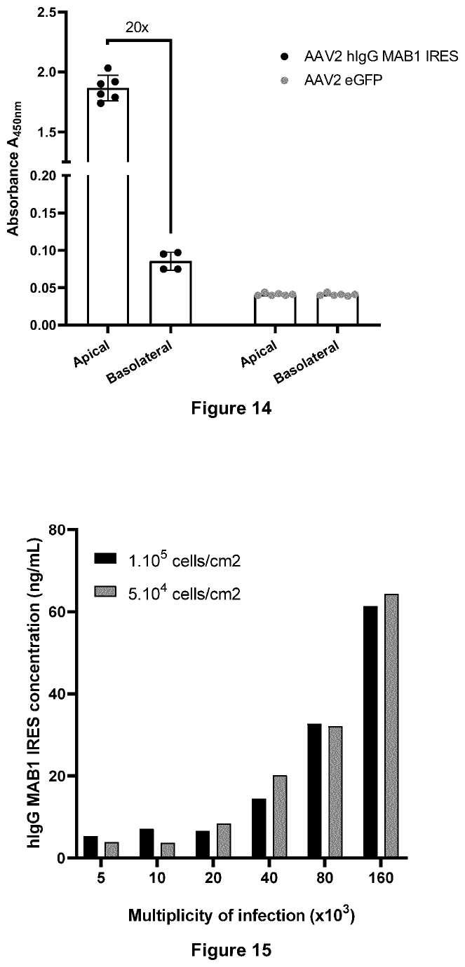

Antibody Secretion by hCMEC/D3 Cells Using Organ® Plate® BBB Model The established Organ® Plate® hCMEC/D3 in vitro BBB model was used to evaluate whether vectorized antibodies in AVV WT capsids could be efficiently secreted by hCMEC. The MAB1 (a human IgG1 isotype) antibody was vectorized into AAV2, AAV8; AAV9, and AAVrh10 capsids and transduced into hCMEC/D3 monolayers 24h after seeding into an Organ® Plate® 3-lane at a MOI of 50′000. Supernatants from both top lane (apical) and bottom lane (basolateral) were collected 3 days post-transduction and antibody concentrations in the apical and basolateral compartments were determined by binding to human full-length (FL) TDP-43 using an indirect ELISA. In brief, ELISA plate coating with 1 μg/ml human FL TDP-43 was performed overnight in carbonate buffer at 4° C. Plates were washed with 0.05% Tween-20/PBS and then blocked with 1% bovine serum albumin (BSA) in 0.05% Tween-20/PBS for 1 hour at 37° C. Collected antibody-containing supernatants were then added to the plate and incubated for 2 hours at 37° C. after which the plates were washed. An AP-conjugated anti-mouse IgG secondary antibody (Jackson, 115-055-206) was added diluted 1/1000 dilution in 0.05% Tween-20/PBS for 1 hour at 37° C. After the final wash, plates were incubated with pNPP solution and read at 405 nm after 1h using a plate reader (BioTek). MAB1 (a human IgG1 isotype) was detected in the apical supernatant of hCMEC/D3 monolayers transduced by all WT AAV capsids evaluated and retained binding to TDP-43 as illustrated in FIG. 13. MAB1 titers in the apical compartment were estimated around 40 ng/mL for AAV2, and around 4 ng/mL for AAV8; AAV9; and AAVrh10 serotypes. Relevant negative control (AAV2-eGFP) did not induce any signal in the TDP-43 ELISA assay. MAB1 (a human IgG1 isotype) transduced by AAV2 was also detected in the basolateral compartment at levels 20-fold lower compared to the apical side (FIG. 14), in accordance with the limited diffusion observed across the dense ECM layer in the Organ® Plate® (data not shown). The MAB1 (a human IgG1 isotype) AAV2 construct generated antibody titers in a dose-dependent fashion when transduced in hCMEC/D3 cells at MOIs between 5'000 to 160'000 as illustrated in FIG. 15. The Transwell in vitro BBB model was also used to further validate the delivery of vectorized antibodies. hCMEC/D3 monolayers were transduced 24h after seeding into 24-well Transwell inserts using the MAB1 AAV2 construct. Supernatants from both apical and basolateral sides were collected 3 days post-transduction and antibody concentrations in the apical and basolateral compartments were determined by binding to human FL TDP-43 using an indirect ELISA as described previously. This preliminary assessment suggested a relatively even secretion of the antibody toward both apical and basolateral sides, and therefore unpolarized secretion of the vectorized MAB1 antibody, as illustrated in FIG. 16.

### Example 6. Antibody Secretion by Mouse and Human Brain Microvasculature Endothelial Cell Lines

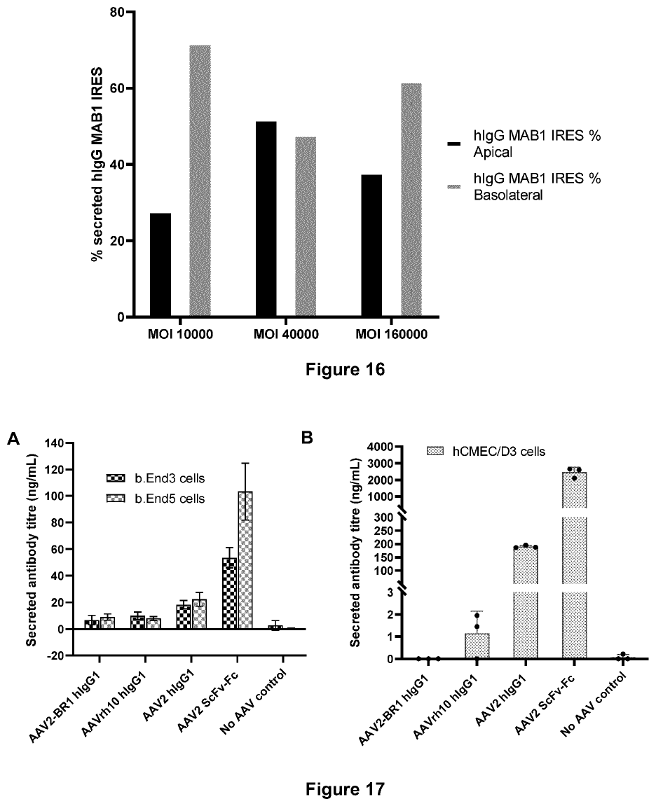

The immortalized hCMEC/D3, b.End3 and b.End5 cell lines were used to evaluate whether brain endothelial cells could produce high quality antibody. Different AAV vectors such as AAV2, AAV-BR1, and AAVrh10 were evaluated for their ability to deliver MAB1 antibody transgenes in to human and mouse cell lines. Expression of all antibodies (such as hIgG1 and scFv-Fc) was driven by a CMV promoter; an IRES element was used in between the genes encoding the LC and HC of the hIgG1 to achieve bicistronic antibody production. Cells were plated in 96-well culture plates at 100'000 cells/cm². Endothelial cell lines were then transduced 4h to 16h after plating at a MOI of 100'000 in growth medium. Cells were incubated overnight at 37° C., 5% CO₂ and medium was changed the next day to remove AAV particles. Cell culture supernatants were collected 7 days after post-transduction and secreted antibody titers were determined by Homogeneous Time Resolved Fluorescence (HTRF) using a hFc kit (PerkinElmer, Cisbio, 62HFCPEH) according to the manufacturer's instructions. This quantification method allowed detection of secreted and correctly folded antibodies. Purified recombinant MAB1 hIgG1 was used as a standard for antibody quantitation in culture medium. Fluorescence signals were read using a Tecan Spark® microplate reader (Em:317 nm; Ex:620 nm and 665 nm; 75 flashes; 400 μs integration time; 100 μs lag time). The interpolated secreted antibody titers are depicted in FIG. 17. MAB1 hIgG1 delivered by AAV2 was quantified at 20 ng/mL in (A) b.End3 and b.End5 mouse endothelioma cell line supernatants and at 200 ng/mL in (B) hCMEC/D3 cell supernatants. MAB1 scFv-Fc construct was produced and quantified at 50/100 ng/mL and 2500 ng/mL in supernatants, for b.End3/b.End5 and hCMEC/D3, respectively. Less than 10 ng/mL of MAB1 hIgG1 was obtained with the AAVrh10 and AAV-BR1 serotypes in all three cell lines. Obtained antibody levels show that brain endothelial cells produce correctly folded antibody, independently of the AAV capsid used or the format of the antibody. Expression titers were dependent on evaluated conditions and higher titers were observed for the human hCMEC/D3 cell line compared to both mouse cell lines (FIGS. 17A and B). Also, higher antibody titers were always observed for MAB1 scFv-Fc as compared to the whole MAB1 IgG1 counterpart when each were delivered by AAV2. In this experiment, AAV2 was more potent at delivering a transgene as compared to AAV-BR1.

### Example 7. Antibody Secretion by Human Primary Brain Microvasculature Endothelial Cells in a 3D Human BBB Model

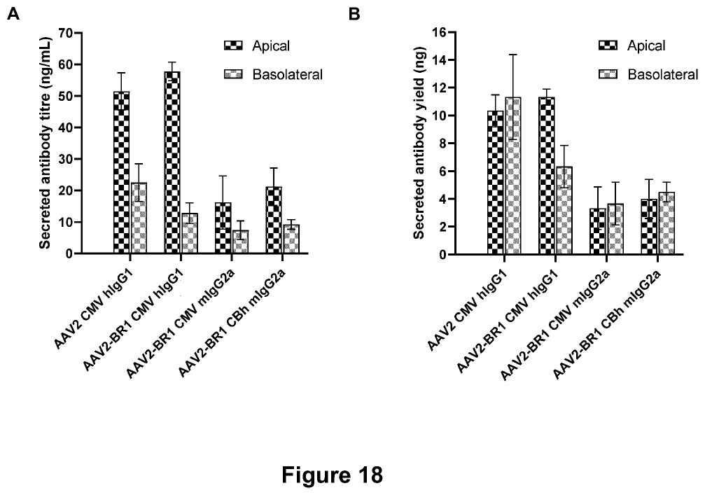

Antibody production by BBB cells was evaluated using a commercially available Transwell-based model composed of human primary brain endothelial microvasculature cells, astrocytes and pericytes, purchased from Neuromics (3D45002). The model was cultured following the manufacturer's instructions. In brief, the 24-well plate was thawed at day 0 and the freezing medium was replaced by warm growth medium (medium 1). After 3 hours incubation in a humidified incubator, medium 1 was removed and replaced by a second maintenance medium (medium 2). No further medium change was performed, and cells were kept in culture up to day 11 after thawing. AAV2 and AAV-BR1 vectors were evaluated to deliver a human (hIgG1) and mouse (mlgG2a) version of the MAB1 antibody transgene. Expression of the antibodies was driven by either CMV or CBh promoters; an IRES element was used between LC and HC to achieve bicistronic antibody production. Endothelial cells present in the cell culture inserts were transduced at a MOI of 100'000 in growth medium, as previously described with the AAV constructs, at day 4 after thawing. Supernatants from both apical and basolateral compartments were collected after 7 days post-transduction and secreted antibody titers were determined by HTRF using human Fc and mouse Fc kits (PerkinElmer Cisbio, 62HFCPEH and 6FMIGPEH) following the manufacturer's instructions. Purified recombinant MAB1 as hIgG1 or mlgG2a formats were respectively used as the standards for antibody quantitation and titrated in the same culture medium used for maintenance of the model. Fluorescence signals were read using a Tecan Spark® microplate reader (Em:317 nm; Ex:620 nm and 665 nm; 75 flashes; 400 μs integration time; 100 μs lag time). The interpolated secreted antibody titers are depicted in FIG. 18. Similar MAB1 hIgG1 titers were obtained for AAV2 and AAV-BR1 serotypes, with around 50 ng/mL in the apical compartment (cell culture insert) and 10-20 ng/mL on the basolateral side. AAV-BR1 delivery of the MAB1 mlgG2a isotype expressed under either CMV or CBh promoter displayed 2-to-3-fold lower titers as compared to the hIgG1 counterpart. A difference in tropism was observed with AAV-BR1, in the hCMEC/D3 cell line (FIG. 17A, no detectable MAB1 hIgG1 expression) when compared to the data from primary human brain microvasculature endothelial cells (FIGS. 18A and B). Whilst in vitro cell line data is predictive of in vivo effects, primary cell data is the preferred predictor of AAV tropism in vivo. FIG. 18B depicts the relative quantity of antibody determined in the apical (200 μL) and basolateral (500 μL) compartments for each AAV vector. The antibody repartition suggests a bilateral secretion of antibodies from the endothelial cell layer.

In summary, obtained antibody titers showed that primary brain endothelial cells produce correctly folded antibody, independently of the AAV capsid used and irrespective of the format of the antibody. As an important note, antibodies were detected in both the apical and basolateral sides, the latter observation mimicking brain parenchyma. Generated data validate the innovative method described herein and confirm that high quality IgG titers were achieved with this new delivery strategy.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes in connection with the invention.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

### REFERENCES

1. Villabona-Rueda, A., et al., The Evolving Concept of the Blood Brain Barrier (BBB): From a Single Static Barrier to a Heterogeneous and Dynamic Relay Center. Front Cell Neurosci, 2019. 13: p. 405.
2. Daneman, R. and A. Prat, The blood-brain barrier. Cold Spring Harb Perspect Biol, 2015. 7(1): p. a020412.
3. Pachter, J. S., H. E. de Vries, and Z. Fabry, The blood-brain barrier and its role in immune privilege in the central nervous system. J Neuropathol Exp Neurol, 2003. 62(6): p. 593-604.
4. Sweeney, M. D., et al., The role of brain vasculature in neurodegenerative disorders. Nat Neurosci, 2018. 21(10): p. 1318-1331.
5. St-Amour, I., et al., Brain bioavailability of human intravenous immunoglobulin and its transport through the murine blood-brain barrier. J Cereb Blood Flow Metab, 2013. 33(12): p. 1983-92.
6. Pardridge, W. M., Blood-Brain Barrier and Delivery of Protein and Gene Therapeutics to Brain. Front Aging Neurosci, 2019. 11: p. 373.
7. Thom, G., et al., Isolation of blood-brain barrier-crossing antibodies from a phage display library by competitive elution and their ability to penetrate the central nervous system. MAbs, 2018. 10(2): p. 304-314.
8. Zhang, W., et al., Differential expression of receptors mediating receptor-mediated transcytosis (RMT) in brain microvessels, brain parenchyma and peripheral tissues of the mouse and the human. Fluids Barriers CNS, 2020. 17(1): p. 47.
9. Gao, H., Progress and perspectives on targeting nanoparticles for brain drug delivery. Acta Pharm Sin B, 2016. 6(4): p. 268-86.
10. Teleanu, D. M., et al., Blood-Brain Delivery Methods Using Nanotechnology. Pharmaceutics, 2018. 10(4).
11. Chen, H., et al., Focused ultrasound-enhanced intranasal brain delivery of brain-derived neurotrophic factor. Sci Rep, 2016. 6: p. 28599.
12. Ahmad, E., et al., Evidence of nose-to-brain delivery of nanoemulsions: cargoes but not vehicles. Nanoscale, 2017. 9(3): p. 1174-1183.
13. Henchcliffe, C. and M. Parmar, Repairing the Brain: Cell Replacement Using Stem Cell-Based Technologies. J Parkinsons Dis, 2018. 8(s1): p. S131-S137.
14. Heller, L., et al., Secretion of proteins and antibody fragments from transiently transfected endothelial progenitor cells. Journal of cellular and molecular medicine, 2020. 24(15): p. 8772-8778.
15. Desai, N., Challenges in development of nanoparticle-based therapeutics. AAPS J, 2012. 14(2): p. 282-95.
16. Ka riolis, M. S., et al., Brain delivery of therapeutic proteins using an Fc fragment blood-brain barrier transport vehicle in mice and monkeys. Sci Transl Med, 2020. 12(545).
17. Ullman, J. C., et al., Brain delivery and activity of a lysosomal enzyme using a blood-brain barrier transport vehicle in mice. Sci Transl Med, 2020. 12(545).
18. Pothin, E., D. Lesuisse, and P. Lafaye, Brain Delivery of Single-Domain Antibodies: A Focus on VHH and VNAR. Pharmaceutics, 2020. 12(10).
19. Guo, X.-b., X. Deng, and Y. Wei, Homing of Cultured Endothelial Progenitor Cells and Their Effect on Traumatic Brain Injury in Rat Model. Scientific Reports, 2017. 7(1): p. 4164.
20. Guzman, R., M. Janowski, and P. Walczak, Intra-Arterial Delivery of Cell Therapies for Stroke. Stroke, 2018. 49(5): p. 1075-1082.
21. Tervo, D. G., et al., A Designer AAV Variant Permits Efficient Retrograde Access to Projection Neurons. Neuron, 2016. 92(2): p. 372-382.
22. Olanow, C. W., et al., Trophic factors for Parkinson's disease: To live or let die. Mov Disord, 2015. 30(13): p. 1715-24.
23. Tuszynski, M. H., et al., Nerve Growth Factor Gene Therapy: Activation of Neuronal Responses in Alzheimer Disease. JAMA Neurol, 2015. 72(10): p. 1139-47.
24. Merkel, S. F., et al., Trafficking of adeno-associated virus vectors across a model of the blood-brain barrier; a comparative study of transcytosis and transduction using primary human brain endothelial cells. J Neurochem, 2017. 140(2): p. 216-230.
25. Foust, K. D., et al., Intravascular AAV9 preferentially targets neonatal neurons and adult astrocytes. Nat Biotechnol, 2009. 27(1): p. 59-65.
26. Hanlon, K. S., et al., Selection of an Efficient AAV Vector for Robust CNS Transgene Expression. Mol Ther Methods Clin Dev, 2019. 15: p. 320-332.
27. Mathiesen, S. N., et al., CNS Transduction Benefits of AAV-PHP.eB over AAV9 Are Dependent on Administration Route and Mouse Strain. Mol Ther Methods Clin Dev, 2020. 19: p. 447-458.
28. Huang, Q., et al., Delivering genes across the blood-brain barrier: LY6A, a novel cellular receptor for AAV-PHP.B capsids. PLoS One, 2019. 14(11): p. e0225206.
29. Chan, K. Y., et al., Engineered AAVs for efficient noninvasive gene delivery to the central and peripheral nervous systems. Nat Neurosci, 2017. 20(8): p. 1172-1179.
30. Korbelin, J., et al., A brain microvasculature endothelial cell-specific viral vector with the potential to treat neurovascular and neurological diseases. EMBO Mol Med, 2016. 8(6): p. 609-25.
31. Ravindra Kumar, S., et al., Multiplexed Cre-dependent selection yields systemic AAVs for targeting distinct brain cell types. Nat Methods, 2020. 17(5): p. 541-550.
32. Elmer, B. M., et al., Gene delivery of a modified antibody to A8 reduces progression of murine Alzheimer's disease. PLoS One, 2019. 14(12): p. e0226245.
33. Sudol, K. L., et al., Generating differentially targeted amyloid-beta specific intrabodies as a passive vaccination strategy for Alzheimer's disease. Mol Ther, 2009. 17(12): p. 2031-40.
34. Ryan, D. A., et al., Abeta-directed single-chain antibody delivery via a serotype-1 AAV vector improves learning behavior and pathology in Alzheimer's disease mice. Mol Ther, 2010. 18(8): p. 1471-81.
35. Levites, Y., et al., Intracranial adeno-associated virus-mediated delivery of anti-pan amyloid beta, amyloid beta40, and amyloid beta42 single-chain variable fragments attenuates plaque pathology in amyloid precursor protein mice. J Neurosci, 2006. 26(46): p. 11923-8.
36. Kou, J., et al., Anti-Amyloid-8 Single-Chain Antibody Brain Delivery Via AAV Reduces Amyloid Load But May Increase Cerebral Hemorrhages in an Alzheimer's Disease Mouse Model. J Alzheimers Dis, 2011. 27(1): p. 23-38.
37. Liu, W., et al., Vectored Intracerebral Immunization with the Anti-Tau Monoclonal Antibody PHF1 Markedly Reduces Tau Pathology in Mutant Tau Transgenic Mice. J Neurosci, 2016. 36(49): p. 12425-12435.
38. Vitale, F., et al., Intramuscular injection of vectorized-scFvMC1 reduces pathological tau in two different tau transgenic models. Acta Neuropathol Commun, 2020. 8(1): p. 126.
39. Vitale, F., et al., Anti-tau conformational scFv MC1 antibody efficiently reduces pathological tau species in adult JNPL3 mice. Acta Neuropathol Commun, 2018. 6(1): p. 82.
40. Bell, R. D. and M. D. Ehlers, Breaching the blood-brain barrier for drug delivery. Neuron, 2014. 81(1): p. 1-3.
41. Sacramento, C. B., et al., Anti-Phospho-Tau Gene Therapy for Chronic Traumatic Encephalopathy. Hum Gene Ther, 2020. 31(1-2): p. 57-69.
42. Goodwin, M. S., et al., Utilizing minimally purified secreted rAAV for rapid and cost-effective manipulation of gene expression in the CNS. Mol Neurodegener, 2020. 15(1): p. 15.
43. Goodwin, M. S., et al., Anti-tau scFvs Targeted to the Cytoplasm or Secretory Pathway Variably Modify Pathology and Neurodegenerative Phenotypes. Mol Ther, 2020.
44. Pozzi, S., et al., Virus-mediated delivery of antibody targeting TAR DNA-binding protein-43 mitigates associated neuropathology. J Clin Invest, 2019. 129(4): p. 1581-1595.
45. Saunders, K. O., et al., Broadly Neutralizing Human Immunodeficiency Virus Type 1 Antibody Gene Transfer Protects Nonhuman Primates from Mucosal Simian-Human Immunodeficiency Virus Infection. J Virol, 2015. 89(16): p. 8334-45.
46. Ho, S. C., et al., IRES-mediated Tricistronic vectors for enhancing generation of high monoclonal antibody expressing CHO cell lines. J Biotechnol, 2012. 157(1): p. 130-9.
47. Liu, Z., et al., Systematic comparison of 2A peptides for cloning multi-genes in a polycistronic vector. Sci Rep, 2017. 7(1): p. 2193.
48. Yang, S., et al., Development of optimal bicistronic lentiviral vectors facilitates high-level TCR gene expression and robust tumor cell recognition. Gene Ther, 2008. 15(21): p. 1411-23.
49. Sharma, P., et al., 2A peptides provide distinct solutions to driving stop-carry on translational recoding. Nucleic Acids Res, 2012. 40(7): p. 3143-51.
50. Zhang, Y. and W. M. Pardridge, Mediated efflux of IgG molecules from brain to blood across the blood-brain barrier. J Neuroimmunol, 2001. 114(1-2): p. 168-72.
51. Boado, R. J., et al., Fusion antibody for Alzheimer's disease with bidirectional transport across the blood-brain barrier and abeta fibril disaggregation. Bioconjug Chem, 2007. 18(2): p. 447-55.
52. Deane, R., et al., IgG-assisted age-dependent clearance of Alzheimer's amyloid beta peptide by the blood-brain barrier neonatal Fc receptor. J Neurosci, 2005. 25(50): p. 11495-503.
53. Albright, B. H., et al., Mapping the Structural Determinants Required for AAVrh.10 Transport across the Blood-Brain Barrier. Mol Ther, 2018. 26(2): p. 510-523.
54. Albright, B. H., et al., Modulation of Sialic Acid Dependence Influences the Central Nervous System Transduction Profile of Adeno-associated Viruses. J Virol, 2019. 93(11).
55. Fukuchi, K., et al., Anti-Abeta single-chain antibody delivery via adeno-associated virus for treatment of Alzheimer's disease. Neurobiol Dis, 2006. 23(3): p. 502-11.
56. Elmer, B. M., et al., Gene delivery of a modified antibody to Abeta reduces progression of murine Alzheimer's disease. PLoS One, 2019. 14(12): p. e0226245.
57. Hay, C. E., et al., 2019.
58. Johnson, P. R., et al., Vector-mediated gene transfer engenders long-lived neutralizing activity and protection against SIV infection in monkeys. Nat Med, 2009. 15(8): p. 901-6.
59. Fuchs, S. P. and R. C. Desrosiers, Promise and problems associated with the use of recombinant AAV for the delivery of anti-HIV antibodies. Mol Ther Methods Clin Dev, 2016. 3: p. 16068.
60. Fuchs, S. P., et al., AAV-Delivered Antibody Mediates Significant Protective Effects against S/Vmac239 Challenge in the Absence of Neutralizing Activity. PLoS Pathog, 2015. 11(8): p. e1005090.
61. Martinez-Navio, J. M., et al., Long-Term Delivery of an Anti-SIV Monoclonal Antibody With AAV. Front Immunol, 2020. 11: p. 449.
62. Martinez-Navio, J. M., et al., Adeno-Associated Virus Delivery of Anti-HIV Monoclonal Antibodies Can Drive Long-Term Virologic Suppression. Immunity, 2019. 50(3): p. 567-575 e5.
63. Thomas, G., Furin at the cutting edge: from protein traffic to embryogenesis and disease. Nat Rev Mol Cell Biol, 2002. 3(10): p. 753-66.
64. Chng, J., et al., Cleavage efficient 2A peptides for high level monoclonal antibody expression in CHO cells. MAbs, 2015. 7(2): p. 403-12.
65. Kim, J. H., et al., High cleavage efficiency of a 2A peptide derived from porcine teschovirus-1 in human cell lines, zebrafish and mice. PLoS One, 2011. 6(4): p. e18556.
66. Velychko, S., et al., Fusion of Reprogramming Factors Alters the Trajectory of Somatic Lineage Conversion. Cell Rep, 2019. 27(1): p. 30-39 e4.
67. Fang, J., et al., Stable antibody expression at therapeutic levels using the 2A peptide. Nat Biotechnol, 2005. 23(5): p. 584-90.
68. Mizuguchi, H., et al., IRES-dependent second gene expression is significantly lower than cap-dependent first gene expression in a bicistronic vector. Mol Ther, 2000. 1(4): p. 376-82.
69. Fuchs, S. P., et al., Recombinant AAV Vectors for Enhanced Expression of Authentic IgG. PLoS One, 2016. 11(6): p. e0158009.

1. A vector comprising a polynucleotide encoding an antibody or antibody fragment for use in a method of treatment of a disease or disorder of the central nervous system (CNS) in a subject, wherein the vector transduces or transfects cells of the blood brain barrier (BBB) and the transduced or transfected BBB cells express the antibody or antibody fragment resulting in delivery of the antibody or antibody fragment into the CNS.

2. The vector for use according to claim 1, wherein the antibody or antibody fragment is delivered into the brain parenchyma, optionally wherein the antibody or antibody fragment is secreted into the brain parenchyma.

3. The vector for use according to claim 1 or claim 2, wherein the vector transduces or transfects endothelial cells of the BBB.

4. The vector for use according to any one of claims 1 to 3, wherein the vector transduces or transfects pericytes or astrocytes of the BBB.

5. The vector for use according to any one of the preceding claims, wherein the vector comprises a wild-type viral vector or an engineered viral vector.

6. The vector for use according to any one of the preceding claims, wherein the vector comprises a neurotropic vector.

7. The vector for use according to any one of the preceding claims, wherein the vector expresses a peptide, small molecule, antibody or antibody fragment thereof, protein, nanoparticle, lipid, oligonucleotide, aptamer or cationic molecule on the vector surface that targets the vector to the cells of the BBB.

8. The vector for use according to any one of the preceding claims, wherein the vector comprises modifications on the vector surface that targets the vector to the cells of the BBB.

9. The vector for use according to any one of the preceding claims, wherein the vector comprises organic nanomaterials such as, liposomes, exosomes, dendrimers, micelles, inorganic nanomaterials such as gold nanoparticles, silica nanoparticles or carbon nanotubes.

10. The vector for use according to any one of the preceding claims, wherein the vector is selected from: adeno associated virus (AAV), adenovirus, retrovirus, rhinovirus, lentivirus, herpes simplex virus (HSV) or any virus-like particle.

11. The vector for use according to any one of the preceding claims, wherein the vector is an AAV selected from: AAV serotype 1 (AAV1), AAV serotype 2 (AAV2), AAV serotype 8 (AAV8), AAV serotype 9 (AAV9) and AAV serotype 10 (AAV10).

12. The vector for use according to any one of the preceding claims, wherein the vector is an engineered AAV vector, wherein optionally
(i) the engineered AAV vector is an engineered AAV2 vector, preferably AAV-BR1; or
(ii) the engineered AAV vector is an engineered AAV9 vector, such as AAV-S, AAV-F, AAV-PHP.eB, AAV9-PHP-V1; or
(iii) the engineered AAV vector is an engineered AAV1 vector, such as AAV1RX, AAV1R6 or AAV1R7, or
(iv) the engineered AAV vector is an engineered AAV10 vector.

13. The vector for use according to any one of the preceding claims, wherein the vector is an AAV-BR1 or an AAV9-PHP-V1.

14. The vector for use according to any one of the preceding claims, wherein the disease or disorder of the CNS is selected from diseases associated with amyloid-beta protein, TDP-43-proteinopathies, alpha-synucleinopathies, Tauopathies, trinucleotide repeat disorders including poly-glutamine disorders such as Huntington's disease, brain-related cancers and tumors, epilepsy, psychiatric diseases, neuroinflammatory diseases, neuromuscular diseases, viral-induced encephalitis and diseases characterized by microglial dysfunction.

15. The vector for use according to any one of the preceding claims, wherein the disease or disorder of the CNS is selected from: Frontotemporal dementia (FTD), amyotrophic lateral sclerosis (ALS), Alzheimer's disease (AD), Parkinson's disease (PD), Chronic Traumatic Encephalopathy (CTE), limbic-predominant age-related TDP-43 encephalopathy (LATE), and multiple sclerosis.

16. The vector for use according to any one of the preceding claims, wherein the antibody or antibody fragment is selected from an: anti-ErbB2, anti-TDP-43 (NI-205), anti-Abeta (such as bapineuzumab, solanezumab, lecanemab, aducanumab, donanemab, gantenerumab or crenezumab), anti-ApoE4 (Apolipoprotein E4) and anti-DDX3X (ATP-dependent RNA helicase), anti-Tau (tilavonemab, gosuranemab, zagotenemab, semorinemab,
bepranemab, BIIB076, JNJ-63733657, Lu AF87908, PNT001, E-2814), anti-LINGO-1 (such as opicinumab), anti-alpha-aynuclein (cinpanemab, prasinezumab, MEDI-1341, Lu AF82422, BAN0805), anti-ASC (IC-100), anti-NLRP3, anti-C5 (ravulizumab, eculizumab), anti-Clq (ANX-005), anti-C3, anti-huntingtin (C-617, NI-302), anti-prion, anti-CD20 (such as ofatumumab, ocrelizumab, rituximab, BCD-132, ublituximab, BAT-4406F, AL-014), anti-PD-1 (IBC-Ab002) or anti-VEGF-A (bevacizumab, ranibizumab, brolucizumab, faricimab, vanucizumab) antibody or antibody fragment.

17. The vector for use according to any one of the preceding claims, wherein the vector is administered to the subject parenterally.

18. The vector for use according to claim 17, wherein the vector is administered to the subject by intravenous injection or intravenous infusion.

19. The vector for use according to any one of the preceding claims, wherein the polynucleotide comprises at least one promoter selected from a: cytomegalovirus (CMV) promoter, EF1A (Human Eukaryotic translation elongation factor 1 alpha 1), CAG (CMV early enhancer fused to modified chicken β-actin promoter), CBh (CMV early enhancer fused to modified chicken β-actin promoter), SV40 (Simian virus 40 enhancer/early promoter), GFAP (Human glial fibrillary acidic protein promoter), ATP1A2_1 (Na, K ATPase α2), CLDN_5 (Claudin 5), ADRB2_1 (Adrenoceptor beta 2), TNFRSF6B_1 (TNF receptor superfamily member 6b), PDYN_1 (prodynorphin), GH1_1 (Human growth hormone), OPALIN_1 (Opalin), SYN1_1 (Synapsin 1), CAMK2A_1 (Calcium/Calmodulin Dependent Protein Kinase II alpha), NEFH_1 (neurofilament heavy polypeptide), NEUROD6_1 (neuronal differentiation factor 6) or OLIG2_1 (oligodendrocyte transcription factor 2), a CMV early enhancer fused to either GFAP, ATP1A2_1, CLDN_5, ADRB2_1, TNFRSF6B_1, PDYN_1, GH1_1. OPALIN_1, SYN1_1, CAMK2A_1, NEFH_1, NEUROD6_1 or OLIG2_1 promoter, preferably a cytomegalovirus (CMV) promoter or CBh promoter, and wherein the at least one promoter is operably linked to a sequence encoding an antibody or antibody fragment.

20. The vector for use according to claim 19, wherein the at least one promoter is selected from a: ATP1A2_1 (Na, K ATPase α2), CLDN_5 (Claudin 5), ADRB2_1 (Adrenoceptor beta 2) and TNFRSF6B_1 (TNF receptor superfamily member 6b), optionally wherein the at least one promoter is operably linked to an enhancer such as a CMV early enhancer.

21. A method for delivery of an antibody or antibody fragment to the BBB in a subject, the method comprising administering a vector comprising a polynucleotide encoding the antibody or antibody fragment to the subject, wherein the method results in transduction or transfection of cells of the BBB and the transduced or transfected cells express the antibody or antibody fragment.

22. A method for delivery of an antibody or antibody fragment to the CNS in a subject, the method comprising administering a vector comprising a polynucleotide encoding the antibody or antibody fragment to the subject, wherein the method results in transduction or transfection of cells of the BBB and the transduced or transfected cells express the antibody or antibody fragment resulting in delivery of the antibody or antibody fragment into the CNS.

23. A method for treating a disease or disorder of the CNS in a subject, the method comprising administering a vector comprising a polynucleotide encoding an antibody or antibody fragment to the subject, wherein the method results in transduction or transfection of cells of the BBB and the transduced or transfected cells express the antibody or antibody fragment resulting in delivery of the antibody or antibody fragment into the CNS.

24. Use of a vector comprising a polynucleotide encoding an antibody or antibody fragment for the manufacture of a medicament for the treatment of a disease or disorder of the CNS in a subject, wherein the vector transduces or transfects cells of the blood brain barrier (BBB) and the transduced or transfected cells express the antibody or antibody fragment resulting in delivery of the antibody or antibody fragment into the CNS.

25. Use of a vector comprising a polynucleotide encoding an antibody or antibody fragment for delivery of the polynucleotide encoding the antibody or antibody fragment to the BBB of a subject wherein the vector transduces or transfects cells of the blood brain barrier (BBB) and the transduced or transfected cells express the antibody or antibody fragment resulting in delivery of the antibody or antibody fragment into the CNS.

26. The method or use according to any one of claims 21 to 25 further defined according to the features according to any one of claims 2 to 20.

27. A vector comprising an expression cassette comprising from 5′ to 3′: at least one promoter operably linked to a first gene encoding a light chain of an antibody or antibody fragment and to a second gene encoding a heavy chain of the antibody or antibody fragment for use in a method of treatment of a disease or disorder of the central nervous system (CNS) in a subject, wherein the vector transduces or transfects cells of the blood brain barrier (BBB) or the CNS and the transduced or transfected cells express the antibody or antibody fragment resulting in delivery of the antibody or antibody fragment into the CNS.

28. A vector comprising an expression cassette comprising from 5′ to 3′: a first promoter operably linked to a first gene encoding a light chain of an antibody or antibody fragment and a second promoter operably linked to a second gene encoding a heavy chain of the antibody or antibody fragment for use in a method of treatment of a disease or disorder of the central nervous system (CNS) in a subject, wherein the vector transduces or transfects cells of the blood brain barrier (BBB) or the CNS and the transduced or transfected cells express the antibody or antibody fragment resulting in delivery of the antibody or antibody fragment into the CNS.

29. The vector for use according to claim 27 or claim 28, wherein the antibody or antibody fragment is delivered into the brain parenchyma, optionally wherein the antibody or antibody fragment is secreted into the brain parenchyma.

30. The vector for use according to any one of claims 27 to 29, wherein the vector transduces or transfects cells of the CNS and the cells are selected from: brain endothelial cells, neurons, pericytes, astrocytes, oligodendrocytes, microglia and ependymal cells.

31. The vector for use according to any one of claims 27 to 30, wherein the vector transduces or transfects endothelial cells of the BBB.

32. The vector for use according to any one of claims 27 to 31, wherein the vector transduces or transfects pericytes or astrocytes of the BBB.

33. The vector for use according to any one of claims 27 to 32, wherein the antibody or antibody fragment is secreted into the CNS, preferably secreted into the brain parenchyma.

34. The vector for use according to any one of claims 27 to 33, wherein the vector comprises modifications on the vector surface that targets the vector to the cells of the BBB.

35. The vector for use according to any one of claims 27 to 34, wherein the vector expresses a peptide, small molecule, antibody or antibody fragment thereof, protein, nanoparticle, lipid, oligonucleotide, aptamer or cationic molecule on the vector surface that targets the vector to the cells of the BBB or the CNS.

36. The vector for use according to any one of claims 27 to 35, wherein the vector comprises a neurotropic vector.

37. The vector for use according to any one of claims 27 to 36, wherein the vector comprises organic nanomaterials such as, liposomes, exosomes, dendrimers, and micelles or inorganic nanomaterials such as gold nanoparticles, silica nanoparticles and carbon nanotubes.

38. The vector for use according to any one of claims 27 to 37, wherein the vector comprises a wild-type viral vector or an engineered viral vector.

39. The vector for use according to any one of claims 27 to 38, wherein the vector is selected from: adeno associated virus (AAV), adenovirus, retrovirus, rhinovirus, lentivirus, herpes simplex virus (HSV) or a virus-like particle.

40. The vector for use according to any one of claims 27 to 39, wherein the vector is an AAV selected from: AAV serotype 1 (AAV1), AAV serotype 2 (AAV2), AAV serotype 8 (AAV8), AAV serotype 9 (AAV9) and AAV serotype 10 (AAV10).

41. The vector for use according to any one of claims 27 to 40, wherein the vector is an engineered AAV vector, wherein optionally
(i) the engineered AAV vector is an engineered AAV2 vector, preferably AAV-BR1; or
(ii) the engineered AAV vector is an engineered AAV9 vector, such as AAV-S, AAV-F, AAV-PHP.eB, AAV9-PHP-V1; or
(iii) the engineered AAV vector is an engineered AAV1 vector, such as AAV1RX, AAV1R6 or AAV1R7, or
(iv) the engineered AAV vector is an engineered AAV10 vector.

42. The vector for use according to any one of claims 27 to 41, wherein the disease or disorder of the CNS is selected from diseases associated with amyloid-beta protein, TDP-43-proteinopathies, alpha-synucleinopathies, Tauopathies, trinucleotide repeat disorders including poly-glutamine disorders such as Huntington's disease, brain-related cancers and tumors, epilepsy, psychiatric diseases, neuroinflammatory diseases, neuromuscular diseases, viral-induced encephalitis and diseases characterized by microglial dysfunction.

43. The vector for use according to any one of claims 27 to 42, wherein the disease or disorder of the CNS is selected from: Frontotemporal dementia (FTD), amyotrophic lateral sclerosis (ALS), Alzheimer's disease (AD), Parkinson's disease (PD), Chronic Traumatic Encephalopathy (CTE), limbic-predominant age-related TDP-43 encephalopathy (LATE) and multiple sclerosis.

44. The vector for use according to any one of claims 27 to 43, wherein the antibody or antibody fragment is selected from an: anti-ErbB2, anti-TDP-43 (NI-205), anti-Abeta (such as bapineuzumab, solanezumab, lecanemab, aducanumab, donanemab, gantenerumab or crenezumab), anti-ApoE4 (Apolipoprotein E4) and anti-DDX3X (ATP-dependent RNA helicase), anti-Tau (tilavonemab, gosuranemab, zagotenemab, semorinemab, bepranemab, BIIB076, JNJ-63733657, Lu AF87908, PNT001, E-2814), anti-LINGO-1 (such as opicinumab), anti-alpha-synuclein (cinpanemab, prasinezumab, MEDI-1341, Lu AF82422, BAN0805), anti-ASC (IC-100), anti-NLRP3, anti-C5 (ravulizumab, eculizumab), anti-Clq (ANX-005), anti-C3, anti-huntingtin (C-617, NI-302), anti-prion, anti-CD20 (such as ofatumumab, ocrelizumab, rituximab, BCD-132, ublituximab, BAT-4406F, AL-014), anti-PD-1 (IBC-Ab002) or anti-VEGF-A (bevacizumab, ranibizumab, brolucizumab, faricimab, vanucizumab).

45. The vector for use according to any one of claims 27 to 44, wherein the vector is administered to the subject parenterally.

46. The vector for the use according to claim 45, wherein the vector is administered to the subject by intravenous injection or intravenous infusion.

47. The vector for the use according to claims 27 to 46, wherein the expression cassette further comprises either an internal ribosomal entry site (IRES) or furin-2A cleavage site after the first gene encoding the light chain of the antibody or antibody fragment and before the second gene encoding the heavy chain of the antibody or antibody fragment.

48. The vector for use according to claim 47, wherein the IRES is derived from encephalomyocarditis virus, and optionally comprises SEQ ID NO: 1 or SEQ ID NO: 8.

49. The vector for use according to any one of claims 27 to 48, wherein the first gene encoding the light chain of the antibody or antibody fragment further comprises a secretion peptide and/or the second gene encoding the heavy chain of the antibody or antibody fragment further comprises a secretion peptide.

50. The vector for use according to any one of claims 27 to 49, wherein the at least one promoter and/or the first promoter and/or the second promoter is selected from a: cytomegalovirus (CMV) promoter, EF1A (Human Eukaryotic translation elongation factor 1 alpha 1), CAG (CMV early enhancer fused to modified chicken β-actin promoter), CBh (CMV early enhancer fused to modified chicken β-actin promoter), SV40 (Simian virus 40 enhancer/early promoter), GFAP (Human glial fibrillary acidic protein promoter), ATP1A2_1 (Na, K ATPase α2), CLDN_5 (Claudin 5), ADRB2_1 (Adrenoceptor beta 2), TNFRSF6B_1 (TNF receptor superfamily member 6b), PDYN_1 (prodynorphin), GH1_1 (Human growth hormone), OPALIN_1 (Opalin), SYN1_1 (Synapsin 1), CAMK2A_1 (Calcium/Calmodulin Dependent Protein Kinase II alpha), NEFH_1 (neurofilament heavy polypeptide), NEUROD6_1 (neuronal differentiation factor 6) or OLIG2_1 (oligodendrocyte transcription factor 2), a CBh, a CMV early enhancer fused to either GFAP, ATP1A2_1, CLDN_5, ADRB2_1, TNFRSF6B_1, PDYN_1, GH1_1. OPALIN_1, SYN1_1, CAMK2A_1, NEFH_1, NEUROD6_1 or OLIG2_1 promoter, preferably a cytomegalovirus (CMV) promoter or CBh promoter, and wherein the at least one promoter is operably linked to a sequence encoding an antibody or antibody fragment.

51. The vector for use according to claim 50, wherein the at least one promoter and/or the first promoter and/or the second promoter is selected from a: ATP1A2_1 (Na, K ATPase α2), CLDN_5 (Claudin 5), ADRB2_1 (Adrenoceptor beta 2) and TNFRSF6B_1 (TNF receptor superfamily member 6b), optionally wherein the at least one promoter and/or the first promoter and/or the second promoter is operably linked to an enhancer such as a CMV early enhancer.

52. The vector for use according to any one of claims 27 to 51, wherein the expression cassette comprises a sequence selected from: SEQ ID NO: 1, SEQ ID NO: 2, SEQ ID NO: 3, SEQ ID NO: 4, SEQ ID NO: 5, SEQ ID NO: 6 SEQ ID NO: 7, SEQ ID NO: 8, SEQ ID NO: 9 or SEQ ID NO: 10 or a sequence having at least 80%, 85%, 90%, 95%, 97%, 98% or 99% identity therewith.

53. A method of reducing antibody or antibody fragment aggregation, improving antibody or antibody fragment maturation and/or quality, wherein the method comprises:
(i) transforming cells with an expression cassette comprising from 5′ to 3′: at least one promoter operably linked to a first gene encoding a light chain of an antibody or antibody fragment and to a second gene encoding a heavy chain of the antibody or antibody fragment and wherein the expression cassette further comprises an internal ribosomal entry site (IRES) after the first gene encoding the light chain of the antibody or antibody fragment and before the second gene encoding the heavy chain of the antibody or antibody fragment; or with an expression cassette comprising from 5′ to 3′: a first promoter operably linked to a first gene encoding a light chain of an antibody or antibody fragment and a second promoter operably linked to a second gene encoding a heavy chain of the antibody or antibody fragment; and
(ii) maintaining the transformed cells under conditions suitable for antibody or antibody fragment production.

54. A method of increasing antibody or antibody fragment titer, wherein the method comprises:
(i) transforming cells with an expression cassette comprising from 5′ to 3′: at least one promoter operably linked to a first gene encoding a light chain of an antibody or antibody fragment and to a second gene encoding a heavy chain of the antibody or antibody fragment and wherein the expression cassette further comprises either an internal ribosomal entry site (IRES) or self (e.g. furin-2A) cleavage site after the first gene encoding the light chain of the antibody or antibody fragment and before the second gene encoding the heavy chain of the antibody or antibody fragment; or with an expression cassette comprising from 5′ to 3′: a first promoter operably linked to a first gene encoding a light chain of an antibody or antibody fragment and a second promoter operably linked to a second gene encoding a heavy chain of the antibody or antibody fragment; and
(ii) maintaining the transformed cells under conditions suitable for antibody or antibody fragment production.

55. A method of reducing unwanted antibody or antibody fragment immunogenicity and/or adverse effects associated with antibody or antibody fragment therapy, wherein the method comprises:
(i) transforming cells with an expression cassette comprising from 5′ to 3′: at least one promoter operably linked to a first gene encoding a light chain of an antibody or antibody fragment and to a second gene encoding a heavy chain of the antibody or antibody fragment and wherein the expression cassette further comprises an internal ribosomal entry site (IRES) after the first gene encoding the light chain of the antibody or antibody fragment and before the second gene encoding the heavy chain of the antibody or antibody fragment or with an expression cassette comprising from 5′ to 3′: a first promoter operably linked to a first gene encoding a light chain of an antibody or antibody fragment and a second promoter operably linked to a second gene encoding a heavy chain of the antibody or antibody fragment; and
(ii) maintaining the transformed cells under conditions suitable for antibody or antibody fragment production.

56. The method according to any one of claims 53 to 55, wherein the antibody or antibody fragment are free of self-cleavage elements.

57. The method according to any one of claims 53 to 56 wherein the expression cassette is comprised in a vector and the method is as further defined according to the features according to any one of claims 29 to 52.

58. An antibody or antibody fragment obtained by the methods according to any one of claims 53 to 57.

59. A viral vector comprising an engineered AAV2 vector, preferably AAV-BR1 or an engineered AAV9 vector, such as AAV-S, AAV-F, AAV-PHP.eB or AAV9-PHP-V1 or an engineered AAV1 vector, such as AAV1RX, AAV1R6, AAV1R7 or an engineered AAV10 vector and wherein the engineered viral vector comprises an expression cassette comprising from 5′ to 3′: at least one promoter operably linked to a first gene encoding a light chain of an antibody or antibody fragment and to a second gene encoding a heavy chain of the antibody or antibody fragment and further comprises an IRES after the first gene encoding the light chain of the antibody or antibody fragment and before the second gene encoding the heavy chain of the antibody or antibody fragment.

60. A viral vector comprising an engineered AAV2 vector, preferably AAV-BR1 or an engineered AAV9 vector, such as AAV-S, AAV-F, AAV-PHP.eB or AAV9-PHP-V1 or an engineered AAV1 vector, such as AAV1RX, AAV1R6 or AAV1R7 or an engineered AAV10 vector and wherein the engineered viral vector comprises an expression cassette comprising: from 5′ to 3′: a first promoter operably linked to a first gene encoding a light chain of an antibody or antibody fragment and a second promoter operably linked to a second gene encoding a heavy chain of the antibody or antibody fragment.

61. The engineered viral vector according to claim 59 or 60, wherein the engineered viral vector is AAV-BR1 or AAV9-PHP-V1.

62. The viral vector according to any one of claims 59 to 61, as further defined according to any one of claims 29 to 52.
